(19) United States
(12) Patent Application Publication
(10) Pub. No.: US 2010/0120669 A1
(43) Pub. Date: May 13, 2010

(54) THIADIAZOLE DERIVATIVES, INHIBITORS OF STEAROYL-COA DESATURASE

(76) Inventors: Anne Marie Jeanne Bouillot, Les Ulis (FR); Thierry Boyer, Les Ulis (FR); Alain Claude-Marie Daugan, Les Ulis (FR); Anthony William Dean, Hertfordshire (GB); Martin Christian Fillmore, Hertfordshire (GB); Yann Lamotte, Les Ulis (FR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/528,647

(22) PCT Filed: Feb. 26, 2008
(86) PCT No.: PCT/EP2008/052276

§ 371 Date: Aug. 26, 2009

(30) Foreign Application Priority Data
Feb. 28, 2007 (GB) 0703906.8
Aug. 10, 2007 (GB) 0715654.0

(51) Int. Cl. A61K 38/12; C07D 217/00; A61K 31/47; A61K 31/56; A61K 31/593; A61K 31/65; A61P 9/00; A61P 3/04; A61P 3/10; A61P 35/00; A61P 25/00

(52) U.S. Cl. 514/10; 546/146; 514/307; 514/171; 514/167; 514/154

(57) ABSTRACT

The present invention relates to substituted thiadiazole compounds of the formula (I):

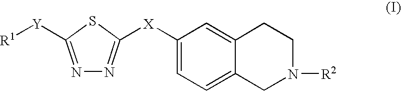

and pharmaceutically acceptable salts thereof, to pharmaceutical compositions containing them and their use in medicine. In particular, the invention relates to compounds for modulating SCD activity.

### FIELD OF THE INVENTION

The present invention relates to a novel class of compounds believed to be inhibitors of stearoyl-CoA desaturase (SCD), compositions comprising said compounds, methods of synthesis and uses for such compounds in treating and/or preventing various diseases, including those mediated by SCD enzyme, such as diseases related to elevated lipid levels, cardiovascular disease, diabetes, obesity, metabolic syndrome, skin disorders such as acne, diseases or conditions related to cancer and the treatment of symptoms linked to the production of the amyloid plaque-forming Aβ42 peptide such as Alzheimer's disease and the like.

### BACKGROUND OF THE INVENTION

Acyl desaturase enzymes catalyze the formation of double bonds in fatty acids derived from either dietary sources or de novo synthesis in the liver. Mammals synthesise at least three fatty acid desaturases of differing chain length that specifically catalyze the addition of double bonds at the delta-9, delta-6, and delta-5 positions. Stearoyl-CoA desaturases (SCDs) introduce a double bond in the C9-C10 position of saturated fatty acids. The preferred substrates for the enzymes are palmitoyl-CoA (16:0) and stearoyl-CoA (18:0), which are converted to palmitoleoyl-CoA (16:1) and oleoyl-CoA (18:1), respectively. The resulting mono-unsaturated fatty acids may then be employed in the preparation of phospholipids, triglycerides, and cholesteryl esters, in vivo.

A number of mammalian SCD genes have been cloned. For example, two genes have been cloned from rats (SCD1, SCD2) and four SCD genes have been isolated from mice (SCD1, 2, 3 and 4). While the basic biochemical roles of SCD has been known in rats and mice since the 1970's (Jeffcoat, R et al., Elsevier Science (1984), Vol 4, pp. 85-112; de Antueno, R J, Lipids (1993), Vol. 28, No. 4, pp. 285-290), it has only recently been directly implicated in human diseases processes.

A single SCD gene, SCD1, has been characterized in humans. SCD1 is described in Brownlie et al, WO 01/62954. A second human SCD isoform has been identified, and because it bears little sequence homology to known mouse or rat isoforms it has been named human SCD5 or hSCD5 (WO 02/26944).

Whilst not wishing to be bound by theory, it is thought that inhibition of the activity of SCD in vivo can be used to ameliorate and/or treat one or more diseases such as dyslipidemia, hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia, metabolic syndrome; other cardiovascular diseases e.g. peripheral vascular disease, reperfusion injury, angioplastic restenosis, hypertension, vascular complications of diabetes, thrombosis; hepatic steatosis, non-alcoholic steatohepatitis (NASH) and other diseases related to accumulation of lipids in the liver.

An SCD-mediated disease or condition also includes a disorder of polyunsaturated fatty acid (PUFA) disorder, or a skin disorder, including but not limited to eczema, acne, psoriasis, keloid scar formation or prevention, diseases related to production or secretions from mucous membranes, such as monounsaturated fatty acids, wax esters, and the like (US2006/0205713A1, WO2007/046868, WO2007/046867). SCD has been shown to play a physiological role in cholesterol homeostasis and the de novo biosynthesis of cholesterol esters, triglycerides and wax esters required for normal skin and eyelid function and therefore may be useful in the treatment of acne and other skin conditions (Makoto et al. J of Nutrition (2001), 131(9), 2260-2268, Harrison et al. J of Investigative Dermatology (2007) 127(6), 1309-1317).

An SCD-mediated disease or condition also includes but is not limited to a disease or condition which is, or is related to cancer, neoplasia, malignancy, metastases, tumours (benign or malignant), carcinogenesis, hepatomas and the like (US2006/0205713A1, WO2007/046868, WO2007/046867). Recently, SCD-1 has been identified as playing a role in human tumor cell survival and therefore has potential as an anticancer target (Morgan-Lappe et al. 2007 Cancer Res. 67(9) 4390-4398).

It has been shown that overexpression of Steroyl-CoA desaturase (SCD) in human cells in culture leads to a specific increase in the production of the amyloid plaque-forming Aβ42 peptide, and conversely, that reductions in SCD activity in human cells in culture leads to a specific decrease in the production of Aβ42. Therefore, SCD inhibitors may also be useful for treating, delaying the onset of symptoms, or slowing the progression of symptoms of mild cognitive impairment (MCI), Alzheimer's Disease (AD), cerebral amyloid angiopathy (CAA) or dementia associated with Down Syndrome (DS) and other neurodegenerative diseases characterized by the formation or accumulation of amyloid plaques comprising Aβ42 (US2007/0087363A1; Myriad Genetics).

WO2005/011657 describes certain piperazine derivatives useful for modulating SCD activity.

The present invention provides a compound of formula (I) for inhibiting SCD activity:

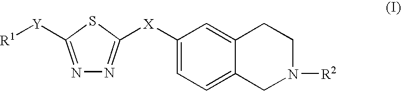

wherein:
X represents —CONH—, —NHCO— or —N(CH₃)CO—,
R¹ represents:
(i) a substituent selected from: H, —C₁₋₆alkyl or —C₃₋₆cycloalkyl,
(ii) —C₆₋₁₀aryl (such as phenyl or naphthyl) optionally substituted by one, two or three groups independently selected from:
   (a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro),
   (b) —C₆₋₁₀aryl (such as phenyl), —C₅₋₁₀heteroaryl or —C₅₋₁₀heterocyclyl, wherein the —C₆₋₁₀aryl, —C₅₋₁₀heteroaryl or —C₅₋₁₀heterocyclyl ring is optionally substituted by one, two or three groups independently selected from: —C₁₋₆alkyl (such as —CH₃), —C₁₋₆haloalkyl (such as —CF₃), —C₁₋₆alkoxy (such as —OCH₃), —OR³, —CN or halogen (such as chloro, bromo or fluoro),
(iii) —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
   (a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro),
   (b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl (such as —CH₃), —C₁₋₆haloalkyl (such as —CF₃), —C₁₋₆alkoxy (such as —OCH₃), —OR³, —CN or halogen (such as chloro, bromo or fluoro),
Y represents —(CH₂)ₘ—, —O(CH₂)ₘ— —NR⁷(CH₂)ₘ—,
R² represents H, —C₁₋₆alkyl, —C(═O)C₁₋₆alkyl, —C(═O)C₃₋₆cycloalkyl, —C(═O)C₆₋₁₀aryl, —C(═O)C₁₋₆alkylOH, —COC₁₋₃alkylNR⁴R⁵ or —C₅heteroarylR⁶,
R³ represents —C₁₋₆haloalkyl (such as —CF₃) or —C₃₋₆cycloalkyl,
R⁴ represents H or —C₁₋₃alkyl (such as —CH₃),
R⁵ represents H or —C₁₋₃alkyl (such as —CH₃),
R⁶ represents —C₁₋₃alkylOH,
R⁷ represents H or —C₁₋₃alkyl (such as —CH₃), and
m represents 1-4
   or a pharmaceutically acceptable salt thereof.

The said compounds have been found to inhibit SCD activity and may therefore be useful in the treatment of SCD-mediated diseases such as diseases or conditions caused by or associated with an abnormal plasma lipid profile including dyslipidemia, hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia and metabolic syndrome; other cardiovascular diseases e.g. peripheral vascular disease, reperfusion injury, angioplastic restenosis, hypertension, vascular complications of diabetes, thrombosis, hepatic steatosis, non-alcoholic steatoheptatis (NASH) and other diseases related to accumulation of lipids in the liver; skin disorders e.g. eczema, acne, psoriasis, keloid scar formation or prevention, and diseases related to production or secretions from mucous membranes; cancer, neoplasia, malignancy, metastases, tumours (benign or malignant), carcinogenesis, hepatomas and the like; mild cognitive impairment (MCI), Alzheimer's Disease (AD), cerebral amyloid angiopathy (CAA) or dementia associated with Down Syndrome (DS) and other neurodegenerative diseases characterized by the formation or accumulation of amyloid plaques comprising Aβ42.

In one aspect of the invention,
X represents —CONH— or —NHCO—;
R¹ represents:
(i) a substituent selected from: H, —C₁₋₆alkyl or —C₃₋₆cycloalkyl,
(ii) —C₆₋₁₀aryl (such as phenyl) optionally substituted by one, two or three groups independently selected from:
   (a) —C₁₋₆alkyl, —C₁₋₆haloalkyl (such as —CF₃), —C₃₋₆cycloalkyl, —C₁₋₆alkoxy, —OC₁₋₆haloalkyl (such as —OCF₃), —OR³, —CN or halogen (such as chloro, bromo or fluoro),
   (b) —C₆₋₁₀aryl (such as phenyl), —C₅₋₁₀heteroaryl or —C₅₋₁₀heterocyclyl, wherein the —C₆₋₁₀aryl, —C₅₋₁₀heteroaryl or —C₅₋₁₀heterocyclyl ring is optionally substituted by one, two or three groups independently selected from: —C₁₋₆alkyl, —OR³, —C₁₋₆haloalkyl (such as —CF₃), —CN or halogen (such as chloro, bromo or fluoro),
(iii) —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
   (a) —C₁₋₆alkyl, —C₁₋₆haloalkyl (such as —CF₃), —C₃₋₆cycloalkyl, —C₁₋₆alkoxy, —OC₁₋₆haloalkyl (such as —OCF₃), —OR³, —CN or halogen (such as chloro, bromo or fluoro),
   (b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl, —OR³, —C₁₋₆alkoxy —C₁₋₆haloalkyl (such as —CF₃), —CN or halogen (such as chloro, bromo or fluoro),
Y represents —(CH₂)ₘ—, —O(CH₂)ₘ— or —NR⁴(CH₂)ₘ—,
R² represents H, —C₁₋₆alkyl, —C(═O)C₁₋₆alkyl, —C(═O)C₃₋₆cycloalkyl or —C(═O)C₆₋₁₀aryl,
R³ represents —C₁₋₆haloalkyl (such as —CF₃) or —C₃₋₆cycloalkyl,
R⁴ represents H or —CH₃, and
m represents 1-4
   or a pharmaceutically acceptable salt thereof.

In one aspect of the invention, X represents —NHCO—. In another aspect of the invention, X represents —CONH—. In another aspect of the invention, X represents —N(CH₃)CO—.

In one aspect of the invention, R¹ represents:
(i) a substituent selected from: H or —C₃₋₆cycloalkyl,
(ii) —C₆₋₁₀aryl (such as phenyl or naphthyl) optionally substituted by one, two or three groups independently selected from:
   (a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro),
   (b) —C₆₋₁₀aryl (such as phenyl), —C₆₋₁₀heteroaryl or —C₆₋₁₀heterocyclyl, wherein the —C₆₋₁₀aryl, —C₆₋₁₀heteroaryl or —C₆₋₁₀heterocyclyl ring is optionally substituted by one, two or three groups independently selected from: —C₁₋₆alkyl (such as —CH₃), —C₁₋₆haloalkyl (such as —CF₃), —C₁₋₆alkoxy (such as —OCH₃), —OR³, —CN or halogen (such as chloro, bromo or fluoro),
(iii) —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
   (a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro),
   (b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl (such as —CH₃), —C₁₋₆haloalkyl (such as —CF₃), —C₁₋₆alkoxy (such as —OCH₃), —OR³, —CN or halogen (such as chloro, bromo or fluoro).

In another aspect of the invention, R¹ represents —C₃₋₆cycloalkyl. In another aspect of the invention, R¹ represents cyclohexane.

In another aspect of the invention, R¹ represents —C₆₋₁₀aryl optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro), or
(b) —C₆₋₁₀aryl (such as phenyl) optionally substituted by one, two or three groups independently selected from: —C₁₋₆alkyl (such as —CH₃), —C₁₋₆alkoxy (such as —OCH₃), —OR³, —C₁₋₆haloalkyl (such as —CF₃), —CN or halogen (such as chloro, bromo or fluoro).

In another aspect of the invention, R¹ represents —C₆₋₁₀aryl optionally substituted by: one, two or three groups independently selected from:
(a) —C₁₋₆alkyl, —C₁₋₆haloalkyl (such as —CF₃), —C₃₋₆cycloalkyl, —C₁₋₆alkoxy, —OR³, —CN, halogen or
(b) —C₆₋₁₀aryl (such as phenyl) optionally substituted by one, two or three groups independently selected from: —C₁₋₆alkyl, —OR³, —C₁₋₆alkoxy, —C₁₋₆haloalkyl (such as —CF₃), —CN or halogen.

In another aspect of the invention, R¹ represents phenyl optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro),
(b) or phenyl optionally substituted by one, two or three groups independently selected from: —C₁₋₆alkyl (such as —CH₃), —C₁₋₆alkoxy (such as —OCH₃), —OR³, —C₁₋₆haloalkyl (such as —CF₃), —CN or halogen (such as chloro, bromo or fluoro).

In another aspect of the invention, R¹ represents phenyl optionally substituted by: one, two or three groups independently selected from:
(a) —C₁₋₆alkyl, —C₁₋₆haloalkyl (such as —CF₃), —C₃₋₆cycloalkyl, —C₁₋₆alkoxy, —OR³, —CN, halogen or
(b) phenyl optionally substituted by one, two or three groups independently selected from: —C₁₋₆alkyl, —OR³, —C₁₋₆alkoxy —C₁₋₆haloalkyl (such as —CF₃), —CN or halogen.

In another aspect of the invention, R¹ represents phenyl optionally substituted by: one, two or three groups independently selected from:
(a) —C₁₋₃alkyl (such as —CH₃), —C₁₋₃haloalkyl (such as —CF₃), —C₃₋₆cycloalkyl, —C₁₋₃alkoxy (such as —OCH₃), —OR³, —CN or halogen (such as chloro, bromo or fluoro),
(b) or phenyl optionally substituted by one, two or three groups independently selected from: —C₁₋₃alkyl (such as —CH₃), —OR³, —C₁₋₃alkoxy (such as —OCH₃), —C₁₋₃haloalkyl (such as —CF₃), —CN or halogen (such as chloro, bromo or fluoro).

In another aspect of the invention, R¹ represents phenyl optionally substituted by: one or two groups independently selected from:
(a) —C₁₋₃alkyl (such as —CH₃), —C₁₋₃haloalkyl (such as —CF₃), —C₃₋₆cycloalkyl, —C₁₋₃alkoxy (such as —OCH₃), —OR³, —CN or halogen (such as chloro, bromo or fluoro),
(b) or phenyl optionally substituted by one, two or three groups independently selected from: —C₁₋₃alkyl (such as —CH₃), —OR³, —C₁₋₃alkoxy (such as —OCH₃), —C₁₋₃haloalkyl (such as —CF₃), —CN or halogen (such as chloro, bromo or fluoro).

In another aspect of the invention, R¹ represents phenyl optionally substituted by one, two or three groups independently selected from:
(a) —C₁₋₆alkyl (such as —CH₃), —C₁₋₆haloalkyl (such as —CF₃), —C₁₋₆alkoxy (such as —OCH₃), —OC₁₋₆haloalkyl (such as —OCF₃), —CN or halogen (such as chloro, bromo or fluoro) or
(b) phenyl optionally substituted by one, two or three groups independently selected from: halogen (such as chloro), —CN or —CF₃.

In another aspect of the invention, R¹ represents phenyl optionally substituted by one, two or three groups independently selected from:
(a) —C₁₋₆alkyl, —C₁₋₆haloalkyl (such as —CF₃), —CN, halogen (such as chloro, bromo or fluoro) or
(b) phenyl optionally substituted by one, two or three groups independently selected from: halogen (such as chloro), —CN or CF₃.

In another aspect of the invention, R¹ represents phenyl optionally substituted by one, two or three groups independently selected from:
(a) —C₁₋₆alkyl (such as —CH₃), —C₁₋₆haloalkyl (such as —CF₃), —C₁₋₆alkoxy (such as —OCH₃), —OC₁₋₆haloalkyl (such as —OCF₃), halogen (such as chloro, bromo or fluoro) or
(b) phenyl optionally substituted by one, two or three groups independently selected from: halogen (such as chloro).

In another aspect of the invention, R¹ represents phenyl optionally substituted by one, two or three groups independently selected from:
(a) —C₁₋₃alkyl (such as —CH₃), —C₁₋₃haloalkyl (such as —CF₃), —C₁₋₃alkoxy (such as —OCH₃), —OC₁₋₃haloalkyl (such as —OCF₃), halogen (such as chloro, bromo or fluoro) or
(b) phenyl optionally substituted by one, two or three groups independently selected from: halogen (such as chloro).

In another aspect of the invention, R¹ represents phenyl optionally substituted by one, two or three groups independently selected from:
(a) —C₁₋₆haloalkyl (such as —CF₃), halogen (such as chloro, bromo or fluoro) or
(b) phenyl optionally substituted by one, two or three groups independently selected from: halogen (such as chloro).

In another aspect of the invention, R¹ represents phenyl optionally substituted by one, two or three groups independently selected from: —CH₃, —OCH₃, —OCH₂CH(CH₃)₂, —CF₃, —OCF₃ or halogen (such as chloro, bromo or fluoro).

In another aspect of the invention, R¹ represents phenyl optionally substituted by one or two groups independently selected from: —CF₃, —CH₃, —OCH₃, —OCH₂CH(CH₃)₂, —CF₃, —OCF₃ or halogen (such as chloro, bromo or fluoro).

In another aspect of the invention, R¹ represents phenyl optionally substituted by one, two or three groups independently selected from: —CF₃ or halogen (such as chloro, bromo or fluoro).

In another aspect of the invention, R¹ represents phenyl optionally substituted by one or two groups independently selected from: —CF₃ or halogen (such as chloro, bromo or fluoro).

In another aspect of the invention, R¹ represents phenyl optionally substituted by one group independently selected from: —CF₃, —CH₃, —OCH₃, —OCH₂CH(CH₃)₂, —CF₃, —OCF₃ or halogen (such as chloro, bromo or fluoro).

In another aspect of the invention, R¹ represents phenyl substituted by phenyl, such as 2-phenyl, the second phenyl ring being optionally substituted by halogen (for example chloro).

In another aspect of the invention, R¹ represents phenyl substituted by phenyl.

In another aspect of the invention, R¹ represents naphthyl optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy(such as —OCH₃), —OR³, —CN or halogen (such as chloro, bromo or fluoro) or
(b) phenyl optionally substituted by one, two or three groups selected from —C₁₋₆alkyl (such as —CH₃), —OR³, —C₁₋₆alkoxy (such as —OCH₃), —C₁₋₆haloalkyl (such as —CF₃), —CN or halogen (such as chloro, bromo or fluoro).

In another aspect of the invention, R¹ represents naphthyl optionally substituted by: one, two or three groups independently selected from:
(a) —C₁₋₆alkyl, —C₁₋₆haloalkyl (such as —CF₃), —C₃₋₆cycloalkyl, —C₁₋₆alkoxy, —OR³, —CN, halogen or
(b) phenyl optionally substituted by one, two or three groups selected from: —C₁₋₆alkyl, —OR³, —C₁₋₆alkoxy, —C₁₋₆haloalkyl (such as —CF₃), —CN or halogen.

In another aspect of the invention, R¹ represents naphthyl.

In another aspect of the invention, R¹ represents tetrahydronaphthalenyl optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro), or
(b) phenyl optionally substituted by one, two or three groups selected from: —C₁₋₆alkyl (such as —CH₃), —C₁₋₆haloalkyl (such as —CF₃), —C₁₋₆alkoxy (such as —OCH₃), —OR³, —CN or halogen (such as chloro, bromo or fluoro).

In another aspect of the invention, R¹ represents tetrahydronaphthalenyl.

In another aspect of the invention, R¹ represents —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro),
(b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl (such as —CH₃), —C₁₋₆haloalkyl (such as —CF₃), —C₁₋₆alkoxy (such as —OCH₃), —OR³, —CN or halogen (such as chloro, bromo or fluoro).

In another aspect of the invention, R¹ represents —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
(a) —C₁₋₆alkyl, —C₁₋₆haloalkyl (such as —CF₃), —C₃₋₆cycloalkyl, —C₁₋₆alkoxy, —OR³, —CN or halogen (such as chloro, bromo or fluoro),
(b) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl, —OR³, —C₁₋₆alkoxy, —C₁₋₆haloalkyl (such as —CF₃), —CN or halogen (such as chloro, bromo or fluoro).

In another aspect of the invention, R¹ represents a —C₅₋₁₀heteroaryl. In another aspect of the invention, R¹ represents a —C₆heteroaryl. In another aspect of the invention, R¹ represents pyridine. In another aspect of the invention, R¹ represents a —C₅heteroaryl. In another aspect of the invention, R¹ represents thiophene.

In another aspect of the invention, R¹ represents a —C₅₋₁₀heteroaryl. In another aspect of the invention, R¹ represents a —C₈heteroaryl. In another aspect of the invention, R¹ represents benzothiophene. In another aspect of the invention, R¹ represents indole. In another aspect of the invention, R¹ represents N-methyl indole.

In another aspect of the invention, R¹ represents dihydro-2H-chromene optionally substituted by: one, two or three groups independently selected from:
(a) —C₁₋₆alkyl (such as —CH₃), —C₁₋₆haloalkyl (such as —CF₃), —C₃₋₆cycloalkyl, —C₁₋₆alkoxy (such as —OCH₃), —OR³, —CN or halogen (such as chloro, bromo or fluoro) or
(b) phenyl optionally substituted by one, two or three groups selected from: —C₁₋₆alkyl (such as —CH₃), —C₁₋₆haloalkyl (such as —CF₃), —C₁₋₆alkoxy (such as —OCH₃), —OR³, —CN or halogen (such as chloro, bromo or fluoro).

In another aspect of the invention, R¹ represents dihydro-2H-chromene.

In one aspect of the invention, Y represents —(CH₂)ₘ—, —O(CH₂)ₘ or —NR⁷(CH₂)ₘ—. In another aspect of the invention, Y represents —(CH₂)ₘ— or —O(CH₂)ₘ—. In another aspect of the invention, Y represents —(CH₂)ₘ—. In another aspect of the invention, Y represents —O(CH₂)ₘ—. In another aspect of the invention, Y represents —CH₂—, —OCH₂—, —OCH₂CH₂—, —C₂H₄— or —N(CH₃)CH₂—. In another aspect of the invention, Y represents —CH₂—, —OCH₂—, —OCH₂CH₂— or —C₂H₄—. In another aspect of the invention, Y represents —CH₂— (methylene). In another aspect of the invention, Y represents —C₂H₄— (ethylene). In another aspect of the invention, Y represents —OCH₂—. In another aspect of the invention, Y represents —OCH₂CH₂—. In another aspect of the invention, Y represents —N(CH₃)CH₂—.

In one aspect of the invention, R² represents hydrogen. In another aspect of the invention, R² represents —C₃H₇. In another aspect of the invention, R² represents —C(═O)C₁₋₆alkyl or —C(═O)C₆₋₁₀aryl. In another aspect of the invention, R² represents —C(═O)phenyl. In another aspect of the invention, R² represents —C(═O)C₃H₇. In another aspect of the invention, R² represents —C(═O)C₆H₅. In another aspect of the invention, R² represents —C(═O)C₁₋₆alkylOH. In another aspect of the invention, R² represents —C(═O)CH₂OH. In another aspect of the invention, R² represents —C(═O)C(CH₃)₂OH. In another aspect of the invention, R² represents —COCH₂N(CH₃)₂.

In another aspect of the invention, R² represents thiazoleCH₂OH.

In one aspect of the invention, R³ represents —OC₁₋₆haloalkyl (such as —OCF₃). In another aspect of the invention, R³ represents —OC₁₋₃haloalkyl (such as —OCF₃). In another aspect of the invention, R³ represents —OC₃₋₆cycloalkyl.

In one aspect of the invention, R⁴ represents —C₁₋₃alkyl (such as —CH₃). In another aspect of the invention, R⁴ represents —CH₃ (methyl). In another aspect of the invention, R⁴ represents hydrogen.

In one aspect of the invention, R⁵ represents —C₁₋₃alkyl (such as —CH₃). In another aspect of the invention, R⁵ represents —CH₃ (methyl). In another aspect of the invention, R⁵ represents hydrogen.

In one aspect of the invention, R⁷ represents —C₁₋₃alkyl (such as —CH₃). In another aspect of the invention, R⁷ represents —CH₃ (methyl). In another aspect of the invention, R⁷ represents hydrogen.

In one aspect of the invention, m represents 0, 1, 2 or 3. In another aspect of the invention, m represents 1 or 2.

Each of the aspects of the invention are independent unless stated otherwise. Nevertheless the skilled person will understand that all the permutations of the aspects of described are within the scope of the invention. Thus it is to be understood that the present invention covers all combinations of suitable, convenient and exemplified groups described herein. For example, in one aspect the invention provides a compound of formula (I) wherein X represents —NHCO— and R² represents H.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The invention also extends to conformational isomers of compounds of formula (I) and any geometric (cis and/or trans) isomers of said compounds. Likewise, it is understood that compounds of formula (I) may exist in tautomeric forms other than that shown in the formula and these are also included within the scope of the present invention.

It will be appreciated that racemic compounds of formula (I) may be optionally resolved into their individual enantiomers. Such resolutions may conveniently be accomplished by standard methods known in the art. For example, a racemic compound of formula (I) may be resolved by chiral preparative HPLC.

It will also be appreciated that compounds of the invention which exist as polymorphs, and mixtures thereof, are within the scope of the present invention.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl. However, when a moiety is defined such that alkyl bears a substituent it will be clear to the skilled person from the context that alkyl may include alkylene, for example methylene (—CH₂—), ethylene (—CH₂CH₂—) and propylene (—CH₂CH₂CH₂—).

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. The point of attachment may be on the oxygen or carbon atom.

As used herein, the term “halogen” or “halo” refers to a fluorine (fluoro), chlorine (chloro), bromine (bromo) or iodine (iodo) atom.

As used herein, the term “haloalkyl” refers to an alkyl group having one or more carbon atoms and wherein at least one hydrogen atom is replaced with a halogen atom, for example a trifluoromethyl group and the like.

As used herein, the term “cycloalkyl” refers to a saturated cyclic group containing 3 to 10 carbon ring-atoms, such as 3 to 6 carbon ring-atoms. Examples include cyclopropyl, cyclopentyl and cyclohexyl.

As used herein, the term “C₅₋₁₀heteroaryl” refers to an aromatic cyclic group containing 5 to 10 ring-atoms 1, 2, 3 or 4 of which are hetero-atoms independently selected from nitrogen, oxygen and sulphur and the remaining ring-atoms are carbon, e.g. benzothiophenyl, indolyl or thienyl. This definition includes both monocyclic and bicyclic ring systems and bicyclic structures at least a portion of which is aromatic and the other part is saturated, partially or fully unsaturated.

As used herein, the term ‘aryl’ means an aromatic carbocyclic moiety. The definition includes both monocyclic and bicyclic ring systems and bicyclic structures at least a portion of which is aromatic and the other part is saturated, partially or fully unsaturated. Examples of aromatic, aryl groups include naphthyl, anthryl, phenanthryl, indanyl, indenyl, azulenyl, azulanyl, fluorenyl, phenyl and napthyl, and more specifically phenyl.

As used herein, the term “C₅₋₁₀heterocyclyl” refers to a cyclic group containing 5 to 10 ring-atoms 1, 2, 3 or 4 of which are hetero-atoms independently selected from nitrogen, oxygen and sulphur and the remaining ring-atoms are carbon, wherein said cyclic group is saturated, partially or fully unsaturated but, which is not aromatic e.g. tetrahydrofuran, dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, 1,3-dioxane, 1,3-dithiane, oxathiane or thiomorpholine. This definition includes bicyclic structures provided the moiety is non-aromatic.

Examples of heterocyclyl and heteroaryl groups include: furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, homopiperazinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl, thiazepinyl, diazepinyl and thiazolinyl, benzimidazolyl, benzoxazolyl, imidazopyridinyl, benzoxazinyl, benzothiazinyl, benzothiophenyl oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl and isoindolyl.

As used herein, the term “substituted” refers to substitution with the named substituent or substituents, multiple degrees of substitution being allowed unless otherwise stated.

For the avoidance of doubt, the term “independently” means that where more than one substituent is selected from a number of possible substituents, those substituents may be the same or different.

As used herein, the term “pharmaceutically acceptable” means a compound which is suitable for pharmaceutical use.

Salts of compounds of formula (I) which are suitable for use in medicine are those wherein the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts.

Suitable pharmaceutically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic, ethanesulfonic or naphthalenesulfonic acid. Other non-pharmaceutically acceptable salts e.g. oxalates, may be used, for example in the isolation of compounds of formula (I) and are included within the scope of this invention. Reference is made to Berge et al. J. Pharm. Sci., 1977, 66, 1-19.

Certain of the compounds of formula (I) may form acid addition salts with one or more equivalents of the acid. The present invention includes within its scope all possible stoichiometric and non-stoichiometric forms thereof.

Solvates of the compounds of formula (I) and solvates of the salts of the compounds of formula (I) are included within the scope of the present invention.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include but are not limited to, water, methanol, ethanol and acetic acid. Preferably the solvent used is a pharmaceutically acceptable solvent. Most preferably the solvent used is water and the solvate may also be referred to as a hydrate.

Solvates of compounds of formula (I) which are suitable for use in medicine are those wherein the solvent is pharmaceutically acceptable. However, solvates having non-pharmaceutically acceptable solvents are within the scope of the present invention, for example, for use as intermediates in the preparation of other compounds of formula (I) and their pharmaceutically acceptable salts.

Prodrugs of the compounds of formula (I) are included with the scope of the present invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987 and in D. Fleishner, S. Ramon and H. Barba “Improved oral drug delivery: solubility limitations overcome by the use of prodrugs”, Advanced Drug Delivery Reviews (1996) 19(2) 115-130. Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein hydroxyl or amine groups are bonded to any group that, when administered to a patient, cleaves to form the hydroxy or amine groups. Thus, representative examples of prodrugs include (but are not limited to) phosphonate, carbamate, acetate, formate and benzoate derivatives of hydroxy and amine functional groups of the compounds of formula (I).

Phosphonates and carbamates may be active in their own right and/or be hydrolysable under in vivo conditions in the human body. Suitable pharmaceutically acceptable in vivo hydrolysable ester groups include those which break down readily in the human body to leave the parent acid or its salt. A phosphonate is formed by reaction with phosphorous (phosphonic) acid, by methods well known in the art. For example, phosphonates may be derivatives such as RP(O)(OR)₂ and the like. A carbamate is an ester of carbamic acid.

In one aspect of the invention there is provided a compound, or a pharmaceutically acceptable salt thereof, wherein the compound is selected from the group consisting of:
N-(5-{[(2-chlorophenyl)oxy]methyl}-1,3,4-thiadiazol-2-yl)-1,2,3,4-tetrahydro-6-isoquinolinecarboxamide,
N-[5-(1-naphthalenylmethyl)-1,3,4-thiadiazol-2-yl]-1,2,3,4-tetrahydro-6-isoquinolinecarboxamide,
N-(5-{[(3,4-dichlorophenyl)oxy]methyl}-1,3,4-thiadiazol-2-yl)-1,2,3,4-tetrahydro-6-isoquinolinecarboxamide,
N-(5-{[(4-chlorophenyl)oxy]methyl}-1,3,4-thiadiazol-2-yl)-1,2,3,4-tetrahydro-6-isoquinolinecarboxamide,
N-[5-(phenylmethyl)-1,3,4-thiadiazol-2-yl]-1,2,3,4-tetrahydro-6-isoquinolinecarboxamide,
N-{5-[(4-chlorophenyl)methyl]-1,3,4-thiadiazol-2-yl}-1,2,3,4-tetrahydro-6-isoquinolinecarboxamide,
N-{5-[(3,4-dichlorophenyl)methyl]-1,3,4-thiadiazol-2-yl}-1,2,3,4-tetrahydro-6-isoquinolinecarboxamide,
N-[5-(2-thienylmethyl)-1,3,4-thiadiazol-2-yl]-1,2,3,4-tetrahydro-6-isoquinolinecarboxamide,
N-[5-(2-naphthalenylmethyl)-1,3,4-thiadiazol-2-yl]-1,2,3,4-tetrahydro-6-isoquinolinecarboxamide,
N-[5-(cyclohexylmethyl)-1,3,4-thiadiazol-2-yl]-1,2,3,4-tetrahydro-6-isoquinolinecarboxamide,
N-[5-(2-phenylethyl)-1,3,4-thiadiazol-2-yl]-1,2,3,4-tetrahydro-6-isoquinolinecarboxamide,
N-[5-(1H-indol-3-ylmethyl)-1,3,4-thiadiazol-2-yl]-1,2,3,4-tetrahydro-6-isoquinolinecarboxamide,
N-(5-{[(2,5-dichlorophenyl)oxy]methyl}-1,3,4-thiadiazol-2-yl)-1,2,3,4-tetrahydro-6-isoquinolinecarboxamide,
N-{5-[(1-naphthalenyloxy)methyl]-1,3,4-thiadiazol-2-yl}-1,2,3,4-tetrahydro-6-isoquinolinecarboxamide,
N-(5-{[(2-chloro-4-fluorophenyl)oxy]methyl}-1,3,4-thiadiazol-2-yl)-1,2,3,4-tetrahydro-6-isoquinolinecarboxamide,
N-(5-{[(2-chloro-5-fluorophenyl)oxy]methyl}-1,3,4-thiadiazol-2-yl)-1,2,3,4-tetrahydro-6-isoquinolinecarboxamide,
N-[5-(1-benzothien-3-ylmethyl)-1,3,4-thiadiazol-2-yl]-1,2,3,4-tetrahydro-6-isoquinolinecarboxamide,
N-[5-(3-thienylmethyl)-1,3,4-thiadiazol-2-yl]-1,2,3,4-tetrahydro-6-isoquinolinecarboxamide,
N-{5-[2-(1-naphthalenyl)ethyl]-1,3,4-thiadiazol-2-yl}-1,2,3,4-tetrahydro-6-isoquinolinecarboxamide,
N-{5-[2-(2-chlorophenyl)ethyl]-1,3,4-thiadiazol-2-yl}-1,2,3,4-tetrahydro-6-isoquinolinecarboxamide,
N-{5-[(2-bromophenyl)methyl]-1,3,4-thiadiazol-2-yl}-1,2,3,4-tetrahydro-6-isoquinolinecarboxamide,
N-(5-{[(2-fluorophenyl)oxy]methyl}-1,3,4-thiadiazol-2-yl)-1,2,3,4-tetrahydro-6-isoquinolinecarboxamide,
N-(5-{[(3-fluorophenyl)oxy]methyl}-1,3,4-thiadiazol-2-yl)-1,2,3,4-tetrahydro-6-isoquinolinecarboxamide,
N-(5-{[(4-fluorophenyl)oxy]methyl}-1,3,4-thiadiazol-2-yl)-1,2,3,4-tetrahydro-6-isoquinolinecarboxamide,
N-(5-{[(3-chlorophenyl)oxy]methyl}-1,3,4-thiadiazol-2-yl)-1,2,3,4-tetrahydro-6-isoquinolinecarboxamide,
N-[5-({[2-(trifluoromethyl)phenyl]oxy}methyl)-1,3,4-thiadiazol-2-yl]-1,2,3,4-tetrahydro-6-isoquinolinecarboxamide,
N-[5-({[3-(trifluoromethyl)phenyl]oxy}methyl)-1,3,4-thiadiazol-2-yl]-1,2,3,4-tetrahydro-6-isoquinolinecarboxamide,
N-(5-{[3-(trifluoromethyl)phenyl]methyl}-1,3,4-thiadiazol-2-yl)-1,2,3,4-tetrahydro-6-isoquinolinecarboxamide,
N-{5-[(5,6,7,8-tetrahydro-1-naphthalenyloxy)methyl]-1,3,4-thiadiazol-2-yl}-1,2,3,4-tetrahydro-6-isoquinolinecarboxamide,
N-{5-[(2-chlorophenyl)methyl]-1,3,4-thiadiazol-2-yl}-1,2,3,4-tetrahydro-6-isoquinolinecarboxamide,
N-(5-{[2-(trifluoromethyl)phenyl]methyl}-1,3,4-thiadiazol-2-yl)-1,2,3,4-tetrahydro-6-isoquinolinecarboxamide,
N-[5-({[4-(methyloxy)phenyl]oxy}methyl)-1,3,4-thiadiazol-2-yl]-1,2,3,4-tetrahydro-6-isoquinolinecarboxamide,
N-{5-[(2-biphenylyloxy)methyl]-1,3,4-thiadiazol-2-yl}-1,2,3,4-tetrahydro-6-isoquinolinecarboxamide,
N-(5-{[4-(trifluoromethyl)phenyl]methyl}-1,3,4-thiadiazol-2-yl)-1,2,3,4-tetrahydro-6-isoquinolinecarboxamide,
N-{5-[({5-chloro-2-[(2-methylpropyl)oxy]phenyl}oxy)methyl]-1,3,4-thiadiazol-2-yl}-1,2,3,4-tetrahydro-6-isoquinolinecarboxamide,
N-{5-[(4-fluorophenyl)methyl]-1,3,4-thiadiazol-2-yl}-1,2,3,4-tetrahydro-6-isoquinolinecarboxamide,
N-[5-({[2-(methyloxy)phenyl]oxy}methyl)-1,3,4-thiadiazol-2-yl]-1,2,3,4-tetrahydro-6-isoquinolinecarboxamide,
N-{5-[(1-methyl-1H-indol-3-yl)methyl]-1,3,4-thiadiazol-2-yl}-1,2,3,4-tetrahydro-6-isoquinolinecarboxamide,
N-[5-(3-pyridinylmethyl)-1,3,4-thiadiazol-2-yl]-1,2,3,4-tetrahydro-6-isoquinolinecarboxamide,
N-[5-(5,6,7,8-tetrahydro-2-naphthalenylmethyl)-1,3,4-thiadiazol-2-yl]-1,2,3,4-tetrahydro-6-isoquinolinecarboxamide,
N-[5-(3,4-dihydro-2H-chromen-6-ylmethyl)-1,3,4-thiadiazol-2-yl]-1,2,3,4-tetrahydro-6-isoquinolinecarboxamide,
N-(5-{2-[(2-chlorophenyl)oxy]ethyl}-1,3,4-thiadiazol-2-yl)-1,2,3,4-tetrahydro-6-isoquinolinecarboxamide,
N-(5-{[(2,4-dichlorophenyl)oxy]methyl}-1,3,4-thiadiazol-2-yl)-1,2,3,4-tetrahydro-6-isoquinolinecarboxamide,
N-{5-[(2′-chloro-2-biphenylyl)methyl]-1,3,4-thiadiazol-2-yl}-1,2,3,4-tetrahydro-6-isoquinolinecarboxamide,
N-{5-[(2-fluorophenyl)methyl]-1,3,4-thiadiazol-2-yl}-1,2,3,4-tetrahydro-6-isoquinolinecarboxamide,
N-{5-[(3-chlorophenyl)methyl]-1,3,4-thiadiazol-2-yl}-1,2,3,4-tetrahydro-6-isoquinolinecarboxamide,
N-(5-{[(2,6-dichlorophenyl)oxy]methyl}-1,3,4-thiadiazol-2-yl)-1,2,3,4-tetrahydro-6-isoquinolinecarboxamide,
N-(5-{[(2-methylphenyl)oxy]methyl}-1,3,4-thiadiazol-2-yl)-1,2,3,4-tetrahydro-6-isoquinolinecarboxamide,
N-(5-{[(3,4-dimethylphenyl)oxy]methyl}-1,3,4-thiadiazol-2-yl)-1,2,3,4-tetrahydro-6-isoquinolinecarboxamide,
N-{5-[(2,4-dichlorophenyl)methyl]-1,3,4-thiadiazol-2-yl}-1,2,3,4-tetrahydro-6-isoquinolinecarboxamide,
N-[5-({2-[(trifluoromethyl)oxy]phenyl}methyl)-1,3,4-thiadiazol-2-yl]-1,2,3,4-tetrahydro-6-isoquinolinecarboxamide,
N-(5-{[(2-chloro-3,5-difluorophenyl)oxy]methyl}-1,3,4-thiadiazol-2-yl)-1,2,3,4-tetrahydro-6-isoquinolinecarboxamide,
N-(5-{[(2-chloro-6-fluorophenyl)oxy]methyl}-1,3,4-thiadiazol-2-yl)-1,2,3,4-tetrahydro-6-isoquinolinecarboxamide,
N-[5-({[2-chloro-3-(trifluoromethyl)phenyl]oxy}methyl)-1,3,4-thiadiazol-2-yl]-1,2,3,4-tetrahydro-6-isoquinolinecarboxamide,
N-(5-{[(2,4,5-trichlorophenyl)oxy]methyl}-1,3,4-thiadiazol-2-yl)-1,2,3,4-tetrahydro-6-isoquinolinecarboxamide,
N-[5-({[2-chloro-5-(trifluoromethyl)phenyl]oxy}methyl)-1,3,4-thiadiazol-2-yl]-1,2,3,4-tetrahydro-6-isoquinolinecarboxamide,
N-{5-[(4-chloro-2-fluorophenyl)methyl]-1,3,4-thiadiazol-2-yl}-1,2,3,4-tetrahydro-6-isoquinolinecarboxamide,
N-(5-{[4-fluoro-2-(trifluoromethyl)phenyl]methyl}-1,3,4-thiadiazol-2-yl)-1,2,3,4-tetrahydro-6-isoquinolinecarboxamide,
N-(5-{[5-chloro-2-(trifluoromethyl)phenyl]methyl}-1,3,4-thiadiazol-2-yl)-1,2,3,4-tetrahydro-6-isoquinolinecarboxamide,
N-[5-({[4-fluoro-2-(trifluoromethyl)phenyl]oxy}methyl)-1,3,4-thiadiazol-2-yl]-1,2,3,4-tetrahydro-6-isoquinolinecarboxamide,
N-[5-({[2-chloro-4-(trifluoromethyl)phenyl]oxy}methyl)-1,3,4-thiadiazol-2-yl]-1,2,3,4-tetrahydro-6-isoquinolinecarboxamide,
N-(5-{[(3-chloro-5-fluorophenyl)oxy]methyl}-1,3,4-thiadiazol-2-yl)-1,2,3,4-tetrahydro-6-isoquinolinecarboxamide,
N-[5-({[5-fluoro-2-(trifluoromethyl)phenyl]oxy}methyl)-1,3,4-thiadiazol-2-yl]-1,2,3,4-tetrahydro-6-isoquinolinecarboxamide,
N-(5-{[(2,4-difluorophenyl)oxy]methyl}-1,3,4-thiadiazol-2-yl)-1,2,3,4-tetrahydro-6-isoquinolinecarboxamide,
5-{[(2-chlorophenyl)oxy]methyl}-N-(1,2,3,4-tetrahydro-6-isoquinolinyl)-1,3,4-thiadiazole-2-carboxamide,
N-(5-{[(2-chlorophenyl)(methyl)amino]methyl}-1,3,4-thiadiazol-2-yl)-1,2,3,4-tetrahydro-6-isoquinolinecarboxamide,
N-(5-{[(2-chlorophenyl)oxy]methyl}-1,3,4-thiadiazol-2-yl)-N-methyl-1,2,3,4-tetrahydro-6-isoquinolinecarboxamide,
N-(5-{[(2-chlorophenyl)oxy]methyl}-1,3,4-thiadiazol-2-yl)-2-(hydroxyacetyl)-1,2,3,4-tetrahydro-6-isoquinolinecarboxamide,
N-(5-{[(2-chlorophenyl)oxy]methyl}-1,3,4-thiadiazol-2-yl)-2-(2-hydroxy-2-methylpropanoyl)-1,2,3,4-tetrahydro-6-isoquinolinecarboxamide,
N-(5-{[(2-chlorophenyl)oxy]methyl}-1,3,4-thiadiazol-2-yl)-2-(N,N-dimethylglycyl)-1,2,3,4-tetrahydro-6-isoquinolinecarboxamide,
N-(5-{[(2-chlorophenyl)oxy]methyl}-1,3,4-thiadiazol-2-yl)-2-(phenylcarbonyl)-1,2,3,4-tetrahydro-6-isoquinolinecarboxamide,
2-butanoyl-N-(5-{[(2-chlorophenyl)oxy]methyl}-1,3,4-thiadiazol-2-yl)-1,2,3,4-tetrahydro-6-isoquinolinecarboxamide,
N-(5-{[(2-chlorophenyl)oxy]methyl}-1,3,4-thiadiazol-2-yl)-2-propyl-1,2,3,4-tetrahydro-6-isoquinolinecarboxamide,
N-(5-{[(2-chlorophenyl)oxy]methyl}-1,3,4-thiadiazol-2-yl)-2-[5-(hydroxymethyl)-1,3-thiazol-2-yl]-1,2,3,4-tetrahydro-6-isoquinolinecarboxamide, or
5-[(3,4-dichlorophenyl)methyl]-N-(1,2,3,4-tetrahydro-6-isoquinolinyl)-1,3,4-thiadiazole-2-carboxamide.

In another aspect of the invention there is provided a compound, selected from the group consisting of:
N-(5-{[(2-chlorophenyl)oxy]methyl}-1,3,4-thiadiazol-2-yl)-1,2,3,4-tetrahydro-6-isoquinolinecarboxamide hydrochloride,
N-[5-(1-naphthalenylmethyl)-1,3,4-thiadiazol-2-yl]-1,2,3,4-tetrahydro-6-isoquinolinecarboxamide hydrochloride,
N-(5-{[(3,4-dichlorophenyl)oxy]methyl}-1,3,4-thiadiazol-2-yl)-1,2,3,4-tetrahydro-6-isoquinolinecarboxamide hydrochloride,
N-(5-{[(4-chlorophenyl)oxy]methyl}-1,3,4-thiadiazol-2-yl)-1,2,3,4-tetrahydro-6-isoquinolinecarboxamide hydrochloride,
N-[5-(phenylmethyl)-1,3,4-thiadiazol-2-yl]-1,2,3,4-tetrahydro-6-isoquinolinecarboxamide hydrochloride,
N-{5-[(4-chlorophenyl)methyl]-1,3,4-thiadiazol-2-yl}-1,2,3,4-tetrahydro-6-isoquinolinecarboxamide hydrochloride,
N-{5-[(3,4-dichlorophenyl)methyl]-1,3,4-thiadiazol-2-yl}-1,2,3,4-tetrahydro-6-isoquinolinecarboxamide hydrochloride,
N-[5-(2-thienylmethyl)-1,3,4-thiadiazol-2-yl]-1,2,3,4-tetrahydro-6-isoquinolinecarboxamide hydrochloride,
N-[5-(2-naphthalenylmethyl)-1,3,4-thiadiazol-2-yl]-1,2,3,4-tetrahydro-6-isoquinolinecarboxamide hydrochloride,
N-[5-(cyclohexylmethyl)-1,3,4-thiadiazol-2-yl]-1,2,3,4-tetrahydro-6-isoquinolinecarboxamide hydrochloride,
N-[5-(2-phenylethyl)-1,3,4-thiadiazol-2-yl]-1,2,3,4-tetrahydro-6-isoquinolinecarboxamide hydrochloride,
N-[5-(1H-indol-3-ylmethyl)-1,3,4-thiadiazol-2-yl]-1,2,3,4-tetrahydro-6-isoquinolinecarboxamide hydrochloride,
N-(5-{[(2,5-dichlorophenyl)oxy]methyl}-1,3,4-thiadiazol-2-yl)-1,2,3,4-tetrahydro-6-isoquinolinecarboxamide hydrochloride,
N-{5-[(1-naphthalenyloxy)methyl]-1,3,4-thiadiazol-2-yl}-1,2,3,4-tetrahydro-6-isoquinolinecarboxamide hydrochloride,
N-(5-{[(2-chloro-4-fluorophenyl)oxy]methyl}-1,3,4-thiadiazol-2-yl)-1,2,3,4-tetrahydro-6-isoquinolinecarboxamide hydrochloride,
N-(5-{[(2-chloro-5-fluorophenyl)oxy]methyl}-1,3,4-thiadiazol-2-yl)-1,2,3,4-tetrahydro-6-isoquinolinecarboxamide hydrochloride,
N-[5-(1-benzothien-3-ylmethyl)-1,3,4-thiadiazol-2-yl]-1,2,3,4-tetrahydro-6-isoquinolinecarboxamide hydrochloride,
N-[5-(3-thienylmethyl)-1,3,4-thiadiazol-2-yl]-1,2,3,4-tetrahydro-6-isoquinolinecarboxamide hydrochloride,
N-{5-[2-(1-naphthalenyl)ethyl]-1,3,4-thiadiazol-2-yl}-1,2,3,4-tetrahydro-6-isoquinolinecarboxamide hydrochloride,
N-{5-[2-(2-chlorophenyl)ethyl]-1,3,4-thiadiazol-2-yl}-1,2,3,4-tetrahydro-6-isoquinolinecarboxamide hydrochloride,
N-{5-[(2-bromophenyl)methyl]-1,3,4-thiadiazol-2-yl}-1,2,3,4-tetrahydro-6-isoquinolinecarboxamide hydrochloride,
N-(5-{[(2-fluorophenyl)oxy]methyl}-1,3,4-thiadiazol-2-yl)-1,2,3,4-tetrahydro-6-isoquinolinecarboxamide hydrochloride,
N-(5-{[(3-fluorophenyl)oxy]methyl}-1,3,4-thiadiazol-2-yl)-1,2,3,4-tetrahydro-6-isoquinolinecarboxamide hydrochloride,
N-(5-{[(4-fluorophenyl)oxy]methyl}-1,3,4-thiadiazol-2-yl)-1,2,3,4-tetrahydro-6-isoquinolinecarboxamide hydrochloride,
N-(5-{[(3-chlorophenyl)oxy]methyl}-1,3,4-thiadiazol-2-yl)-1,2,3,4-tetrahydro-6-isoquinolinecarboxamide hydrochloride,
N-[5-({[2-(trifluoromethyl)phenyl]oxy}methyl)-1,3,4-thiadiazol-2-yl]-1,2,3,4-tetrahydro-6-isoquinolinecarboxamide hydrochloride,
N-[5-({[3-(trifluoromethyl)phenyl]oxy}methyl)-1,3,4-thiadiazol-2-yl]-1,2,3,4-tetrahydro-6-isoquinolinecarboxamide hydrochloride,
N-(5-{[3-(trifluoromethyl)phenyl]methyl}-1,3,4-thiadiazol-2-yl)-1,2,3,4-tetrahydro-6-isoquinolinecarboxamide hydrochloride,
N-{5-[(5,6,7,8-tetrahydro-1-naphthalenyloxy)methyl]-1,3,4-thiadiazol-2-yl}-1,2,3,4-tetrahydro-6-isoquinolinecarboxamide hydrochloride,
N-{5-[(2-chlorophenyl)methyl]-1,3,4-thiadiazol-2-yl}-1,2,3,4-tetrahydro-6-isoquinolinecarboxamide hydrochloride,
N-(5-{[2-(trifluoromethyl)phenyl]methyl}-1,3,4-thiadiazol-2-yl)-1,2,3,4-tetrahydro-6-isoquinolinecarboxamide hydrochloride,
N-[5-({[4-(methyloxy)phenyl]oxy}methyl)-1,3,4-thiadiazol-2-yl]-1,2,3,4-tetrahydro-6-isoquinolinecarboxamide hydrochloride,
N-{5-[(2-biphenylyloxy)methyl]-1,3,4-thiadiazol-2-yl}-1,2,3,4-tetrahydro-6-isoquinolinecarboxamide hydrochloride,
N-(5-{[4-(trifluoromethyl)phenyl]methyl}-1,3,4-thiadiazol-2-yl)-1,2,3,4-tetrahydro-6-isoquinolinecarboxamide hydrochloride,
N-{5-[({5-chloro-2-[(2-methylpropyl)oxy]phenyl}oxy)methyl]-1,3,4-thiadiazol-2-yl}-1,2,3,4-tetrahydro-6-isoquinolinecarboxamide hydrochloride,
N-{5-[(4-fluorophenyl)methyl]-1,3,4-thiadiazol-2-yl}-1,2,3,4-tetrahydro-6-isoquinolinecarboxamide hydrochloride,
N-[5-({[2-(methyloxy)phenyl]oxy}methyl)-1,3,4-thiadiazol-2-yl]-1,2,3,4-tetrahydro-6-isoquinolinecarboxamide hydrochloride,
N-{5-[(1-methyl-1H-indol-3-yl)methyl]-1,3,4-thiadiazol-2-yl}-1,2,3,4-tetrahydro-6-isoquinolinecarboxamide hydrochloride,
N-[5-(3-pyridinylmethyl)-1,3,4-thiadiazol-2-yl]-1,2,3,4-tetrahydro-6-isoquinolinecarboxamide hydrochloride,
N-[5-(5,6,7,8-tetrahydro-2-naphthalenylmethyl)-1,3,4-thiadiazol-2-yl]-1,2,3,4-tetrahydro-6-isoquinolinecarboxamide hydrochloride,
N-[5-(3,4-dihydro-2H-chromen-6-ylmethyl)-1,3,4-thiadiazol-2-yl]-1,2,3,4-tetrahydro-6-isoquinolinecarboxamide hydrochloride,
N-(5-{2-[(2-chlorophenyl)oxy]ethyl}-1,3,4-thiadiazol-2-yl)-1,2,3,4-tetrahydro-6-isoquinolinecarboxamide hydrochloride,
N-(5-{[(2,4-dichlorophenyl)oxy]methyl}-1,3,4-thiadiazol-2-yl)-1,2,3,4-tetrahydro-6-isoquinolinecarboxamide hydrochloride,
N-{5-[(2′-chloro-2-biphenylypmethyl]-1,3,4-thiadiazol-2-yl}-1,2,3,4-tetrahydro-6-isoquinolinecarboxamide hydrochloride,
N-{5-[(2-fluorophenyl)methyl]-1,3,4-thiadiazol-2-yl}-1,2,3,4-tetrahydro-6-isoquinolinecarboxamide hydrochloride,
N-{5-[(3-chlorophenyl)methyl]-1,3,4-thiadiazol-2-yl}-1,2,3,4-tetrahydro-6-isoquinolinecarboxamide hydrochloride,
N-(5-{[(2,6-dichlorophenyl)oxy]methyl}-1,3,4-thiadiazol-2-yl)-1,2,3,4-tetrahydro-6-isoquinolinecarboxamide hydrochloride,
N-(5-{[(2-methylphenyl)oxy]methyl}-1,3,4-thiadiazol-2-yl)-1,2,3,4-tetrahydro-6-isoquinolinecarboxamide hydrochloride,
N-(5-{[(3,4-dimethylphenyl)oxy]methyl}-1,3,4-thiadiazol-2-yl)-1,2,3,4-tetrahydro-6-isoquinolinecarboxamide hydrochloride,
N-{5-[(2,4-dichlorophenyl)methyl]-1,3,4-thiadiazol-2-yl}-1,2,3,4-tetrahydro-6-isoquinolinecarboxamide hydrochloride,
N-[5-({2-[(trifluoromethyl)oxy]phenyl}methyl)-1,3,4-thiadiazol-2-yl]-1,2,3,4-tetrahydro-6-isoquinolinecarboxamide hydrochloride,
N-(5-{[(2-chloro-3,5-difluorophenyl)oxy]methyl}-1,3,4-thiadiazol-2-yl)-1,2,3,4-tetrahydro-6-isoquinolinecarboxamide hydrochloride,
N-(5-{[(2-chloro-6-fluorophenyl)oxy]methyl}-1,3,4-thiadiazol-2-yl)-1,2,3,4-tetrahydro-6-isoquinolinecarboxamide hydrochloride,
N-[5-({[2-chloro-3-(trifluoromethyl)phenyl]oxy}methyl)-1,3,4-thiadiazol-2-yl]-1,2,3,4-tetrahydro-6-isoquinolinecarboxamide hydrochloride,
N-(5-{[(2,4,5-trichlorophenyl)oxy]methyl}-1,3,4-thiadiazol-2-yl)-1,2,3,4-tetrahydro-6-isoquinolinecarboxamide hydrochloride,
N-[5-({[2-chloro-5-(trifluoromethyl)phenyl]oxy}methyl)-1,3,4-thiadiazol-2-yl]-1,2,3,4-tetrahydro-6-isoquinolinecarboxamide hydrochloride,
N-{5-[(4-chloro-2-fluorophenyl)methyl}-1,3,4-thiadiazol-2-yl]-1,2,3,4-tetrahydro-6-isoquinolinecarboxamide hydrochloride,
N-(5-{[4-fluoro-2-(trifluoromethyl)phenyl]methyl}-1,3,4-thiadiazol-2-yl)-1,2,3,4-tetrahydro-6-isoquinolinecarboxamide hydrochloride,
N-(5-{[5-chloro-2-(trifluoromethyl)phenyl]methyl}-1,3,4-thiadiazol-2-yl)-1,2,3,4-tetrahydro-6-isoquinolinecarboxamide hydrochloride,
N-[5-({[4-fluoro-2-(trifluoromethyl)phenyl]oxy}methyl)-1,3,4-thiadiazol-2-yl]-1,2,3,4-tetrahydro-6-isoquinolinecarboxamide hydrochloride,
N-[5-({[2-chloro-4-(trifluoromethyl)phenyl]oxy}methyl)-1,3,4-thiadiazol-2-yl]-1,2,3,4-tetrahydro-6-isoquinolinecarboxamide hydrochloride,
N-(5-{[(3-chloro-5-fluorophenyl)oxy]methyl}-1,3,4-thiadiazol-2-yl)-1,2,3,4-tetrahydro-6-isoquinolinecarboxamide hydrochloride,
N-[5-({[5-fluoro-2-(trifluoromethyl)phenyl]oxy}methyl)-1,3,4-thiadiazol-2-yl]-1,2,3,4-tetrahydro-6-isoquinolinecarboxamide hydrochloride,
N-(5-{[(2,4-difluorophenyl)oxy]methyl}-1,3,4-thiadiazol-2-yl)-1,2,3,4-tetrahydro-6-isoquinolinecarboxamide hydrochloride,
5-{[(2-chlorophenyl)oxy]methyl}-N-(1,2,3,4-tetrahydro-6-isoquinolinyl)-1,3,4-thiadiazole-2-carboxamide hydrochloride,
N-(5-{[(2-chlorophenyl)(methyl)amino]methyl}-1,3,4-thiadiazol-2-yl)-1,2,3,4-tetrahydro-6-isoquinolinecarboxamide hydrochloride,
N-(5-{[(2-chlorophenyl)oxy]methyl}-1,3,4-thiadiazol-2-yl)-N-methyl-1,2,3,4-tetrahydro-6-isoquinolinecarboxamide hydrochloride,
N-(5-{[(2-chlorophenyl)oxy]methyl}-1,3,4-thiadiazol-2-yl)-2-(hydroxyacetyl)-1,2,3,4-tetrahydro-6-isoquinolinecarboxamide,
N-(5-{[(2-chlorophenyl)oxy]methyl}-1,3,4-thiadiazol-2-yl)-2-(2-hydroxy-2-methylpropanoyl)-1,2,3,4-tetrahydro-6-isoquinolinecarboxamide,
N-(5-{[(2-chlorophenyl)oxy]methyl}-1,3,4-thiadiazol-2-yl)-2-(N,N-dimethylglycyl)-1,2,3,4-tetrahydro-6-isoquinolinecarboxamide,
N-(5-{[(2-chlorophenyl)oxy]methyl}-1,3,4-thiadiazol-2-yl)-2-(phenylcarbonyl)-1,2,3,4-tetrahydro-6-isoquinolinecarboxamide,
2-butanoyl-N-(5-{[(2-chlorophenyl)oxy]methyl}-1,3,4-thiadiazol-2-yl)-1,2,3,4-tetrahydro-6-isoquinolinecarboxamide,
N-(5-{[(2-chlorophenyl)oxy]methyl}-1,3,4-thiadiazol-2-yl)-2-propyl-1,2,3,4-tetrahydro-6-isoquinolinecarboxamide,
N-(5-{[(2-chlorophenyl)oxy]methyl}-1,3,4-thiadiazol-2-yl)-2-[5-(hydroxymethyl)-1,3-thiazol-2-yl]-1,2,3,4-tetrahydro-6-isoquinolinecarboxamide, or
5-[(3,4-dichlorophenyl)methyl]-N-(1,2,3,4-tetrahydro-6-isoquinolinyl)-1,3,4-thiadiazole-2-carboxamide hydrochloride.

The compounds of the invention have been found to inhibit SCD activity and may therefore be useful in regulating lipid levels, e.g. plasma lipid levels. Diseases or conditions caused by or associated with an abnormal plasma lipid profile and for the treatment of which the compounds of the invention may be useful include include dyslipidemia, hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia and metabolic syndrome. Other cardiovascular diseases for which the compounds of the present invention may be useful include peripheral vascular disease, reperfusion injury, angioplastic restenosis, hypertension, vascular complications of diabetes and thrombosis. Other diseases or conditions include hepatic steatosis, non-alcoholic steatohepatitis (NASH) and other diseases related to accumulation of lipids in the liver.

The compounds of the invention may also be useful in the treatment of skin disorders e.g. eczema, acne, psoriasis, keloid scar formation or prevention, and diseases related to production or secretions from mucous membranes.

The compounds of the invention may also be useful in the treatment of cancer, neoplasia, malignancy, metastases, tumours (benign or malignant), carcinogenesis, hepatomas and the like.

The compounds of the invention may also be useful in the treatment of mild cognitive impairment (MCI), Alzheimer's disease (AD), cerebral amyloid angiopathy (CAA) or dementia associated with Down Syndrome (DS) and other neurodegenerative diseases characterized by the formation or accumulation of amyloid plaques comprising Aβ42.

Within the context of the present invention, the terms describing the indications used herein are classified in the Merck Manual of Diagnosis and Therapy, 17^{th} Edition and/or the International Classification of Diseases 10^{th} Edition (ICD-10). The various subtypes of the disorders mentioned herein are contemplated as part of the present invention.

In one aspect, the invention provides a compound of formula (I) or a pharmaceutically acceptable salt thereof for use in medical therapy.

In one aspect, the invention provides the use of a compound of formula (I) or a pharmaceutically acceptable salt thereof for the manufacture of a medicament for treating and/or preventing a disease or a condition susceptible to amelioration by an SCD inhibitor.

In another aspect, the invention provides the use of a compound of formula (I) or a pharmaceutically acceptable salt thereof for the manufacture of a medicament for treating and/or preventing acne, cancer, dyslipidemia, hypertriglyceridemia, atherosclerosis, obesity, Type II diabetes, insulin resistance, hyperinsulinaemia, hepatic steatosis and/or non-alcoholic steatohepatitis (NASH).

In another aspect, the invention provides the use of a compound of formula (I) or a pharmaceutically acceptable salt thereof for the manufacture of a medicament for treating and/or preventing acne, cancer, dyslipidemia, atherosclerosis, insulin resistance, hyperinsulinaemia, Type II diabetes and/or hepatic steatosis.

In another aspect, the invention provides the use of a compound of formula (I) or a pharmaceutically acceptable salt thereof for the manufacture of a medicament for treating and/or preventing acne.

In one aspect, the invention provides a compound of formula (I) or a pharmaceutically acceptable salt thereof for use in treating and/or preventing a disease or a condition susceptible to amelioration by an SCD inhibitor in a mammal, including human.

In another aspect, the invention provides a compound of formula (I) or a pharmaceutically acceptable salt thereof for use in treating and/or preventing acne, cancer, dyslipidemia, hypertriglyceridemia, atherosclerosis, obesity, Type II diabetes, insulin resistance, hyperinsulinaemia, hepatic steatosis and/or non-alcoholic steatohepatitis (NASH).

In another aspect, the invention provides a compound of formula (I) or a pharmaceutically acceptable salt thereof for use in treating and/or preventing acne, cancer, dyslipidemia, atherosclerosis, insulin resistance, hyperinsulinaemia, Type II diabetes and/or hepatic steatosis.

In another aspect, the invention provides a compound of formula (I) or a pharmaceutically acceptable salt thereof for use in treating and/or preventing acne.

In one aspect, the invention provides a method for treating and/or preventing a disease or a condition susceptible to amelioration by an SCD inhibitor, which method comprises administering to a subject, for example a mammal, including human, a therapeutically effective amount of a compound of formula (I) or a pharmaceutically acceptable salt thereof.

In another aspect, the invention provides a method for treating and/or preventing a acne, cancer, dyslipidemia, hypertriglyceridemia, atherosclerosis, obesity, Type II diabetes, insulin resistance, hyperinsulinaemia, hepatic steatosis and/or non-alcoholic steatohepatitis (NASH), which method comprises administering to a subject, for example a mammal, including human, a therapeutically effective amount of a compound of formula (I) or a pharmaceutically acceptable salt thereof.

In another aspect, the invention provides a method for treating and/or preventing acne, cancer, dyslipidemia, atherosclerosis, insulin resistance, hyperinsulinaemia, Type II diabetes and/or hepatic steatosis, which method comprises administering to a subject, for example a mammal, including human, a therapeutically effective amount of a compound of formula (I) or a pharmaceutically acceptable salt thereof.

In another aspect, the invention provides a method for treating and/or preventing acne, which method comprises administering to a subject, for example a mammal, including human, a therapeutically effective amount of a compound of formula (I) or a pharmaceutically acceptable salt thereof.

It will be appreciated that reference to “treatment” and “therapy” includes acute treatment or prophylaxis as well as the alleviation of established symptoms.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Processes for the preparation of the compounds of formula (I) form further aspects of the invention. R¹, R², R³, R⁴, R⁵, R⁶, R⁷, X and Y are as defined above unless otherwise specified. Throughout the specification, general formulae are designated by Roman numerals (I), (II), (III), (IV) etc.

In certain instances final compounds of formula (I) can be converted into other compounds of formula (I) by techniques known to those in the art, for example, carboxylic acid substituents can be converted to esters or amides by routine techniques.

In a general process, compounds of formula (I), wherein X represents —NHCO—, Y represents —OCH₂—, —CH₂—, —CH₂CH₂—, —OCH₂CH₂— or —NR⁷CH₂— (wherein R⁷ represents H or —CH₃) and R² represents H (formula (Ia)) may be prepared according to reaction scheme 1 by reacting compounds of formula (III) and compounds of formula (IV), wherein P¹ represents a suitable nitrogen protecting groups such as Boc, to form a compound of formula (II). The reaction is suitably carried out in the presence of a coupling reagent such as HATU, EDCI and/or HOBt, in a suitable solvent such as DCM (suitably at room temperature to reflux) or DMF (suitably at room temperature), and is followed by deprotection of compound of formula (II) under acidic conditions such as hydrochloric acid in a suitable solvent such as ethyl acetate.

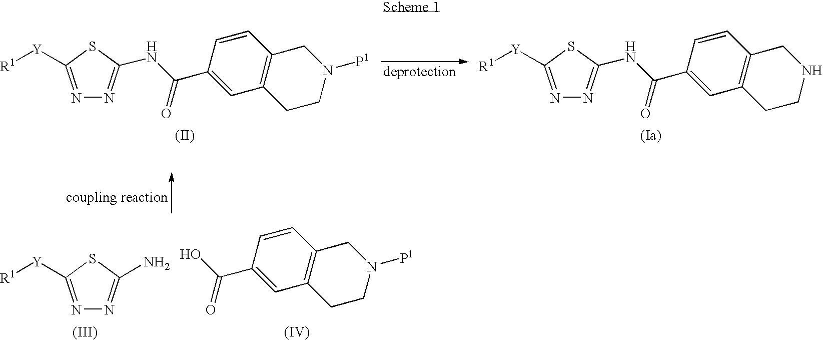

Accordingly, in one aspect the invention provides a process for the preparation of compounds of the formula (Ia) by reacting compounds of formula (III), wherein R¹ and Y are defined above, with compounds of formula (IV), wherein P¹ is defined above, in the presence of a coupling agent, followed by deprotection of compounds of formula (II).

Compounds of formula (I), wherein X represents NHCO—, Y represents —OCH₂—, —CH₂—, —CH₂CH₂— or —OCH₂CH₂— or —NR⁷CH₂— (wherein R⁷ represents H or —CH₃) and R² represents —C₁₋₆alkyl (formula (Ib)) may be prepared according to reaction scheme 2 by reacting compounds of formula (III) and compounds of formula (IVa) in the presence of a coupling reagent such as HATU, EDCI and/or HOBt, in a suitable solvent such as DCM (suitably at room temperature to reflux).

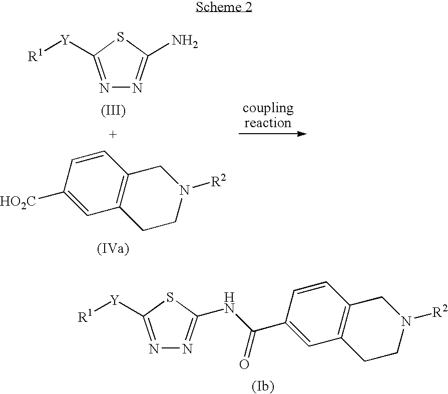

Accordingly, in one aspect the invention provides a process for the preparation of compounds of the formula (Ib) by reacting compounds of formula (III), wherein R¹ and Y are defined above, with compounds of formula (IVa), wherein R² is defined above, in the presence of a coupling agent.

Compounds of formula (I), wherein X represents NHCO—, Y represents —OCH₂—, —CH₂—, —CH₂CH₂— or —OCH₂CH₂— and R² represents —C₁₋₆alkyl (formula Ib) may also be prepared according to reaction scheme 3 by reacting compounds of formula (Ia) with a compound of formula R—CHO wherein R represents —C₁₋₅alkyl (in order to form an R² group which is —CH₂—R) in the presence of reductive agent such as Triacetoxy sodium borohydride, in a suitable solvent such as dichloromethane (suitably at room temperature).

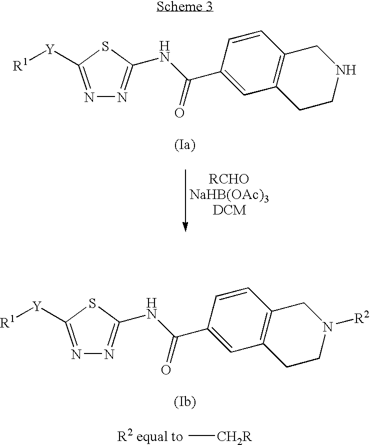

Accordingly, in one aspect the invention provides a process for the preparation of compounds of the formula (Ib) by reacting a compound of formula (Ia), wherein R¹ and Y are defined above, with a compound of formula R—CHO, wherein R is defined above, in the presence of a reductive agent.

Compounds of formula (I), wherein X represents NHCO—, Y represents —OCH₂—, —CH₂—, —CH₂CH₂— or —OCH₂CH₂— and R² represents —C(═O)—C₁₋₆alkyl, —C(═O)—C₃₋₆cycloalkyl or —C(═O)—C₆₋₁₀aryl (formula Ic) may be prepared according to reaction scheme 4 by reacting compounds of formula (Ia) with a compound of formula R²—Cl in the presence of a base such as pyridine, in a suitable solvent such as THF (suitably at room temperature to reflux) or in the presence of a base such as triethylamine in dichloromethane as solvent at room temperature.

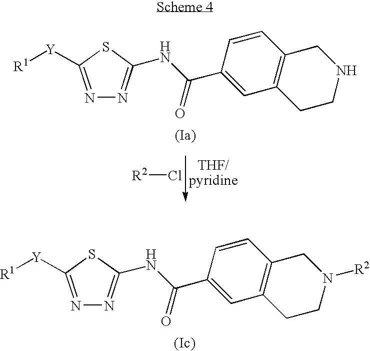

Accordingly, in one aspect the invention provides a process for the preparation of compounds of the formula (Ic) by reacting a compound of formula (Ia), wherein R¹ and Y are defined above, with a compound of formula R²—Cl, wherein R² is defined above, in the presence of a base.

Compounds of formula (I), wherein X represents NHCO—, Y represents —OCH₂—, —CH₂—, —CH₂CH₂— or —OCH₂CH₂— and R² represents —C(═O)—C₁₋₆alkyl, —C(═O)—C₃₋₆cycloalkyl or —C(═O)—C₆₋₁₀aryl (formula Ic) may also be prepared according to reaction scheme 5 by reacting compounds of formula (Ia) with a compound of formula R²—OH in the presence of a coupling reagent such as HATU, EDCI and/or HOBt, in a suitable solvent such as DMF (suitably at room temperature).

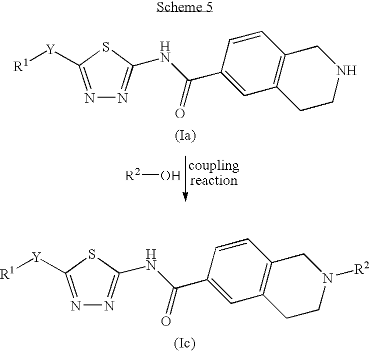

Accordingly, in one aspect the invention provides a process for the preparation of compounds of the formula (Ic) by reacting a compound of formula (Ia), wherein R¹ and Y are defined above, with a compound of formula R²—OH, wherein R² is defined above, in the presence of a coupling reagent.

Compounds of formula (I), wherein X represents N(CH₃)CO—, Y represents —OCH₂—, —CH₂—, —CH₂CH₂— or —OCH₂CH₂— and R² represents H (formula (Id)) may be prepared according to reaction scheme 6 by reacting compounds of formula (II), wherein P¹ represents a suitable nitrogen protecting group such as Boc, with a halogenated methane compound (such as iodomethane) with a base such as sodium hydride in a suitable solvent such as THF (suitably at room temperature) and followed by deprotection of compound of formula (IIa) under acidic conditions such as hydrochloric acid in a suitable solvent such as ethyl acetate.

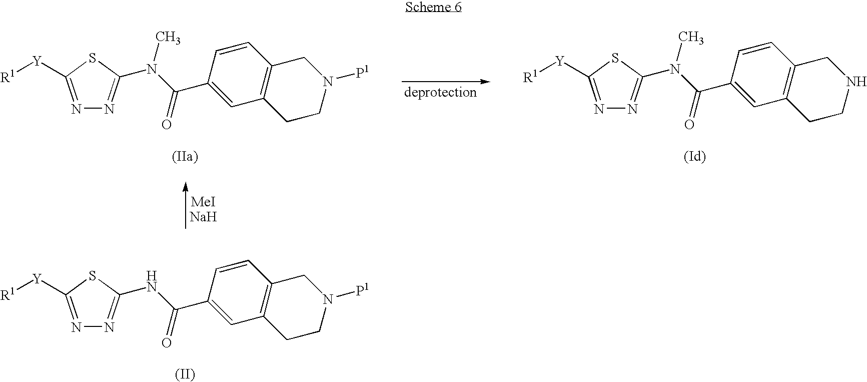

Accordingly, in one aspect the invention provides a process for the preparation of compounds of the formula (Id) by reacting compounds of formula (II), wherein R¹, Y and P¹ are defined above, with a halogenated methane compound, followed by deprotection of compounds of the formula (IIa).

Compounds of formula (I), wherein X represents N(CH₃)CO— and R² is other than H, may be synthesised by methods known to one skilled in the art using compounds of the formula (Id) and the processes described in schemes 3, 4, 5 and 7.

Compounds of formula (I), wherein X represents NHCO—, Y represents —OCH₂—, —CH₂—, —CH₂CH₂— or —OCH₂CH₂— and R² represents -thiazoleCH₂OH (formula Ie) may be prepared according to reaction scheme 7 by reacting compounds of formula (Ia) with (2-bromo-1,3-thiazol-5-yl)methanol in the presence of a base such as DBU, in a suitable solvent such as THF (suitably at room temperature to 60° C.).

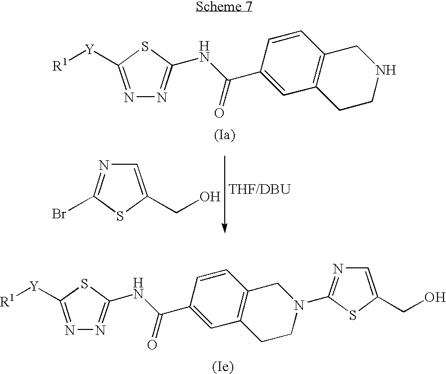

Accordingly, in one aspect the invention provides a process for the preparation of compounds of the formula (Ie) by reacting compounds of formula (Ia), wherein R¹ and

Y are defined above, with (2-bromo-1,3-thiazol-5-yl)methanol in the presence of a base.

Compounds of formula (III) wherein Y represents —OCH₂—, —CH₂—, —CH₂CH₂—, —OCH₂CH₂— or —NR⁷CH₂— (wherein R⁷ represents H or —CH₃) may be prepared according to reaction scheme 8 by reacting compounds of formula (VIII) in the presence of methane sulphonic acid in a suitable solvent such as toluene suitably at reflux or in presence of phosphorous tribromide (suitably at room temperature to 60° C.).

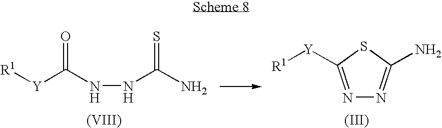

Accordingly, in one aspect the invention provides a process for the preparation of compounds of the formula (III) by reacting compounds of formula (VIII), wherein R¹ and Y are defined above, in the presence of methane sulphonic acid in a suitable solvent.

Compounds of formula (III) wherein Y represents —OCH₂—, —CH₂—, —CH₂CH₂— or —OCH₂CH₂— may also be prepared according to reaction scheme 9 by reacting compounds of formula (IX) in the presence of hydrazinecarbothioamide in a suitable solvent such as polyphosphoric acid (suitably at room temperature to 110° C.).

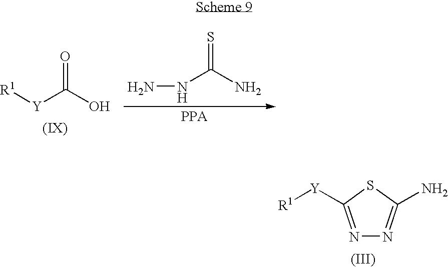

Compounds of formula (III) wherein Y represents —OCH₂—, —CH₂—, —CH₂CH₂— or —OCH₂CH₂— may also be prepared according to reaction scheme 10 by reacting compounds of formula (XIV) in the presence of hydrazinecarbothioamide in a suitable solvent such as trifluoroacetic acid (suitably at reflux). Compounds of formula (XIV) when Y is —OCH₂— may be prepared according to reaction scheme 8 by reacting compound of formula (XII) with a reagent such as 2-chloroacetonitrile in the presence of a base such as potassium carbonate in a suitable solvent such as acetone

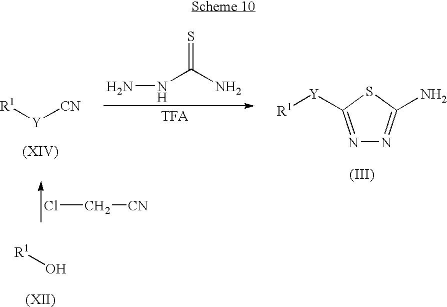

Compounds of formula (VIII) wherein Y represents —OCH₂—, —CH₂—, —CH₂CH₂—, —OCH₂CH₂— or —NR⁷CH₂— (wherein R⁷ represents H or —CH₃) may be prepared according to reaction scheme 11 by reacting compounds of formula (IX) with hydrazinecarbothioamide in the presence of a coupling reagent such as HATU, EDCI and/or HOBt, in a suitable solvent such as DMF (suitably at room temperature to 80° C.). Compounds of formula (VIII) may also be prepared according to reaction scheme 11 by reacting compounds of formula (X) with hydrazinecarbothioamide with a base such as pyridine in a suitable solvent such as DMF (suitably at room temperature to reflux). Compounds of formula (X) may be prepared by reacting compounds of formula (IX) with a chlorinating agent such as oxalyl chloride or thionyl chloride in a suitable solvent such as dichloromethane.

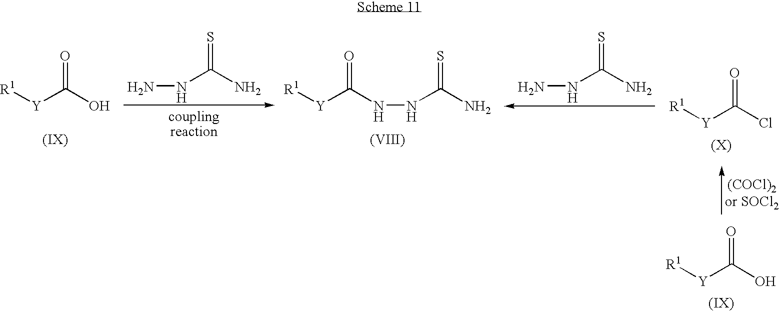

Compounds of formula (XI), wherein Y represents —OCH₂—, may be prepared according to reaction scheme 12 by reacting compounds of formula (XII) with a reagent such as ethyl bromoacetate or ethyl chloroacetate in the presence of a base such as potassium carbonate in a suitable solvent such as acetone, followed by saponification of compound of formula (XIII) with a base such as sodium hydroxide or potassium hydroxide in a suitable solvent such as ethanol or methanol (suitably at room temperature to reflux).

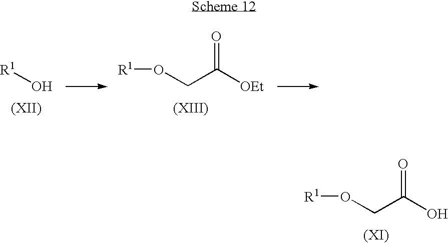

Compounds of formula (IX) wherein Y represents —OCH₂—, —CH₂—, —CH₂CH₂— or —OCH₂CH₂— may be prepared according to reaction scheme 13 by reacting compounds of formula (XIV) with a reagent such as sodium hydroxide in a suitable solvent such as water (suitably at room temperature to reflux).

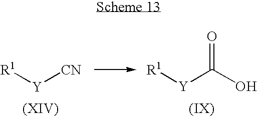

Compounds of formula (I), wherein X represents CONH—, Y represents —OCH₂—, —CH₂CH₂— or —OCH₂CH₂— and R² represents hydrogen (formula (If)) may be prepared according to reaction scheme 14 by deprotection of compounds of formula (V), wherein P¹ represents a suitable nitrogen protecting group such as Boc, under acidic conditions such as hydrochloric acid or trifluoracetic acid.

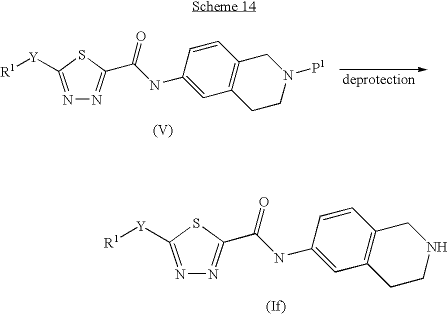

Accordingly, in one aspect the invention provides a process for the preparation of compounds of the formula (If), wherein R¹ and Y are defined above, by deprotecting compounds of the formula V, wherein P¹ is defined above, under acidic conditions.

Compounds of formula (V), wherein X represents —CONH— and Y represents —OCH₂—, may be prepared according to reaction scheme 15 by reacting compounds of formula (XX), wherein P¹ represents a suitable nitrogen protecting groups such as Boc, with a compound of formula (XII) in the presence of a base such as potassium carbonate in a suitable solvent such as acetone (suitably at room temperature to reflux).

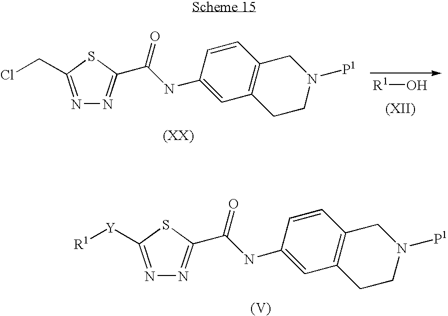

Compounds of formula (I), wherein X represents —CONH—, Y represents —CH₂— and R² represents hydrogen (formula (If)), may be prepared according to reaction scheme 16 by reacting compounds of formula (XXII), wherein P¹ represents a suitable nitrogen protecting group such as Boc, and compounds of formula (XXIII) to form a compound of formula (XXI). The reaction is suitably carried out in the presence of a coupling reagent such as HATU, EDCI and/or HOBt, in a suitable solvent such as DCM (suitably at room temperature to reflux) or DMF (suitably at room temperature), and is followed by reaction of compound of formula (XXI) in presence of phosphorous tribromide in a suitable solvent such as dichloromethane (suitably at room temperature to reflux).

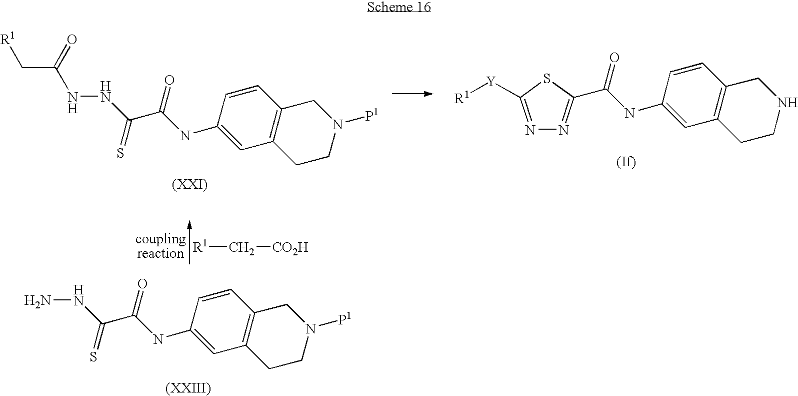

Accordingly, in one aspect the invention provides a process for the preparation of compounds of the formula (If), wherein R¹ and Y are defined above, by reacting compounds of formula (XXII) and compounds of formula (XXIII), followed by reaction of compound of formula (XXI) in presence of phosphorous tribromide.

Compounds of formula (I), wherein X represents —CONH— and R² is other than H, may be synthesised by methods known to one skilled in the art using compounds of the formula (If) and the processes as described in schemes 3, 4, 5 and 7.

Compounds of formula (XX) may be prepared according to reaction scheme 17 by reacting compounds of formula (XXII) with a reagent such as chloroacetyl chloride in a suitable solvent such as DMF (suitably at room temperature).

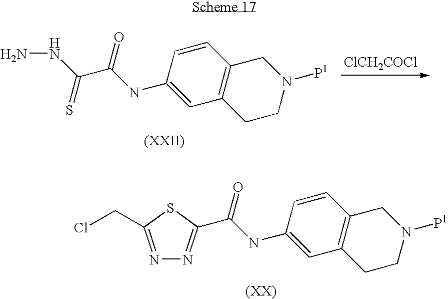

Compounds of formula (XXII) may be prepared according to reaction scheme 18 by reacting compounds of formula (XXV), wherein P¹ represents a suitable nitrogen protecting group such as Boc, with a reagent such as sulphur and morpholine in a suitable solvent such as DMF (suitably at room temperature). The reaction is followed by reaction of compounds of formula (XXIV) with a reagent such as hydrazine hydrate in a suitable solvent such as DMF (suitably at room temperature).

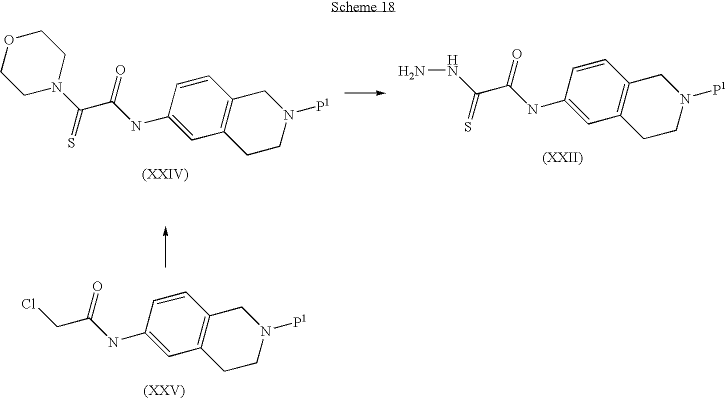

Compounds of formula (XXV) may be prepared according to reaction scheme 19 by reacting compounds of formula (VII), wherein P¹ represents a suitable nitrogen protecting group such as Boc, with a reagent such as chloroacetyl chloride in a suitable solvent such as THF (suitably at room temperature).

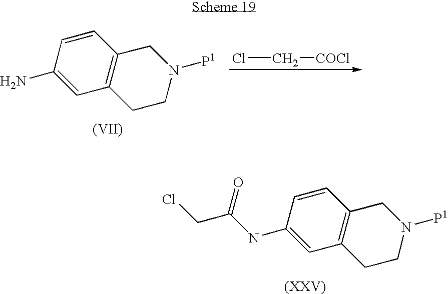

Compounds of the formula (IV), (VI) (VII), (XII), (XIV) and (XXIII) are commercially available compounds or may be prepared by methods known in the literature or processes known to those skilled in the art.

Further details for the preparation of compounds of formula (I) are found in the examples section hereinafter.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel syntheses using either solution phase or solid phase chemistry, by procedures known to those skilled in the art. Thus according to a further aspect there is provided a compound library comprising at least 2 compounds of the invention.

Those skilled in the art will appreciate that in the preparation of compounds of formula (I) and/or salts thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl).

Various intermediate compounds used in the above-mentioned process, including but not limited to certain compounds of formulae (II), (V), constitute a further aspect of the present invention.

The compounds of formula (I) or pharmaceutically acceptable salt(s) thereof may also be used in combination with other therapeutic agents. The invention thus provides, in a further aspect, a combination comprising a compound of formula (I) or pharmaceutically acceptable salt thereof together with one or more further therapeutic agent(s).

Compounds of the invention may be administered in combination with other therapeutic agents. Preferred therapeutic agents are selected from the list: an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA: cholesterol acyltransferase (ACAT inhibitor), a cannabinoid 1 antagonist a bile acid sequestrant a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor.

When the compound of formula (I) or pharmaceutically acceptable salt thereof is used in combination with a second therapeutic agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention. The individual components of such combinations may be administered either sequentially or simultaneously in separate or combined pharmaceutical formulations by any convenient route.

When administration is sequential, either the SCD inhibitor or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The invention also includes a pharmaceutical composition comprising one or more compounds of formula (I) or pharmaceutically acceptable salt(s) in combination with one or more excipients.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be in the form of tablets, capsules, powders, granules, lozenges, creams or liquid preparations, such as oral or sterile parenteral solutions or suspensions.

The topical formulations of the present invention may be presented as, for instance, dispersions, lotions, creams, gels, pastes, powders, aerosol sprays, syrups or ointments on sponges or cotton applicators, and solutions or suspensions in an aqueous liquid, non-aqueous liquid, oil-in-water emulsion, or water-in-oil liquid emulsion.

Creams, lotions, or ointments, may be prepared as rinse-off or leave-on products, as well as two stage treatment products for use with other skin cleansing or managing compositions. The compositions can be administered as a rinse-off product in a higher concentration form, such as a gel, and then a leave-on product in a lower concentration to avoid irritation of the skin. Each of these forms is well understood by those of ordinary skill in the art, such that dosages may be easily prepared to incorporate the pharmaceutical composition of the invention.

Ointments are hydrocarbon-based semisolid formulations containing dissolved or suspended drugs. Creams and lotions are semi-solid emulsion systems and the term is applied both to water/oil or oil/water. Gel formulations are semi-solid systems in which a liquid phase is trapped in a polymeric matrix.

By way of non-limiting example, the ointments may contain one or more hydrophobic carriers selected from, for example, white soft paraffin or other mineral waxes, liquid paraffin, non-mineral waxes, long chain alcohols, long chain acids and silicones. The ointment may contain in addition to the hydrophobic carriers some hydrophillic carriers selected from, for example, propylene glycol and polyethylene glycol in combination with an appropriate surfactant/co-surfactant system. The carrier compositions of the creams or lotions are typically based on water, white soft paraffin and an appropriate surfactant/co-surfactant system, in combination with other carriers/components selected from, for example, propylene glycol, butylene glycol glycerinemonostearate, PEG-glycerinemonostearate, esters such as C₁₂₋₁₅ alkyl benzoate, liquid paraffin, non-mineral waxes, long chain alcohols, long chain acids silicones, non-silicone polymers. The gels may by way of example be formulated using isopropyl alcohol or ethyl alcohol, propylene glycol and water with a gelling agent such as hydroxyethyl cellulose, suitably in combination with minor components, for example one or more of butylene glycol and a wetting agent such as a poloxamer.

An ointment, cream, lotion, gel, and the like, can further comprise a moisturizing agent. The moisturizing agent can be a hydrophobic moisturizing agent such as ceramide, borage oil, tocopherol, tocopherol linoleate, dimethicone or a mixture thereof or a hydrophilic moisturizing agent such as glycerine, hyaluronic acid, sodium peroxylinecarbolic acid, wheat protein, hair keratin amino acids, or a mixture thereof.

The compositions according to the invention may also comprise conventional additives and adjuvants for dermatological applications, such as preservatives, acids or bases used as pH buffer excipients and antioxidants.

The present invention encompasses administration via a transdermal patch or other forms of transdermal administration. Suitable formulations for transdermal administration are known in the art, and may be employed in the methods of the present invention. For example, suitable transdermal patch formulations for the administration of a pharmaceutical compound are described in, for example, U.S. Pat. No. 4,460,372 to Campbell et al., U.S. Pat. No. 4,573,996 to Kwiatek et al., U.S. Pat. No. 4,624,665 to Nuwayser, U.S. Pat. No. 4,722,941 to Eckert et al., and U.S. Pat. No. 5,223,261 to Nelson et al.

One suitable type of transdermal patch for use in the methods of the present invention encompasses a suitable transdermal patch includes a backing layer which is non-permeable, a permeable surface layer, an adhesive layer substantially continuously coating the permeable surface layer, and a reservoir located or sandwiched between the backing layer and the permeable surface layer such that the backing layer extends around the sides of the reservoir and is joined to the permeable surface layer at the edges of the permeable surface layer. The reservoir contains a compound of formula (I) or pharmaceutically acceptable salt thereof, alone or in combination, and is in fluid contact with the permeable surface layer. The transdermal patch is adhered to the skin by the adhesive layer on the permeable surface layer, such that the permeable surface layer is in substantially continuous contact with the skin when the transdermal patch is adhered to the skin. While the transdermal patch is adhered to the skin of the subject, the compound of formula (I) or pharmaceutically acceptable salt thereof contained in the reservoir of the transdermal patch is transferred via the permeable surface layer, from the reservoir, through the adhesive layer, and to the skin of the patient. The transdermal patch may optionally also include one or more penetration-enhancing agents in the reservoir that enhance the penetration of the compound of formula (I) or pharmaceutically acceptable salt thereof through the skin.

Examples of suitable materials which may comprise the backing layer are well known in the art of transdermal patch delivery, and any conventional backing layer material may be employed in the transdermal patch of the instant invention.

Suitable penetration-enhancing agents are well known in the art as well. Examples of conventional penetration-enhancing agents include alkanols such as ethanol, hexanol, cyclohexanol, and the like, hydrocarbons such as hexane, cyclohexaue, isopropylbenzene; aldehydes and ketones such as cyclohexanone, acetamide, N,N-di(lower alkyl)acetamides such as N,N-diethylacetamide, N,N-dimethyl acetamide, N-(2-hydroxyethyl)acetamide, esters such as N,N-di-lower alkyl sulfoxides; essential oils such as propylene glycol, glycerine, glycerol monolaurate, isopropyl myristate, and ethyl oleate, salicylates, and mixtures of any of the above.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Preparations for oral administration may be suitably formulated to give controlled/extended release of the active compound.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active ingredient, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

The invention also extends to novel intermediates disclosed herein, used in the preparation of compounds of formula (I)

### DEFINITIONS

AcOEt ethyl acetate
Boc tertbutyloxy carbonyl
CCl₄ carbon tetrachloride
DIPEA diisopropylethylamine
DCM dichloromethane
DMF dimethylformamide
Et₃N triethylamine
EtOAc ethyl acetate
EtOH ethanol
Fmoc 9-Fluorenylmethoxycarbonyl
HATU O-(7-Azabenzotriazol-1-yl)-1,1,3,3-tetramethyluronium hexafluorophosphate
HCl hydrochloric acid
HOBt 1-hydroxybenzotriazole
m-CPBA meta chloroperbenzoic acid
MeCN acetonitrile
Me methyl
MeOH methanol
NaBH₃CN sodium cyanoborohydride
NaHB(OAc)₃ triacetoxy sodium borohydride
NaOH sodium hydroxide
Net₃ triethylamine
NH₂NH₂ hydrazine
PPA polyphosphoric acid
Pd(PPh₃)₄ Palladium tetrakis

Regardless of how the preparation of compounds are represented in the present specification no inference can be drawn that particular batches (or mixtures of two or more batches) of intermediates were used in the next stage of the preparation. The examples and intermediates are intended to illustrate the synthetic routes suitable for preparation of the same, to assist the skilled persons understanding of the present invention.

Where reference is made to the use of a “similar” procedure, as will be appreciated by those skilled in the art, such a procedure may involve minor variation, for example reaction temperature, reagent/solvent amount, reaction time, work-up conditions or chromatographic purification conditions.

### Analytical Methods LC-MS

Analytical HPLC was conducted on a X-terra MS C18 column (2.5 μm 3×30 mm id) eluting with 0.01M ammonium acetate in water (solvent A) and 100% acetonitrile using the following elution gradient: 0 to 4 minutes, 5 to 100% B; 4 to 5 minutes, 100% B at a flow-rate of 1.1 mL/min with a temperature of 40° C.

The mass spectra (MS) were recorded on a micromass ZQ-LC mass spectrometer using electrospray positive ionisation [ES+ve to give MH⁺ molecular ion] or electrospray negative ionisation [ES-ve to give (M−H)⁻ molecular ion] modes.

### Analytical Methods LC-HRMS

Analytical HPLC was conducted on an Uptisphere-hsc column (3 μm 30×3 mm id) eluting with 0.01M ammonium acetate in water (solvent A) and 100% acetonitrile (solvent B) using the following elution gradient: 0 to 0.5 minutes, 5% B; 0.5 to 3.5 minutes, 5 to 100% B; 3.5 to 4 minutes, 100% B; 4 to 4.5 minutes, 100 to 5% B; 4.5 to 5.5 minutes, 5% B at a flow-rate of 1.3 mL/min with a temperature of 40° C.

The mass spectra (MS) were recorded on a micromass LCT, mass spectrometer using electrospray positive ionisation [ES+ve to give MH⁺ molecular ion] or electrospray negative ionisation [ES−ve to give (M−H)⁻ molecular ion] modes.

### Analytical Method GC-MS

Analytical GC was conducted on a DB-1 ms column (Agilent Technologies), 0.1 μm 10 m×0.1 mm id) eluting with an Helium flow of 0.5 ml/min and pressure at 3.4 bar and with a gradient temperature: 0 to 0.35 min, 100° C.; 0.35 min to 6 min, 100° C. to 250° C. (ramp of 80° C./min).

The mass spectra (MS) were recorded on a Agilent Technologies G5973 mass spectrometer using electronic impact ionisation.

The following non-limiting examples illustrate the present invention.

### Intermediate 1: 5-Chloro-2-[(2-methylpropyl)oxy]phenol

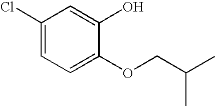

5-Chloro-2-[(2-methylpropyl)oxy]benzaldehyde (10 g, 47.17 mmol) was dissolved in dichloromethane (80 ml). The solution was cooled to 65° C. m-CPBA 85% (8.85 g, 51.45 mmol) was added slowly. The mixture was stirred at room temperature overnight. The solid was filtered and the filtrate was concentrated. The residue was diluted with methanol (80 ml). The solution was cooled below 20° C. and a solution of NaOH 20% (43 ml, 0.215 mol) was added. The mixture was stirred for 30 min and was acidified with concentrated HCl. The mixture was cooled to 5° C. The solid was filtered, washed with cold water. The solid was dissolved with ethyl acetate, washed with brine, dried over Na₂SO₄ and the organic phase was concentrated. The residue was purified by flash column chromatography eluting with pentane/ethyl acetate 10:1 to give the title compound (9 g, 96%).

¹H NMR (300 MHz, CDCl₃, ppm) δ: 6.9 (s, 1H), 6.8 (m, 2H), 3.80 (d, 2H), 2.10 (m, 1H), 1.00 (d, 6H).

The following Intermediates were prepared using the generic reaction scheme (Scheme 12)

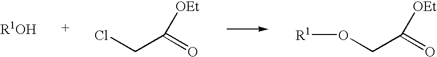

### Intermediate 2: Ethyl [(4-chlorophenyl)oxy]acetate

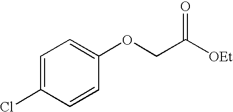

To a solution of 4-chlorophenol (25.6 g, 0.2 mol.) in DMF were added potassium carbonate (41.4 g, 0.2 mol.) and then drop-wise, ethyl chloroacetate (21.2 ml, 0.2 mol.). The solution was heated at 70° C. overnight. After filtration, the filtrate was poured into water and extracted with ethyl acetate. The organic layer was washed with water then brine, dried on sodium sulphate and evaporated to dryness to give the title compound as a dark oil (30 g, 70%).

LC/MS: m/z 215 (M+H)⁺, Rt: 4.66 min.

The following Intermediates were similarly prepared by a method analogous to that described for Intermediate 2.

TABLE 1

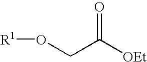

| Intermediate No. | R¹ | From: | Physical data |
|---|---|---|---|
| 3 Ethyl [(2-fluorophenyl)oxy] acetate | 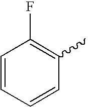 | Commercially available | LC/MS: m/z 199 (M + H)+ Rt: 3.34 min |
| 4 Ethyl [(3-fluorophenyl)oxy] acetate | 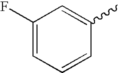 | Commercially available | LC/MS: m/z 199 (M + H)+ Rt: 3.39 min |
| 5 Ethyl [(4-fluorophenyl)oxy] acetate | 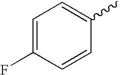 | Commercially available | LC/MS: m/z 199 (M + H)+ Rt: 3.26 min |
| 6 Ethyl [(2,5-dichlorophenyl) oxy]acetate | 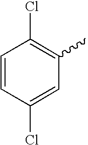 | Commercially available | ¹H NMR (300 MHz, CDCl₃, ppm) δ: 7.33 (d, 1 H, J = 8.54 Hz), 6.96 (dd, 1 H, J = 2.25 Hz, 8.41 Hz), 8.85 (d, 1 H, J = 2.08 Hz), 4.71 (s, 2 H), 4.31 (q, 2 H, J = 7.15 Hz), 1.33 (t, 3 H, J = 7.15 Hz). |
| 7 Ethyl [(2-chloro-4- fluorophenyl)oxy]acetate | 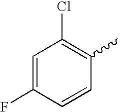 | Commercially available | ¹H NMR (300 MHz, CDCl₃, ppm) δ: 7.08 (dd, 1 H, J = 3.04 Hz, 7.95 Hz), 6.81 (m, 2 H), 4.59 (s, 2 H), 4.19 (q, 2 H, J = 7.29 Hz), 1.22 (t, 3 H, J = 7.29 Hz). |
| 8 Ethyl [(2-chloro-5- fluorophenyl)oxy]acetate | 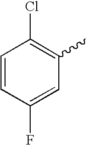 | Commercially available | ¹H NMR (300 MHz, CDCl₃, ppm) δ: 7.35 (dd, 1 H, J = 6.10 Hz, 8.71 Hz), 6.70 (td, 1 H, J = 2.67 Hz, 8.06 Hz), 6.60 (dd, 1 H, J = 2.61 Hz, 10.07 Hz), 4.70 (s, 2 H), 4.30 (q, 2 H, J = 7.14 Hz), 1.32 (t, 3 H, J = 7.33 Hz). |
| 9 Ethyl [(3,4- dichlorophenyl)oxy]acetate | 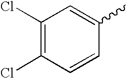 | Commercially available | ¹H NMR (300 MHz, CDCl₃, ppm) δ: 7.4 (d, 1 H), 7.0 (s, 1 H), 6.8 (d, 1 H), 4.6 (s, 2 H), 4.2 (q, 2 H), 1.3 (t, 3 H). |
| 10 Ethyl (5,6,7,8-tetrahydro- 1-naphthalenyloxy)acetate | 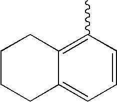 | Commercially available | ¹H NMR (300 MHz, CDCl₃, ppm) δ: 7.05 (t, 1 H, J = 8.07 Hz), 6.76 (d, 1 H, 7.83 Hz), 6.54 (d, 1 H, J = 8.31 Hz), 4.64 (s, 2 H), 4.29 (q, 2 H, J = 7.12 Hz), 2.78 (m, 4 H), 1.81 (m, 4 H), 1.33 (t, 3 H, J = 7.12 Hz). |
| 11 Ethyl {[3- (trifluoromethyl)phenyl] oxy}acetate | 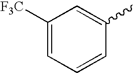 | Commercially available | ¹H NMR (300 MHz, CDCl₃, ppm) δ: 7.40 (t, 1 H), 7.26 (dd, 1 H), 7.14 (s, 1 H), 7.1 (dd, 1 H), 4.65 (s, 2 H), 4.27 (q, 2 H), 1.29 (t, 3 H). |
| 12 Ethyl [(3-chlorophenyl)oxy] acetate | 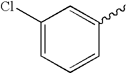 | Commercially available | LC/MS: m/z 215 (M + H)+ Rt: 3.49 min |
| 13 Ethyl {[2- (trifluoromethyl)phenyl] oxy}acetate | 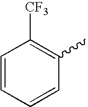 | Commercially available | ¹H NMR (300 MHz, CDCl₃, ppm) δ: 7.6 (dd, 1 H), 7.5 (t, 1 H), 7.06 (t, 1 H), 6.87 (d, 1 H), 4.72 (s, 2 H), 4.26 (q, 2 H), 1.28 (t, 3 H). |
| 14 Ethyl (1-naphthalenyloxy) acetate | 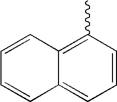 | Commercially available | ¹H NMR (300 MHz, DMSO, ppm) δ: 8.40 (m, 1 H), 7.83 (m, 1 H), 7.53 (m, 3 H), 7.37 (t, 1 H, J = 7.78 Hz), 6.74 (d, 1 H, J = 7.56 Hz), 4.83 (s, 2 H), 4.33 (q, 2 H, J = 7.30 Hz), 1.34 (t, 3 H, J = 7.30 Hz). |
| 15 Ethyl {[4- (methyloxy)phenyl] oxy}acetate | 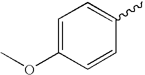 | Commercially available | ¹H NMR (300 MHz, DMSO, ppm) δ: 6.80 (s, 4 H), 4.6 (s, 2 H), 4.3 (q, 2 H), 3.75 (s, 3 H), 1.3 (t, 3 H). |
| 16 Ethyl (2-biphenylyloxy) acetate | 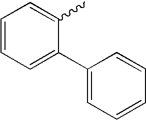 | Commercially available | ¹H NMR (300 MHz, DMSO, ppm) δ: 7.49 (d, 2 H), 7.35 (t, d, 5 H), 7.1 (dd, 2 H), 4.8 (s, 2 H), 4.1 (q, 2 H), 1.2 (t, 3 H). |
| 17 Ethyl ({5-chloro-2-[(2- methylpropyl)oxy]phenyl} oxy)acetate | 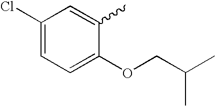 | 5-Chloro-2-[(2- methylpropyl) oxy]phenol (Intermediate 1) | ¹H NMR (300 MHz, DMSO, ppm) δ: 6.95-6.70 (m, 3 H), 4.62 (s, 2 H), 4.22 (q, 2 H), 3.75 (d, 2 H), 2.10 (m, 1 H), 1.25 (t, 3 H), 1.05 (d, 6 H). |
| 18 Ethyl [(2,4-dichlorophenyl) oxy]acetate | 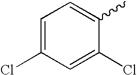 | Commercially available | ¹H NMR (300 MHz, DMSO, ppm) δ: 7.42 (d, 1 H), 7.19 (dd, 1 H), 6.49 (d, 1 H), 4.70 (s, 2 H), 4.27 (q, 2 H), 1.31 (t, 3 H). |
| 19 Ethyl {[2- (methyloxy)phenyl] oxy}acetate | 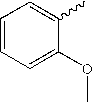 | Commercially available | Not isolated |

The following Intermediates were prepared using the generic reaction scheme (Scheme 12)

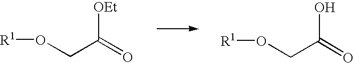

### Intermediate 20: [(4-Chlorophenyl)oxy]acetic acid

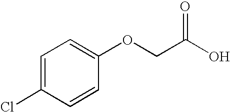

To a solution of ethyl [(4-chlorophenyl)oxy]acetate (Intermediate 2) (60 g, 0.28 mol.) in methyl alcohol was added a solution of potassium hydroxide (28 g, 0.5 mol.) in water. The solution was heated at 70° C. overnight. After concentration under reduced pressure, the mixture was cooled with iced water and concentrated HCl (20 ml, 10 M) was added. The resulting solid material was filtered and dried to give the title compound as a white solid (40 g, 77%).

¹H NMR (300 MHz, CDCl₃, ppm) δ: 7.3 (d, 2H), 6.9 (d, 2H), 4.6 (s, 2H).

The following compounds were similarly prepared by a method analogous to that described for Intermediate 20.

TABLE 2

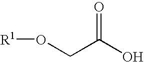

| Intermediate No | R¹ | From Intermediate No. | Physical data |
|---|---|---|---|
| 21 [(2-fluorophenyl)oxy] acetic acid | 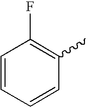 | 3 Ethyl [(2-fluorophenyl) oxy]acetate | LC/MS: m/z 171 (M + H)⁺ Rt: 2.54 min |
| 22 [(3-fluorophenyl)oxy] acetic acid | 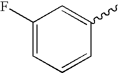 | 4 Ethyl [(3-fluorophenyl) oxy]acetate | LC/MS: m/z 171 (M + H)⁺ Rt: 2.70 min |
| 23 [(4-fluorophenyl)oxy] acetic acid | 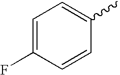 | 5 Ethyl [(4-fluorophenyl) oxy]acetate | ¹H NMR (300 MHz, DMSO, ppm) δ: 7.1 (d, 2 H), 6.94 (d, 2 H), 4.65 (s, 2 H). |
| 24 [(2,5-dichlorophenyl) oxy]acetic acid | 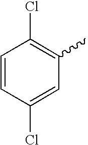 | 6 Ethyl [(2,5- dichlorophenyl)oxy] acetate | ¹H NMR (300 MHz, CDCl3, ppm) δ: 7.46 (d, 1 H, J = 8.39 Hz), 7.16 (d, 1 H, J = 2.42 Hz), 7.04 (dd, 1 H, J = 2.42 Hz, 8.57 Hz), 4.86 (s, 2 H). |
| 25 [(2-chloro-4- fluorophenyl)oxy] acetic acid | 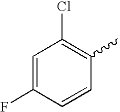 | 7 Ethyl [(2-chloro-4- fluorophenyl)oxy]acetate | ¹H NMR (300 MHz, DMSO, ppm) δ: 7.45 (dd, 1 H, J = 2.99 Hz, 8.35 Hz), 7.16 (td, 1 H, J = 3.07 Hz, 9.12 Hz), 7.06 (dd, 1 H, J = 5.14 Hz, 9.27 Hz), 4.80 (s, 2 H). |
| 26 [(2-chloro-5- fluorophenyl)oxy] acetic acid | 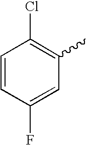 | 8 Ethyl [(2-chloro-5- fluorophenyl)oxy]acetate | ¹H NMR (300 MHz, DMSO, ppm) δ: 7.46 (dd, 1 H, J = 6.29 Hz, 8.76 Hz), 6.98 (dd, 1 H, J = 2.88 Hz, 10.90 Hz), 6.82 (td, 1 H, J = 2.81 Hz, 8.36 Hz), 4.78 (s, 2 H). |
| 27 [(3,4- dichlorophenyl)oxy] acetic acid | 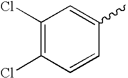 | 9 Ethyl [(3,4- dichlorophenyl) oxy]acetate | ¹H NMR (300 MHz, CDCl3, ppm) δ: 7.4 (d, 1 H), 7.05 (s, 1 H), 6.8 (d, 1 H), 4.6 (s, 2 H). |
| 28 (5,6,7,8-tetrahydro- 1-naphthalenyloxy) acetic acid | 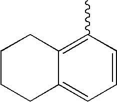 | 10 Ethyl (5,6,7,8- tetrahydro-1- naphthalenyloxy) acetate | ¹H NMR (300 MHz, DMSO, ppm) δ: 7.00 (t, 1 H, J = 8.14 Hz), 6.66 (d, 1 H, J = 7.74 Hz), 6.58 (d, 1 H, J = 8.14 Hz), 4.65 (s, 2 H), 2.69 (m, 2 H), 2.61 (m, 2 H), 1.70 (m, 4 H). |
| 29 {[3-(trifluoromethyl) phenyl]oxy}acetic acid | 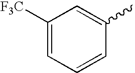 | 11 Ethyl {[3-(trifluoromethyl) phenyl]oxy}acetate | ¹H NMR (300 MHz, DMSO, ppm) δ: 7.5 (t, 1 H), 7.3 (m, 3 H), 4.8 (s, 2 H). |
| 30 [(3-chlorophenyl)oxy] acetic acid | 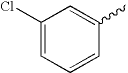 | 12 Ethyl [(3-chlorophenyl) oxy]acetate | Not isolated |
| 31 {[2-(trifluoromethyl) phenyl]oxy}acetic acid | 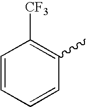 | 13 Ethyl {[2-(trifluoromethyl) phenyl]oxy}acetate | LC/MS: m/z 250 (M + H)⁺ Rt: 3.30 min |
| 32 (1-naphthalenyloxy) acetic acid | 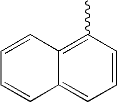 | 14 Ethyl (1- naphthalenyloxy) acetate | ¹H NMR (300 MHz, DMSO, ppm) δ: 8.21 (m, 1 H), 7.89 (m, 1 H), 7.53 (m, 3 H), 7.40 (t, 1 H, J = 7.80 Hz), 6.88 (d, 1 H, J = 7.54 Hz), 4.88 (s, 2 H). |
| 33 {[4- (methyloxy)phenyl] oxy}acetic acid | 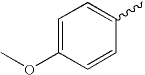 | 15 Ethyl {[4- (methyloxy)phenyl] oxy}acetate | ¹H NMR (300 MHz, DMSO, ppm) δ: 6.9 (s, 4 H), 4.5 (s, 2 H), 3.6 (s, 3 H). |
| 34 (2-biphenylyloxy) acetic acid | 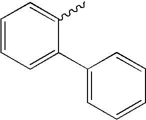 | 16 Ethyl (2-biphenylyloxy) acetate | ¹H NMR (300 MHz, DMSO, ppm) δ: 7.6-6.9 (m, 9 H), 4.3 (s, 2 H). |
| 35 ({5-chloro-2-[(2- methylpropyl)oxy] phenyl}oxy)acetic acid | 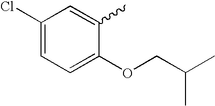 | 17 Ethyl ({5-chloro-2-[(2- methylpropyl)oxy] phenyl}oxy)acetate | ¹H NMR (300 MHz, DMSO, ppm) δ: 7.00-6.80 (m, 3 H), 4.6 (s, 2 H), 3.75 (d, 2 H), 2.00 (m, 1 H), 1.00 (d, 6 H). |
| 36 [(2,4-dichlorophenyl) oxy]acetic acid | 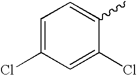 | 18 Ethyl [(2,4- dichlorophenyl) oxy]acetate | ¹H NMR (300 MHz, DMSO, ppm) δ: 7.59 (d, 1 H), 7.35 (dd, 1 H), 7.607 (d, 1 H), 4.84 (s, 2 H). |
| 37 {[2- (methyloxy)phenyl] oxy}acetic acid | 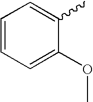 | 19 Ethyl {[2-(methyloxy) phenyl] oxy}acetate | ¹H NMR (300 MHz, DMSO, ppm) δ: 7.1 (m, 4 H), 4.75 (s, 2 H), 3.8 (s, 3 H). |

The following Intermediates were prepared using the generic reaction scheme (Scheme 13)

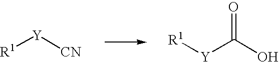

### Intermediate 38: [3-(Trifluoromethyl)phenyl]acetic acid

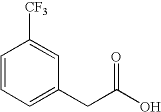

A solution of [3-(trifluoromethyl)phenyl]acetonitrile (5.4 g, 0.03 mmol) and NaOH (6 g, 0.15 mmol) in water was refluxed overnight. After cooling, the pH was adjusted to 2 with dilute HCl. The precipitate was filtered, washed with water and dried to give the title compound as a solid (5.2 g, 87%).

¹H NMR (300 MHz, DMSO, ppm) δ: 7.6 (m, 4H), 3.7 (s, 2H).

The following Intermediates were prepared using the generic reaction scheme (Scheme 11)

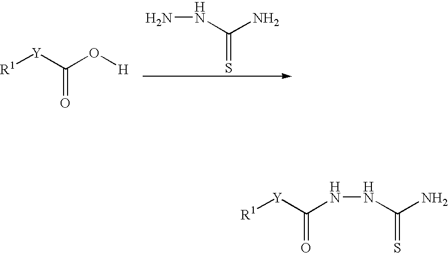

### Intermediate 39: 2-{[(2-Chlorophenyl)oxy]acetyl}hydrazinecarbothioamide

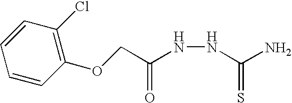

Three coupling reactions were carried out simultaneously on a 10 g scale.

A solution of 2-chlorophenoxyacetic acid (10 g, 54 mmol), HATU (22.4 g, 59 mmol) and NEt₃ (11.1 mL, 80 mmol) in DMF was stirred at room temperature for 1 hour. Hydrazinecarbothioamide (5.9 g, 64 mmol) was added, and the reaction mixture was stirred at room temperature for two days. After evaporation under reduced pressure of the solvent of the three combined mixtures, the residue was diluted with water and the formed precipitate was filtered and dried to give the title compound as a pale yellow powder.

Total yield: 36.6 g, 87%.

LC/MS: m/z 260 (WH)⁺, Rt: 2.09 min.

The following Intermediates were similarly prepared by a method analogous to that described for Intermediate 39.

TABLE 3

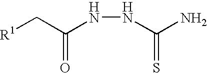

| | | From | |
|---|---|---|---|
| Intermediate No. | R¹ | Intermediate No. | Physical data |
| 40 2-(1H-indol-3-ylacetyl) hydrazinecarbothioamide | 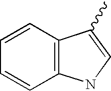 | Commercially available | LC/MS: m/z 249 (M + H)⁺, Rt: 1.49 min. |
| 41 2-{[(2,5- dichlorophenyl)oxy]acetyl} hydrazinecarbothioamide | 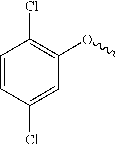 | 24 [(2,5- dichlorophenyl) oxy]acetic acid | LC/MS: m/z 295 (M + H)⁺ Rt: 2.29 min |
| 42 2-{[(2-chloro-4- fluorophenyl)oxy]acetyl} hydrazinecarbothioamide | 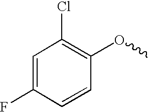 | 25 [(2-chloro-4- fluorophenyl) oxy]acetic acid | LC/MS: m/z 278 (M + H)⁺ Rt: 2.10 min |
| 43 2-{[(2-chloro-5- fluorophenyl)oxy]acetyl} hydrazinecarbothioamide | 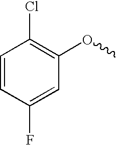 | 26 [(2-chloro-5- fluorophenyl) oxy]acetic acid | ¹H NMR (300 MHz, DMSO, ppm) δ: 8.77 (dd, 1 H, J = 1.52 Hz, 4.37 Hz), 8.54 (dd, 1 H, J = 1.35 Hz, 8.56 Hz), 8.00 (bs, 1 H), 7.67 (bs, 1 H), 7.50 (s, 1 H), 4.76 (s, 2 H). |
| 44 2-[(5,6,7,8-tetrahydro-1- naphthalenyloxy)acetyl] hydrazinecarbothioamide | 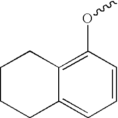 | 28 (5,6,7,8- tetrahydro-1- naphthalenyloxy) acetic acid | 1H NMR (300 MHz, DMSO, ppm) δ: 8.75 (dd, 1 H, J = 1.29 Hz, 4.58 Hz), 8.53 (dd, 1 H, J = 1.29 Hz, 8.56 Hz), 7.49 (s, 1 H), 4.65 (s, 2 H), 2.69 (m, 4 H), 1.70 (m, 4 H). |
| 45 2-[(1- naphthalenyloxy)acetyl] hydrazinecarbothioamide | 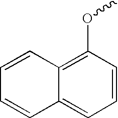 | 32 (1-naphthalenyloxy) acetic acid | LC/MS: m/z 276 (M + H)⁺ Rt: 2.36 min |
| 46 2-[3-(2- chlorophenyl)propanoyl] hydrazinecarbothioamide | 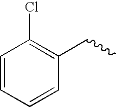 | Commercially available | LC/MS: m/z 258 (M + H)⁺ Rt: 2.10 min |
| 47 2-[3-(1- naphthalenyl)propanoyl] hydrazinecarbothioamide | 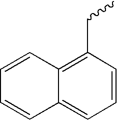 | Commercially available | LC/MS: m/z 274 (M + H)⁺ Rt: 2.52 min |
| 48 2-[(2-bromophenyl)acetyl] hydrazinecarbothioamide | 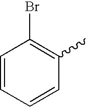 | Commercially available | LC/MS: m/z 290 (M + H)⁺ Rt: 1.73 min |
| 49 2-(1-benzothien-3-ylacetyl) hydrazinecarbothioamide | 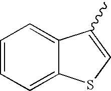 | Commercially available | Not isolated |
| 50 2-(3-thienylacetyl) hydrazinecarbothioamide | 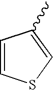 | Commercially available | Not isolated |
| 51 2-(5,6,7,8-tetrahydro-2- naphthalenylacetyl) hydrazinecarbothioamide | 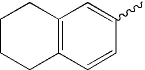 | Commercially available | LC/MS: m/z 264.14 (M + H)⁺ Rt: 2.39 min |
| 52 2-(3,4-dihydro-2H- chromen-6-ylacetyl) hydrazinecarbothioamide | 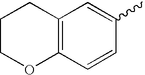 | Commercially available | LC/MS: m/z 266.2 (M + H)⁺ Rt: 1.87 min |
| 53 2-{[(2,4- dichlorophenyl)oxy]acetyl} hydrazinecarbothioamide | 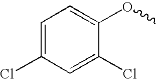 | 36 [(2,4- dichlorophenyl) oxy]acetic acid | LC/MS: m/z 294.1 (M + H)⁺ Rt: 2.36 min |

The following Intermediates were prepared using the generic reaction scheme (Scheme 11)

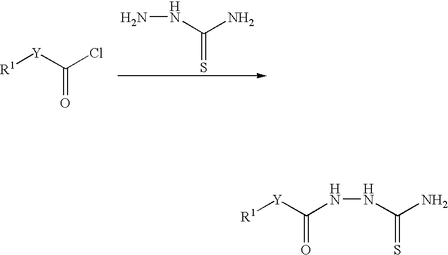

### Intermediate 54: 2-(Phenylacetyl)hydrazinecarbothioamide

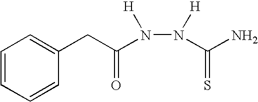

A solution of phenylacetyl chloride (5.27 mL, 0.04 mol.) in DMF (100 mL) was added at room temperature to a solution of thiosemicarbazide (3.64 g, 0.04 mol.) and pyridine (3.23 mL, 0.04 mol.) in DMF. After stirring for 8 hours, the mixture was poured into iced water and the pH was adjusted to 9 with ammonia. After extraction of the aqueous layer with ethyl acetate, the organic layer was washed with water, dried on Na₂SO₄ and after filtration was evaporated to dryness. Then the residue was recrystallized in ethyl acetate to give the title compound as a solid (1.878 g, 22%).

LC/MS: m/z 210 (M+H)⁺, Rt: 1.75 min.

The following Intermediates were similarly prepared by a method analogous to that described for Intermediate 54.

TABLE 4

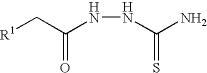

| Intermediate No. | R¹ | From: | Physical data |
|---|---|---|---|
| 55 2-(2-thienylacetyl) hydrazinecarbothioamide | 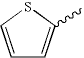 | Commercially available | LC/MS: m/z 214 (M − H)⁺ Rt: 2.74 min |
| 56 2-(2-naphthalenylacetyl) hydrazinecarbothioamide | 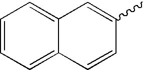 | Commercially available | ¹H NMR (300 MHz, DMSO, ppm) δ: 7.85 (m, 3 H), 7.78 (bs, 1 H), 7.48 (m, 3 H), 3.34 (s, 2 H). |

The following Intermediates were prepared using the generic reaction scheme (Scheme 11)

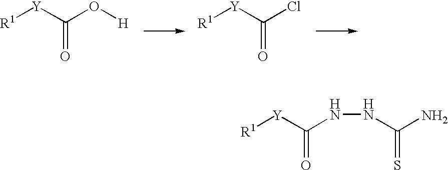

### Intermediate 57: (Method A) 2-{[(4-Chlorophenyl)oxy]acetyl}hydrazinecarbothioamide

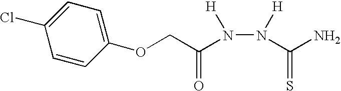

To a solution of [(4-chlorophenyl)oxy]acetic acid (Intermediate 20) (13.0 g, 0.07 mol.) in chloroform was slowly added thionyl chloride (7.5 mL, 0.1 mol.). The solution was refluxed for 4 hours. Then the solvent was evaporated under vacuum. The residue was dissolved in DMF then added at room temperature to a solution of thiosemicarbazide (7.28 g, 0.08 mol.) and pyridine (7.8 g, 0.1 mol.) in DMF. After stirring for 2 hours, the mixture was poured in to ice-water and the solid material was filtered and dried to give the title compound as a white solid (16 g, 88%).

LC/MS: m/z 260 (M+H)⁺, Rt: 2.69 min.

The following Intermediates were similarly prepared by a method analogous to that described for Intermediate 57.

TABLE 5

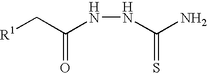

| | | From | |
|---|---|---|---|
| Intermediate No. | R¹ | Intermediate No. | Physical data |
| 58 2-{[(3,4-dichlorophenyl) oxy]acetyl} hydrazinecarbothioamide | 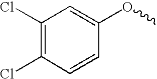 | 27 [(3,4-dichlorophenyl) oxy]acetic acid | LC/MS: m/z 294 (M − H)⁺ Rt: 2.72 min |

### Intermediate 59: (Method B) 2-{[(2-fluorophenyl)oxy]acetyl}hydrazinecarbothioamide

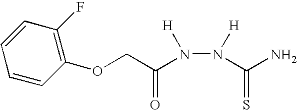

To a solution of [(2-fluorophenyl)oxy]acetic acid (Intermediate 21) (3 g, 0.018 mol.) in dichloromethane cooled at 0° C., was slowly added oxalyl chloride (3.07 mL, 0.035 mol.). After stirring for 2 hours at room temperature, the solvent was evaporated under vacuum. The residue was dissolved in DMF then added to a solution of thiosemicarbazide (1.63 g, 0.018 mol.) and pyridine (0.95 ml, 0.018 mol.) in DMF and cooled with an ice-water bath. After stirring for 4 hours, the mixture was poured into iced-water. The resulting solid material was filtered and dried to give the title compound (3 g, 67%).

LC/MS: m/z 244 (M+H)⁺, Rt: 2.13 min.

The following Intermediates were similarly prepared by a method analogous to that described for Intermediate 59.

TABLE 6

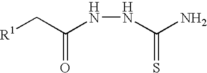

| | | From | |
|---|---|---|---|
| Intermediate No. | R¹ | Intermediate No. | Physical data |
| 60 2-{[(3-fluorophenyl) oxy]acetyl} hydrazinecarbothioamide | 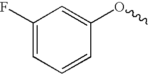 | 22 [(3-fluorophenyl) oxy]acetic acid | LC/MS: m/z 244 (M + H)⁺ Rt: 2.25 min |
| 61 2-{[(4-fluorophenyl) oxy]acetyl} hydrazinecarbothioamide | 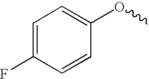 | 23 [(4-fluorophenyl) oxy]acetic acid | LC/MS: m/z 244 (M + H)⁺ Rt: 2.16 min |
| 62 2-(3-phenylpropanoyl) hydrazinecarbothioamide | 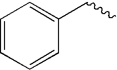 | Commercially available | ¹H NMR (300 MHz, DMSO, ppm) δ: 7.23 (m, 5 H), 2.83 (t, 2 H, J = 7.54 Hz), 2.42 (t, 2 H, J = 7.54 Hz. |
| 63 2-[(3,4- dichlorophenyl)acetyl] hydrazinecarbothioamide | 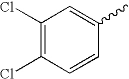 | Commercially available | LC/MS: m/z 278 (M + H)⁺ Rt: 2.53 min |
| 64 2-[(4-chlorophenyl)acetyl] hydrazinecarbothioamide | 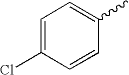 | Commercially available | LC/MS: m/z 244 (M + H)⁺ Rt: 2.23 min |
| 65 2-({[3- (trifluoromethyl)phenyl] oxy}acetyl) hydrazinecarbothioamide | 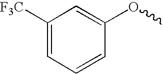 | 29 {[3-(trifluoromethyl) phenyl] oxy}acetic acid | LC/MS: m/z 294 (M + H)⁺ Rt: 2.76 min |
| 66 2-{[(3- chlorophenyl)oxy]acetyl} hydrazinecarbothioamide | 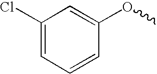 | 30 [(3-chlorophenyl) oxy]acetic acid | LC/MS: m/z 262 (M + H)⁺ Rt: 2.06 min |
| 67 2-({[2- (trifluoromethyl)phenyl] oxy}acetyl) hydrazinecarbothioamide | 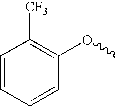 | 31 {[2-(trifluoromethyl) phenyl]oxy} acetic acid | LC/MS: m/z 294 (M + H)⁺ Rt: 2.72 min |
| 68 2-{[3- (trifluoromethyl)phenyl] acetyl} hydrazinecarbothioamide | 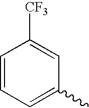 | 38 [3-(Trifluoromethyl) phenyl]acetic acid | ¹H NMR (300 MHz, DMSO, ppm) δ: 10.03 (bs, 1 H), 9.24 (bs, 1 H), 7.58 (m, 4 H), 3.6 (s, 2 H). |
| 69 2-[(2-chlorophenyl)acetyl] hydrazinecarbothioamide | 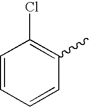 | Commercially available | ¹H NMR (300 MHz, DMSO, ppm) δ: 9.99 (bs, 1 H), 9.26 (bs, 1 H), 7.4 (m, 2 H), 7.26 (m, 2 H), 3.6 (s, 2 H). |
| 70 2-{[2- (trifluoromethyl)phenyl] acetyl} hydrazinecarbothioamide | 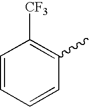 | Commercially available | ¹H NMR (300 MHz, DMSO, ppm) δ: 9.99 (s, 1 H), 9.26 (s, 1 H), 7.58-7.68 (m, 3 H), 7.42-7.46 (m, 3 H), 3.70 (s, 2 H). |
| 71 2-({[4- (methyloxy)phenyl]oxy} acetyl) hydrazinecarbothioamide | 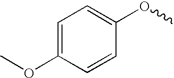 | 33 {[4-(methyloxy) phenyl] oxy}acetic acid | LC/MS: m/z 256 (M + H)⁺ Rt: 1.88 min |
| 72 2-[(2- biphenylyloxy)acetyl] hydrazinecarbothioamide | 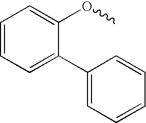 | 34 (2-biphenylyloxy) acetic acid | ¹H NMR (300 MHz, DMSO, ppm) δ: 7.6-6.9 (m, 9 H), 4.6 (s, 2 H). |
| 73 2-{[4- (trifluoromethyl)phenyl] acetyl} hydrazinecarbothioamide | 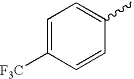 | Commercially available | ¹H NMR (300 MHz, DMSO, ppm) δ: 10.02 (s, 1 H), 9.24 (s, 1 H), 7.91 (m, 4 H), 7.51- 7.65 (m, 2 H), 3.59 (s, 2 H). |
| 74 2-[({5-chloro-2-[(2- methylpropyl)oxy]phenyl} oxy)acetyl] hydrazinecarbothioamide | 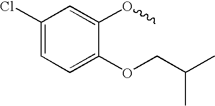 | 35 ({5-chloro-2-[(2- methylpropyl)oxy] phenyl}oxy)acetic acid | ¹H NMR (300 MHz, DMSO, ppm) δ: 7.75 (m, 3 H), 5.4 (s, 2 H), 4.5 (d, 2 H), 2.8 (m, 1 H), 1.80 (d, 6 H). |
| 75 2-[(4-fluorophenyl)acetyl] hydrazinecarbothioamide | 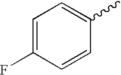 | Commercially available | LC/MS: m/z 228 (M + H)⁺ Rt: 3.19 min |
| 76 2-({[2-(methyloxy)phenyl] oxy}acetyl) hydrazinecarbothioamide | 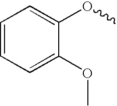 | 37 [(2,4- dichlorophenyl) oxy]acetic acid | ¹H NMR (300 MHz, DMSO, ppm) δ: 9.96 (s, 1 H), 9.26 (s, 1 H), 7.91 (s, 1 H), 7.52 (s, 1 H), 6.95 (m, 4 H), 4.52 (s, 2 H), 3.75 (s, 3 H). |

The following Intermediates were prepared using the generic reaction scheme (Scheme 8)

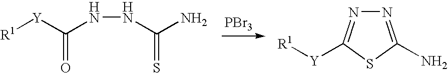

### Intermediate 77: 5-[(4-Chlorophenyl)methyl]-1,3,4-thiadiazol-2-amine

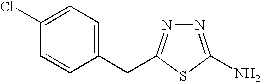

A mixture of the 2-[(4-chlorophenyl)acetyl]hydrazinecarbothioamide (Intermediate 64) (5 g, 0.020 mol.) and PBr₃ (30 mL, 0.146 mol.) was heated at 60° C. for 16 hours. Then the reaction was poured into ice-water and the pH was adjusted to 9 with ammonia. After filtration of the suspension, the solid material was dried to give the title compound as a solid (3.6 g, 79%).

LC/MS: m/z 226 (M+H)⁺, Rt: 3.55 min.

The following Intermediates were similarly prepared by a method analogous to that described for Intermediate 77.

TABLE 7

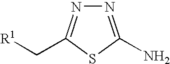

| | | From | |
|---|---|---|---|
| Intermediate No. | R¹ | Intermediate No. | Physical data |
| 78 5-(phenylmethyl)-1,3,4- thiadiazol-2-amine | 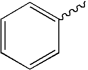 | 54 2-(phenylacetyl) hydrazine- carbothioamide | LC/MS: m/z 192 (M + H)⁺ Rt: 2.07 min |
| 79 5-[(3,4- dichlorophenyl)methyl]- 1,3,4-thiadiazol-2- amine | 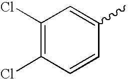 | 63 2-[(3,4- dichlorophenyl)acetyl] hydrazinecarbothioamide | LC/MS: m/z 260 (M + H)⁺ Rt: 3.89 min |
| 80 5-{[(4- chlorophenyl)oxy] methyl}-1,3,4- thiadiazol-2-amine | 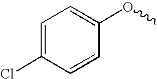 | 57 2-{[(4- chlorophenyl)oxy]acetyl} hydrazinecarbothioamide | LC/MS: m/z 242 (M + H)⁺ Rt: 3.79 min |
| 81 5-{[(2-fluorophenyl)oxy] methyl}-1,3,4- thiadiazol-2-amine | 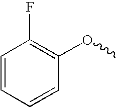 | 59 2-{[(2- fluorophenyl)oxy]acetyl} hydrazinecarbothioamide | LC/MS: m/z 226 (M + H)⁺ Rt: 3.39 min |
| 82 5-{[(3-fluorophenyl)oxy] methyl}-1,3,4- thiadiazol-2-amine | 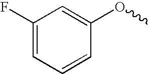 | 60 2-{[(3- fluorophenyl)oxy]acetyl} hydrazinecarbothioamide | LC/MS: m/z 226 (M + H)⁺ Rt: 3.47 min |
| 83 5-{[(4-fluorophenyl)oxy] methyl}-1,3,4- thiadiazol-2-amine | 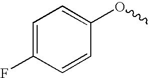 | 61 2-{[(4- fluorophenyl)oxy]acetyl} hydrazinecarbothioamide | LC/MS: m/z 226 (M + H)⁺ Rt: 3.42 min |
| 84 5-(2-thienylmethyl)- 1,3,4-thiadiazol-2- amine | 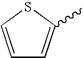 | 55 2-(2-thienylacetyl) hydrazinecarbothioamide | LC/MS: m/z 198 (M + H)⁺ Rt: 2.05 min |
| 85 5-{[(3,4- dichlorophenyl)oxy] methyl}-1,3,4- thiadiazol-2-amine | 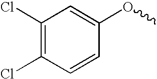 | 58 2-{[(3,4-dichlorophenyl) oxy]acetyl} hydrazinecarbothioamide | LC/MS: m/z 276 (M + H)⁺ Rt: 4.07 min |
| 86 5-{[(3- chlorophenyl)oxy[ methyl}-1,3,4- thiadiazol-2-amine | 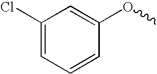 | 66 2-{[(3- chlorophenyl)oxy]acetyl} hydrazinecarbothioamide | LC/MS: m/z 242 (M + H)⁺ Rt: 3.70 min |
| 87 5-({[3- (trifluoromethyl)phenyl] oxy}methyl)-1,3,4- thiadiazol-2-amine | 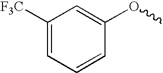 | 65 2-({[3-(trifluoromethyl) phenyl]oxy}acetyl) hydrazinecarbothioamide | LC/MS: m/z 276 (M + H)⁺ Rt: 3.98 min |
| 88 5-({[2- (trifluoromethyl)phenyl] oxy}methyl)-1,3,4- thiadiazol-2-amine | 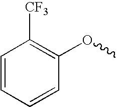 | 67 2-({[2-(trifluoromethyl) phenyl]oxy}acetyl) hydrazinecarbothioamide | LC/MS: m/z 276 (M + H)⁺ Rt: 3.88 min |
| 89 5-{[3- (trifluoromethyl)phenyl] methyl}-1,3,4- thiadiazol-2-amine | 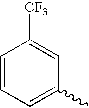 | 68 2-{[3-(trifluoromethyl) phenyl]acetyl} hydrazinecarbothioamide | ¹H NMR (300 MHz, DMSO, ppm) δ: 7.6 (m, 4H), 4.25 (s, 2H). |
| 90 5-[(2- chlorophenyl)methyl]- 1,3,4-thiadiazol-2- amine | 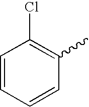 | 69 2-[(2-chlorophenyl)acetyl] hydrazinecarbothioamide | 1H NMR (300 MHz, DMSO, ppm) δ: 7.46-7.0 (m, 4H), 4.23 (s, 2H). |
| 91 5-{[2- (trifluoromethyl)phenyl] methyl}-1,3,4- thiadiazol-2-amine | 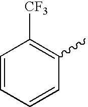 | 70 2-{[2-(trifluoromethyl) penyl]acetyl} hydrazinecarbothioamide | LC/MS: m/z 260 (M + H)⁺ Rt: 2.21 min |
| 92 5-[(2- biphenylyloxy)methyl]- 1,3,4-thiadiazol-2- amine | 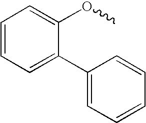 | 72 2-[(2- biphenylyloxy)acetyl] hydrazinecarbothioamide | ¹H NMR (300 MHz, DMSO, ppm) δ: 7.5- 7.0 (m, 11H), 5.25 (s, 2H). |
| 93 5-{[4- (trifluoromethyl)phenyl]methyl}-1,3,4- thiadiazol-2-amine | 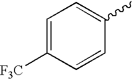 | 73 2-{[4-(trifluoromethyl) phenyl]acetyl} hydrazinecarbothioamide | ¹H NMR (300 MHz, DMSO, ppm) δ: 7.7 (d, 2H), 7.5 (d, 2H), 7.1 (m, 2H), 4.25 (s, 2H). |
| 94 5-[(4- fluorophenyl)methyl]- 1,3,4-thiadiazol-2- amine | 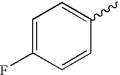 | 75 2-[(4-fluorophenyl)acetyl] hydrazinecarbothioamide | ¹H NMR (300 MHz, DMSO, ppm) δ: 7.2 (d, 2H), 7.1 (d, 2H), 7.00 (s, 2H), 4.1 (s, 2H). |

The following Intermediates were prepared using the generic reaction scheme (Scheme 8)

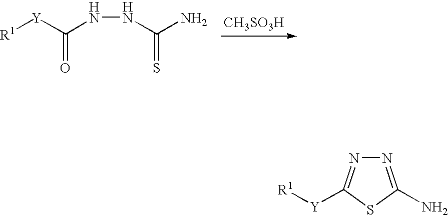

### Intermediate 95: 5-{[(2-Chlorophenyl)oxy]methyl}-1,3,4-thiadiazol-2-amine

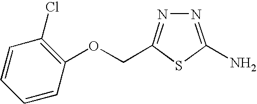

To a solution of 2-{[(2-Chlorophenyl)oxy]acetyl}hydrazinecarbothioamide (Intermediate 39) (36.6 g, 0.14 mol) in toluene (250 ml) was added drop by drop methane sulphonic acid (13.7 mL, 0.21 mol) and the reaction mixture was stirred at reflux for 2 hours. The solvent was evaporated. The residue was diluted with water and ammonia solution was added until pH=9. The formed precipitated was filtered and dried to give the title compound as a pale yellow solid (20.3 g, 60%).

LC/MS: m/z 242 (M+H)⁺, Rt: 2.57 min.

The following Intermediates were similarly prepared by a method analogous to that described for Intermediate 95.

TABLE 8

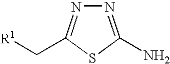

| | | From | |
|---|---|---|---|
| Intermediate No. | R¹ | Intermediate No. | Physical data |
| 96 5-(1H-indol-3- ylmethyl)-1,3,4- thiadiazol-2-amine | 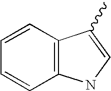 | 40 2-(1H-indol-3- ylacetyl)hydrazine- carbothioamide | LC/MS: m/z 231 (M + H)⁺, Rt: 2.07 min. |
| 97 5-(2- naphthalenylmethyl)- 1,3,4-thiadiazol-2- amine | 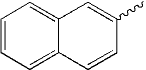 | 56 2-(2- naphthalenylacetyl) hydrazine- carbothioamide | ¹H NMR (300 MHz, DMSO, ppm) δ: 7.90 (m, 3H), 7.83 (bs, 1H), 7.52 (m, 2H), 7.44 (dd, 1H, J = 1.77 Hz, 8.32 Hz), 4.36 (s, 2H). |
| 98 5-(2-phenylethyl)- 1,3,4-thiadiazol-2- amine | 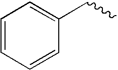 | 62 2-(3- phenylpropanoyl) hydrazine- carbothioamide | ¹H NMR (300 MHz, DMSO, ppm) δ: 7.19 (m, 5H), 3.04 (t, 2H, J = 7.27 Hz), 2.87 (t, 2H, J = 8.16 Hz). |
| 99 5-{[(2,5- dichlorophenyl)oxy] methyl}-1,3,4- thiadiazol-2-amine | 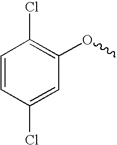 | 41 2-{[(2,5- dichlorophenyl)oxy] acetyl}hydrazine- carbothioamide | ¹H NMR (300 MHz, DMSO, ppm) δ: 7.49 (d, 1H, J = 8.56 Hz), 7.46 (d, 1H, J = 2.27 Hz), 7.09 (dd, 1H, J = 2.35 Hz, 8.47 Hz), 5.44 (s, 2H). |
| 100 5-{[(2-chloro-4- fluorophenyl)oxy] methyl}-1,3,4- thiadiazol-2-amine | 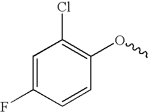 | 42 2-{[(2-chloro-4- fluorophenyl)oxy] acetyl}hydrazine- carbothioamide | ¹H NMR (300 MHz, DMSO, ppm) δ: 7.47 (dd, 1H, J = 3.15 Hz, 8.41 Hz), 7.33 (m, 1H), 7.21 (td, 1H, J = 3.15 Hz, 8.10 Hz), 5.37 (s, 2H). |
| 101 5-{[(2-chloro-5- fluorophenyl)oxy]meth- yl}-1,3,4-thiadiazol-2- amine | 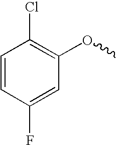 | 43 2-{[(2-chloro-5- fluorophenyl)oxyl]acet- yl}hydrazinecarbothio- amide | ¹H NMR (300 MHz, DMSO, ppm) δ: 7.50 (dd, 1H, 8.93 Hz, 6.09 Hz), 7.30 (dd, 1H, J = 2.81 Hz, 10.48 Hz), 6.88 (dd, 1H, J = 2.94 Hz, 8.31 Hz), 5.42 (s, 2H). |
| 102 5-[(5,6,7,8-tetrahydro- 1-naphthalenyloxy) methyl]-1,3,4- thiadiazol-2-amine | 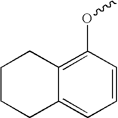 | 44 2-[(5,6,7,8- tetrahydro-1- naphthalenyloxy) acetyl]hydrazine- carbothioamide | ¹H NMR (300 MHz, DMSO, ppm) δ: 7.04 (t, 1H, J = 7.96 Hz), 6.86 (d, 1H, J = 7.96 Hz), 6.70 (d, 1H, J = 7.62 Hz), 5.24 (s, 2H), 2.69 (m, 2H), 2.56 (m, 2H), 1.70 (m, 4H). |
| 103 5-[(1-naphthalenyloxy) methyl]-1,3,4- thiadiazol-2-amine | 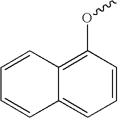 | 45 2-[(1- naphthalenyloxy) acetyl]hydrazine- carbothioamide | LC/MS: m/z 258 (M + H)⁺ Rt: 2.69 min |
| 104 5-[(2- chlorophenyl)ethyl]- 1,3,4-thiadiazol-2- amine | 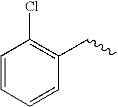 | 46 2-[3-(2-chlorophenyl) propanoyl]hydrazine- carbothioamide | LC/MS: m/z 240 (M + H)⁺ Rt: 2.49 min |
| 105 5-[2-(1- naphthalenyl)ethyl]- 1,3,4-thiadiazol-2- amine | 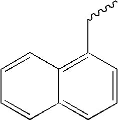 | 47 2-[3-(1-naphthalenyl) propanoyl]hydrazine- carbothioamide | LC/MS: m/z 256 (M + H)⁺ Rt: 2.88 min |
| 106 5-[(2- bromophenyl)methyl]- 1,3,4-thiadiazol-2- amine | 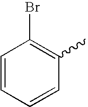 | 48 2-[(2- bromophenyl)acetyl] hydrazinecarbothio- amide | LC/MS: m/z 272 (M + H)⁺ Rt: 2.36 min |
| 107 5-(1-benzothien-3- ylmethyl)-1,3,4- thiadiazol-2-amine | 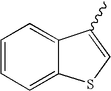 | 49 2-(1-benzothien-3- ylacetyl) hydrazine- carbothioamide | ¹H NMR (300 MHz, DMSO, ppm) δ: 8.00 (m, 1H), 7.84 (m, 1H), 7.64 (s, 1H), 7.40 (m, 2H), 4.43 (s, 2H). |
| 108 5-(3-thienylmethyl)- 1,3,4-thiadiazol-2- amine | 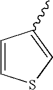 | 50 2-(3-thienylacetyl) hydrazine- carbothioamide | LC/MS: m/z 198 (M + H)⁺ Rt: 1.93 min |
| 109 5-({[4- (methyloxy)phenyl] oxy}methyl)-1,3,4- thiadiazol-2-amine | 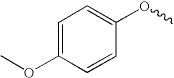 | 71 2-({[4-(methyloxy) phenyl]oxy}acetyl) hydrazine- carbothioamide | ¹H NMR (300 MHz, DMSO, ppm) δ: 6.95 (d, 2H), 6.85 (d, 2H), 5.2 (s, 2H), 3.7 (s, 3H). |
| 110 5-[({5-chloro-2-[(2- methylpropyl)oxy]phen- yl}oxy)methyl]-1,3,4- thiadiazol-2-amine | 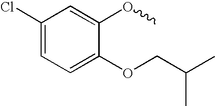 | 74 2-[({5-chloro-2-[(2- methylpropyl)oxy] phenyl}oxy)acetyl] hydrazine- carbothioamide | ¹H NMR (300 MHz, DMSO, ppm) δ: 7.3 (m, 2H), 7.2 (s, 1H), 7.00 (s, 2H), 5.3 (s, 2H), 3.75 (d, 2H), 2.00 (m, 1H), 1.00 (d, 6H). |
| 111 5-({[2- (methyloxy)phenyl] oxy}methyl)-1,3,4- thiadiazol-2-amine | 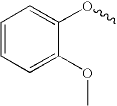 | 76 2-({[2- (methyloxy)phenyl] oxy}acetyl)hydrazine- carbothioamide | ¹H NMR (300 MHz, DMSO, ppm) δ: 7.25 (s, 2H), 7.00 (m, 4H), 5.25 (s, 2H), 3.75 (s, 3H). |
| 112 5-(5,6,7,8-tetrahydro- 2-naphthalenylmethyl)- 1,3,4-thiadiazol-2- amine | 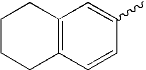 | 51 2-(5,6,7,8-tetrahydro- 2-naphthalenylacetyl) hydrazine- carbothioamide | LC/MS: m/z 246 (M + H)⁺ Rt: 2.53 min |
| 113 5-(3,4-dihydro-2H- chromen-6-ylmethyl)- 1,3,4-thiadiazol-2- amine | 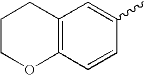 | 52 2-(3,4-dihydro-2H- chromen-6- ylacetyl)hydrazine- carbothioamide | LC/MS: m/z 248 (M + H)⁺ Rt: 2.12 min |
| 114 5-{[(2,4- dichlorophenyl)oxy] methyl}-1,3,4- thiadiazol-2-amine | 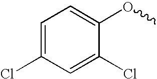 | 53 2-{[(2,4- dichlorophenyl)oxy] acetyl}hydrazine- carbothioamide | LC/MS: m/z 276 (M + H)⁺ Rt: 2.51 min |

The following Intermediates were prepared using the generic reaction scheme (Scheme 9)

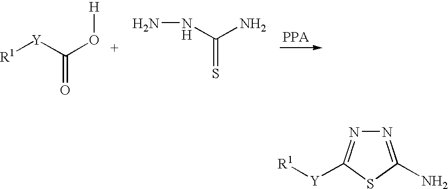

### Intermediate 115: 5-(Cyclohexylmethyl)-1,3,4-thiadiazol-2-amine

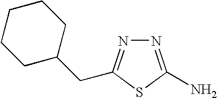

Hydrazinecarbothioamide (0.32 g, 3.5 mmol) in PPA (50 g) was heated at 110° C. until dissolution. Cyclohexylacetic acid (0.5 g, 3.5 mmol.) was added and the reaction mixture was heated at 110° C. for 2 hours. After cooling, the reaction was poured in ice and aqueous ammonia was added until pH=9. The formed precipitated was filtered and dried to give the title compound as a white solid (0.4 g, 69.4%).

LC/MS: m/z 198 (M+H)⁺, Rt: 2.49 min

### Intermediate 116: 1,1,1-Dimethylethyl[5-(1H-indol-3-ylmethyl)-1,3,4-thiadiazol-2-yl]carbamate

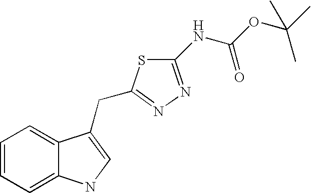

A solution of 5-(1H-indol-3-ylmethyl)-1,3,4-thiadiazol-2-amine, 5-(1H-indol-3-ylmethyl)-1,3,4-thiadiazol-2-amine (Intermediate 96) (500 mg, 2.2 mmol), anhydride boc (521 mg, 2.4 mmol) and triethylamine (300 μl, 2.2 mmol) in THF was stirred at 50° C. overnight. The mixture was evaporated. The residue was diluted with dichloromethane, washed with water and the organic phase was dried over Na₂SO₄ to give after evaporation the title compound as brown crystals (550 mg, 76%).

LC/MS: m/z 331.2 (M+H)⁺, Rt: 2.92 min

### Intermediate 117: 1,1-Dimethylethyl {5-[(1-methyl-1H-indol-3-yl)methyl]-1,3,4-thiadiazol-2-yl}carbamate

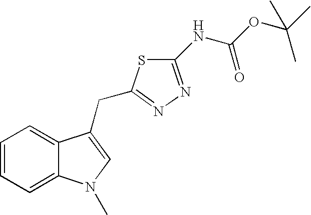

To a solution of 1,1-dimethylethyl [5-(1H-indol-3-ylmethyl)-1,3,4-thiadiazol-2-yl]carbamate, (Intermediate 116) (550 mg, 1.66 mmol) and NaH 60% (133 mg, 3.33 mmol) in THF stirred for 1 hour at room temperature, was added methyl iodide (125 μl, 2 mmol). The reaction mixture was stirred at room temperature for 4 hours. The mixture was then heated at 50° C. overnight and NaH 60% (133 mg, 3.33 mmol) and methyl iodide (52 μl, 0.83 mmol) were added and the mixture was stirred at 50° C. for a further night. The mixture was hydrolysed and evaporated under reduced pressure. The residue was purified by flash column chromatography eluting with a gradient DCM 100% to DCM/MeOH:60/40 to give the title compound as yellow crystals (100 mg, 17%).

LC/MS: m/z 345.2 (M+H)⁺, Rt: 3.18 min

### Intermediate 118: 5-[(1-Methyl-1H-indol-3-yl)methyl]-1,3,4-thiadiazol-2-amine

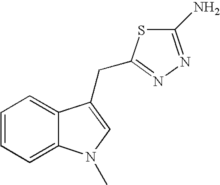

HCl(g) was bubbled at 0° C. in EtOAc and 1,1-dimethylethyl {5-[(1-methyl-1H-indol-3-yl)methyl]-1,3,4-thiadiazol-2-yl}carbamate (Intermediate 117) (100 mg, 0.3 mmol) was added. The reaction mixture was stirred at room temperature overnight. The mixture was evaporated and the residue was recrystallised with acetonitrile to give the title compound as a solid (40 mg, 55%).

LC/MS: m/z 245.08 (M+H)⁺, Rt: 2.37 min

### Intermediate 119: 5-[(2′-Chloro-2-biphenylyl)methyl]-1,3,4-thiadiazol-2-amine

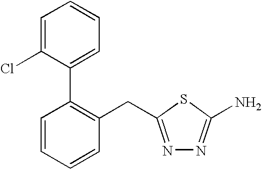

A solution of 5-[(2-bromophenyl)methyl]-1,3,4-thiadiazol-2-amine (Intermediate 106) (500 mg, 1.75 mmol), Pd(PPh₃)₄ (50 mg), Na₂CO₃ 2M (3.5 ml, 7 mmol) and 2-chlorophenylboronic acid (354 mg, 2.3 mmol) in DME was stirred at reflux for 48 hours. The mixture was evaporated. The residue was diluted with dichloromethane, washed with water. The organic phase was dried over Na₂SO₄, filtered and evaporated. The obtained solid was recrystallised with acetonitrile to give the title compound as a solid (125 mg, 24%).

LC/MS: m/z 302.02 (M+H)⁺, Rt: 2.98 min.

The following Intermediates were prepared using the generic reaction scheme (Scheme 1):

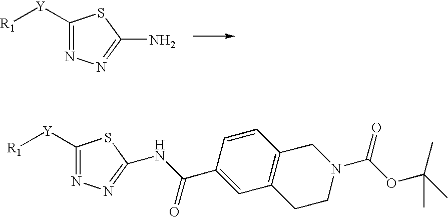

### Intermediate 120: 1,1-Dimethylethyl 6-{[(5-{[(2-chlorophenyl)oxy]methyl}-1,3,4-thiadiazol-2-yl)amino]carbonyl}-3,4-dihydro-2(1H)-isoquinolinecarboxylate

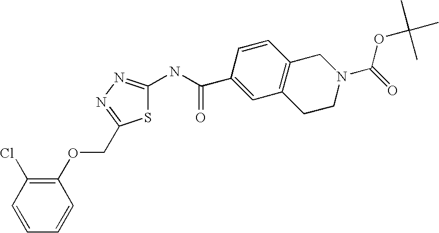

A solution of 2-{[(1,1-dimethylethyl)oxy]carbonyl}-1,2,3,4-tetrahydro-6-isoquinolinecarboxylic acid (14.3 g, 52 mmol), HATU (29.5 g, 77.6 mmol), DIPEA (14.6 mL, 62 mmol) in DMF was stirred at room temperature for 1 hour. 5-{[(2-chlorophenyl)oxy]methyl}-1,3,4-thiadiazol-2-amine, (Intermediate 95) (15 g, 62 mmol) was added and the mixture was stirred at room temperature overnight. The DMF was evaporated under reduced pressure and the residue was dissolved in EtOAc. The organic phase was then washed with water and filtered to eliminate an insoluble. The aqueous phase was re-extracted with EtOAc, and the organic phase was dried over sodium sulphate, filtered and evaporated under reduced pressure. The residue was then diluted with DCM and the insoluble was filtered. All the organic phases were combined, dried over sodium sulphate, filtered and evaporated under reduced pressure to give the title compound (14 g, 62%).

¹H NMR (300 MHz, DMSO, ppm) δ: 7.96 (s, 1H), 7.93 (d, 1H), 7.48 (d, 1H), 7.36 (m, 3H), 7.04 (m, 1H), 5.64 (s, 2H), 4.59 (s, 2H), 3.60 (t, 2H), 2.86 (t, 2H), 1.44 (s, 9H).

The following Intermediates were similarly prepared by a method analogous to that described for Intermediate 120.

TABLE 9

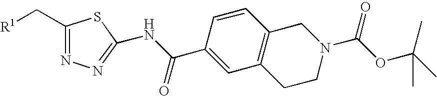

| | | From Intermediate | |
|---|---|---|---|
| Intermediate No. | R¹ | No. | Physical data |
| 121 1,1-dimethylethyl 6-({[5-(1- naphthalenylmethyl)-1,3,4- thiadiazol-2-yl]amino}carbonyl)- 3,4-dihydro-2(1H)- isoquinolinecarboxylate | 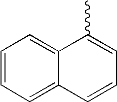 | Commercially available | LC/MS: m/z 501 (M + H)⁺ Rt: 3.69 min. |
| 122 1,1-dimethylethyl 6-({[5-(2- thienylmethyl)-1,3,4-thiadiazol- 2-yl]amino}carbonyl)-3,4- dihydro-2(1H)- isoquinolinecarboxylate | 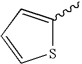 | 84 5-(2- thienylmethyl)- 1,3,4-thiadiazol-2- amine | LC/MS: m/z 457 (M + H)⁺ Rt: 3.14 min |
| 123 1,1-dimethylethyl 6-({[5-(2- naphthalenylmethyl)-1,3,4- thiadiazol-2-yl]amino}carbonyl)- 3,4-dihydro-2(1H)- isoquinolinecarboxylate | 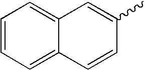 | 97 5-(2- naphthalenylmeth- yl)-1,3,4-thiadiazol- 2-amine | LC/MS: m/z 501 (M + H)⁺ Rt: 3.89 min |
| 124 1,1-dimethylethyl 6-({[5- (cyclohexylmethyl)-1,3,4- thiadiazol-2-yl]amino}carbonyl)- 3,4-dihydro-2(1H)- isoquinolinecarboxylate | 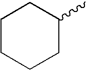 | 115 5- (Cyclohexylmethyl)- 1,3,4-thiadiazol-2- amine | ¹H NMR (300 MHz, DMSO, ppm) δ: 7.8 (m, 2H), 7.3 (d, 1H), 7.0 (s, 1H), 4.6 (brs, 2H), 3.6 (t, 2H), 2.85 (m, 4H), 1.55-1.8 (m, 11H), 1.45 (s, 3H). |
| 125 1,1-dimethylethyl 6-({[5-(2- phenylethyl)-1,3,4-thiadiazol-2- yl]amino}carbonyl)-3,4-dihydro- 2(1H)-isoquinolinecarboxylate | 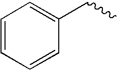 | 98 5-(2-phenylethyl)- 1,3,4-thiadiazol-2- amine | LC/MS: m/z 465 (M + H)⁺ Rt: 3.73 min |
| 126 1,1-dimethylethyl 6-({[5-(1H- indol-3-ylmethyl)-1,3,4- thiadiazol-2-yl]amino}carbonyl)- 3,4-dihydro-2(1H)- isoquinolinecarboxylate | 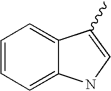 | 96 5-(1H-indol-3- ylmethyl)-1,3,4- thiadiazol-2-amine | LC/MS: m/z 278 (M + H)⁺ Rt: 3.30 min |
| 127 1,1-dimethylethyl 6-{[(5-{[(2,5- dichlorophenyl)oxyl]methyl}- 1,3,4-thiadiazol-2- yl)amino]carbonyl}-3,4-dihydro- 2(1H)-isoquinolinecarboxylate | 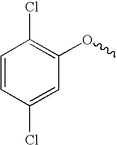 | 99 5-{[(2,5- dichlorophenyl)oxy] methyl}-1,3,4- thiadiazol-2-amine | LC/MS: m/z 535 (M + H)⁺ Rt: 4.02 min |
| 128 1,1-dimethylethyl 6-[({5-[(2- bromophenyl)methyl]-1,3,4- thiadiazol-2-yl}amino)carbonyl]- 3,4-dihydro-2(1H)- isoquinolinecarboxylate | 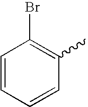 | 106 5-[(2- bromophenyl)meth- yl]-1,3,4-thiadiazol- 2-amine | LC/MS: m/z 530 (M + H)⁺ Rt: 3.83 min |
| 129 1,1-dimethylethyl 6-({[5-({[2- (trifluoromethyl)phenyl]oxy}meth- yl)-1,3,4-thiadiazol-2- yl]amino}carbonyl)-3,4-dihydro- 2(1H)-isoquinolinecarboxylate | 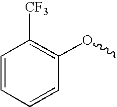 | 88 5-({[2- (trifluoromethyl) phenyl]oxy}methyl)- 1,3,4-thiadiazol-2- amine | LC/MS: m/z 535 (M + H)⁺ Rt: 3.70 min |
| 130 1,1-dimethylethyl 6-{[(5-{[(2- fluorophenyl)oxy]methyl}-1,3,4- thiadiazol-2-yl)amino]carbonyl}- 3,4-dihydro-2(1H)- isoquinolinecarboxylate | 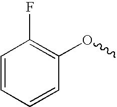 | 81 -{[(2- fluorophenyl)oxy] methyl}-1,3,4- thiadiazol-2-amine | LC/MS: m/z 485.2 (M + H)⁺ Rt: 3.71 min |
| 131 1,1-dimethylethyl 6-{[(5-{[(3- fluorophenyl)oxy]methyl}-1,3,4- thiadiazol-2-yl)amino]carbonyl}- 3,4-dihydro-2(1H)- isoquinolinecarboxylate | 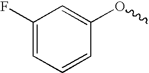 | 82 5-{[(3- fluorophenyl)oxy] methyl}-1,3,4- thiadiazol-2-amine | LC/MS: m/z 485.3 (M + H)⁺ Rt: 3.74 min |
| 132 1,1-dimethylethyl 6-{[(5-{[(4- fluorophenyl)oxy]methyl}-1,3,4- thiadiazol-2-yl)amino]carbonyl}- 3,4-dihydro-2(1H)- isoquinolinecarboxylate | 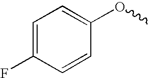 | 83 5-{[(4- fluorophenyl)oxy] methyl}-1,3,4- thiadiazol-2-amine | LC/MS: m/z 485 (M + H)⁺ Rt: 3.49 min |
| 133 1,1-dimethylethyl 6-({[5- (phenylmethyl)-1,3,4-thiadiazol- 2-yl]amino}carbonyl)-3,4- dihydro-2(1H)- isoquinolinecarboxylate | 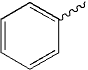 | 78 5-(phenylmethyl)- 1,3,4-thiadiazol-2- amine | LC/MS: m/z 451 (M + H)⁺ Rt: 3.47 min |
| 134 1,1-dimethylethyl 6-[({5-[(4- chlorophenyl)methyl]-1,3,4- thiadiazol-2-yl}amino)carbonyl]- 3,4-dihydro-2(1H)- isoquinolinecarboxylate | 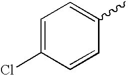 | 77 5-[(4- chlorophenyl)meth- yl]-1,3,4-thiadiazol- 2-amine | LC/MS: m/z 485 (M + H)⁺ Rt: 3.65 min |
| 135 1,1-dimethylethyl 6-[({5-[(3,4- dichlorophenyl)methyl]-1,3,4- thiadiazol-2-yl}amino)carbonyl]- 3,4-dihydro-2(1H)- isoquinolinecarboxylate | 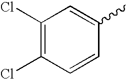 | 79 5-[(3,4- dichlorophenyl) methyl]-1,3,4- thiadiazol-2-amine | LC/MS: m/z 519 (M + H)⁺ Rt: 3.81 min. |
| 136 1,1-dimethylethyl 6-{[(5-{[(3- chlorophenyl)oxy]methyl}-1,3,4- thiadiazol-2-yl)amino]carbonyl}- 3,4-dihydro-2(1H)- isoquinolinecarboxylate | 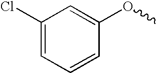 | 86 5-{[(3- chlorophenyl)oxy] methyl}-1,3,4- thiadiazol-2-amine | LC/MS: m/z 501 (M + H)⁺ Rt: 3.95 min |
| 137 1,1-dimethylethyl 6-{[(5-{[(3,4- dichlorophenyl)oxy]methyl}- 1,3,4-thiadiazol-2- yl)amino]carbonyl}-3,4-dihydro- 2(1H)-isoquinolinecarboxylate | 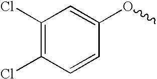 | 85 5-{[(3,4- dichlorophenyl)oxy] methyl}-1,3,4- thiadiazol-2-amine | LC/MS: m/z 535 (M + H)⁺ Rt: 3.89 min |
| 138 1,1-dimethylethyl 6-({[5-({[3- (trifluoromethyl)phenyl]oxy}meth- yl)-1,3,4-thiadiazol-2- yl]amino}carbonyl)-3,4-dihydro- 2(1H)-isoquinolinecarboxylate | 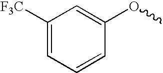 | 87 5-({[3- (trifluoromethyl) phenyl]oxy}methyl)- 1,3,4-thiadiazol-2- amine | LC/MS: m/z 535 (M + H)⁺ Rt: 3.97 min |
| 139 1,1-dimethylethyl 6-{[(5-{[(4- chlorophenyl)oxy]methyl}-1,3,4- thiadiazol-2-yl)amino]carbonyl}- 3,4-dihydro-2(1H)- isoquinolinecarboxylate | 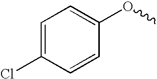 | 80 5-{[(4- chlorophenyl)oxy] methyl}-1,3,4- thiadiazol-2-amine | LC/MS: m/z 501 (M + H)⁺ Rt: 3.73 min. |
| 140 1,1-dimethylethyl 6-{[(5-{[(2- chloro-4- fluorophenyl)oxy]methyl}-1,3,4- thiadiazol-2-yl)amino]carbonyl}- 3,4-dihydro-2(1H)- isoquinolinecarboxylate | 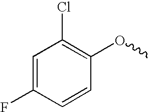 | 100 5-{[(2-chloro-4- fluorophenyl)oxy] methyl}-1,3,4- thiadiazol-2-amine | LC/MS: m/z 519 (M + H)⁺ Rt: 3.89 min. |
| 141 1,1-dimethylethyl 6-{[(5-{[(2- chloro-5- fluorophenyl)oxy]methyl}-1,3,4- thiadiazol-2-yl)amino]carbonyl}- 3,4-dihydro-2(1H)- isoquinolinecarboxylate | 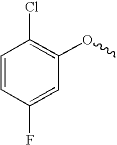 | 101 5-{[(2-chloro-5- fluorophenyl)oxy] methyl}-1,3,4- thiadiazol-2-amine | LC/MS: m/z 519 (M + H)⁺ Rt: 3.90 min. |
| 142 1,1-dimethylethyl 6-({[5-(1- benzothien-3-ylmethyl)-1,3,4- thiadiazol-2-yl]amino}carbonyl)- 3,4-dihydro-2(1H)- isoquinolinecarboxylate | 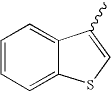 | 107 5-(1-benzothien-3- ylmethyl)-1,3,4- thiadiazol-2-amine | LC/MS: m/z 507 (M + H)⁺ Rt: 3.88 min. |
| 143 1,1-dimethylethyl 6-({[5-(3- thienylmethyl)-1,3,4-thiadiazol- 2-yl]amino}carbonyl)-3,4- dihydro-2(1H)- isoquinolinecarboxylate | 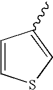 | 108 5-(3- thienylmethyl)- 1,3,4-thiadiazol-2- amine | LC/MS: m/z 457 (M + H)⁺ Rt: 3.61 min. |
| 144 1,1-dimethylethyl 6-[({5-[2-(1- naphthalenyl)ethyl]-1,3,4- thiadiazol-2-yl}amino)carbonyl]- 3,4-dihydro-2(1H)- isoquinolinecarboxylate | 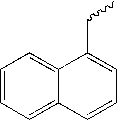 | 105 5-[2-(1- naphthalenyl)ethyl]- 1,3,4-thiadiazol-2- amine | LC/MS: m/z 515 (M + H)⁺ Rt: 4.00 min. |
| 145 1,1-dimethylethyl 6-[({5-[2-(2- chlorophenyl)ethyl]-1,3,4- thiadiazol-2-yl}amino)carbonyl]- 3,4-dihydro-2(1H)- isoquinolinecarboxylate | 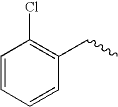 | 104 5-[2-(2- chlorophenyl)ethyl]- 1,3,4-thiadiazol-2- amine | LC/MS: m/z 500 (M + H)⁺ Rt: 3.91 min. |
| 146 1,1-dimethylethyl 6-[({5- [(5,6,7,8-tetrahydro-1- naphthalenyloxy)methyl]-1,3,4- thiadiazol-2-yl}amino)carbonyl]- 3,4-dihydro-2(1H)- isoquinolinecarboxylate | 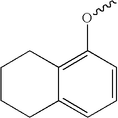 | 102 5-[(5,6,7,8- tetrahydro-1- naphthalenyloxy) methyl]-1,3,4- thiadiazol-2-amine | LC/MS: m/z 521 (M + H)⁺ Rt: 4.01 min. |
| 147 1,1-dimethylethyl 6-[({5-[(1- naphthalenyloxy)methyl]-1,3,4- thiadiazol-2-yl}amino)carbonyl]- 3,4-dihydro-2(1H)- isoquinolinecarboxylate | 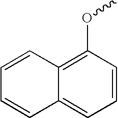 | 103 5-[(1- naphthalenyloxy) methyl]-1,3,4- thiadiazol-2-amine | LC/MS: m/z 517 (M + H)⁺ Rt: 3.94 min. |
| 148 1,1-dimethylethyl 6-{[(5-{[3- (trifluoromethyl)phenyl]methyl}- 1,3,4-thiadiazol-2- yl)amino]carbonyl}-3,4-dihydro- 2(1H)-isoquinolinecarboxylate | 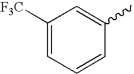 | 89 5-{[3- (trifluoromethyl) phenyl]methyl}-1,3,4- thiadiazol-2-amine | LC/MS: m/z 519.2 (M + H)⁺ Rt: 3.67 min. |
| 149 1,1-dimethylethyl 6-[({5-[(2- chlorophenyl)methyl]-1,3,4- thiadiazol-2-yl}amino)carbonyl]- 3,4-dihydro-2(1H)- isoquinolinecarboxylate | 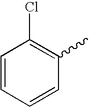 | 90 5-[(2- chlorophenyl)meth- yl]-1,3,4-thiadiazol- 2-amine | LC/MS: m/z 485.2 (M + H)⁺ Rt: 3.61 min. |
| 150 1,1-dimethylethyl 6-{[(5-{[2- (trifluoromethyl)phenyl]methyl}- 1,3,4-thiadiazol-2- yl)amino]carbonyl}-3,4-dihydro- 2(1H)-isoquinolinecarboxylate | 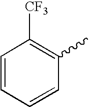 | 91 5-{[2- (trifluoromethyl) phenyl]methyl}-1,3,4- thiadiazol-2-amine | LC/MS: m/z 519.1 (M + H)⁺ Rt: 3.65 min. |
| 151 1,1-dimethylethyl 6-({[5-({[4- (methyloxy)phenyl]oxy}methyl)- 1,3,4-thiadiazol-2- yl]amino}carbonyl)-3,4-dihydro- 2(1H)-isoquinolinecarboxylate | 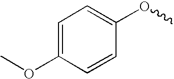 | 109 5-({[4- (methyloxy)phenyl] oxy}methyl)-1,3,4- thiadiazol-2-amine | LC/MS: m/z 497.15 (M + H)⁺ Rt: 3.58 min. |
| 152 1,1-dimethylethyl 6-[({5-[(2- biphenylyloxy)methyl]-1,3,4- thiadiazol-2-yl}amino)carbonyl]- 3,4-dihydro-2(1H)- isoquinolinecarboxylate | 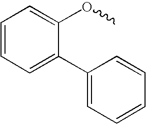 | 92 5-[(2- biphenylyloxy)meth- yl]-1,3,4-thiadiazol- 2-amine | LC/MS: m/z 543.2 (M + H)⁺ Rt: 3.83 min. |
| 153 1,1-dimethylethyl 6-{[(5-{[4- (trifluoromethyl)phenyl]methyl}- 1,3,4-thiadiazol-2- yl)amino]carbonyl}-3,4-dihydro- 2(1H)-isoquinolinecarboxylate | 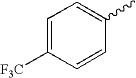 | 93 5-{[4- (trifluoromethyl) phenyl]methyl}-1,3,4- thiadiazol-2-amine | LC/MS: m/z 519.2 (M + H)⁺ Rt: 3.68 min. |
| 154 1,1-dimethylethyl 6-[({5-[({5- chloro-2-[(2- methylpropyl)oxy]phenyl}oxy) methyl]-1,3,4-thiadiazol-2- yl}amino)carbonyl]-3,4-dihydro- 2(1H)-isoquinolinecarboxylate | 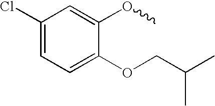 | 110 5- [({5-chloro-2-[(2- methylpropyl)oxy] phenyl}oxy)methyl]- 1,3,4-thiadiazol-2- amine | LC/MS: m/z 573.2 (M + H)⁺ Rt: 4.06 min. |
| 155 1,1-dimethylethyl 6-[({5-[(4- fluorophenyl)methyl]-1,3,4- thiadiazol-2-yl}amino)carbonyl]- 3,4-dihydro-2(1H)- isoquinolinecarboxylate | 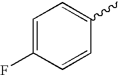 | 94 5-[(4- fluorophenyl)meth- yl]-1,3,4-thiadiazol- 2-amine | LC/MS: m/z 469.16 (M + H)⁺ Rt: 3.49 min. |
| 156 1,1-dimethylethyl 6-({[5-({[2- (methyloxy)phenyl]oxy}methyl)- 1,3,4-thiadiazol-2- yl]amino}carbonyl)-3,4-dihydro- 2(1H)-isoquinolinecarboxylate | 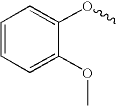 | 111 5-({[2- (methyloxy)phenyl] oxy}methyl)-1,3,4- thiadiazol-2-amine | LC/MS: m/z 497.1 (M + H)⁺ Rt: 3.40 min. |
| 157 1,1-dimethylethyl 6-[({5-[(1- methyl-1H-indol-3-yl)methyl]- 1,3,4-thiadiazol-2- yl}amino)carbonyl]-3,4-dihydro- 2(1H)-isoquinolinecarboxylate | 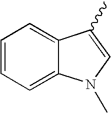 | 118 5-[(1-methyl-1H- indol-3-yl)methyl]- 1,3,4-thiadiazol-2- amine | LC/MS: m/z 504.14 (M + H)⁺ Rt: 3.60 min. |
| 158 1,1-dimethylethyl 6-({[5-(3- pyridinylmethyl)-1,3,4-thiadiazol- 2-yl]amino}carbonyl)-3,4- dihydro-2(1H)- isoquinolinecarboxylate | 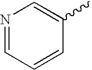 | Commercially available | LC/MS: m/z 452.09 (M + H) Rt: 2.96 min. |
| 159 1,1-dimethylethyl 6-({[5-(5,6,7,8- tetrahydro-2- naphthalenylmethyl)-1,3,4- thiadiazol-2-yl]amino}carbonyl)- 3,4-dihydro-2(1H)- isoquinolinecarboxylate | 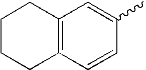 | 112 5-(5,6,7,8- tetrahydro-2- naphthalenylmethyl)- 1,3,4-thiadiazol- 2-amine | LC/MS: m/z 505.13 (M + H)⁺ Rt: 4.01 min. |
| 160 1,1-dimethylethyl 6-({[5-(3,4- dihydro-2H-chromen-6- ylmethyl)-1,3,4-thiadiazol-2- yl]amino}carbonyl)-3,4-dihydro- 2(1H)-isoquinolinecarboxylate | 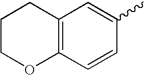 | 113 5-(3,4-dihydro-2H- chromen-6- ylmethyl)-1,3,4- thiadiazol-2-amine | LC/MS: m/z 507.1 (M + H)⁺ Rt: 3.52 min. |
| 161 1,1-dimethylethyl 6-{[(5-{2-[(2- chlorophenyl)oxy]ethyl}-1,3,4- thiadiazol-2-yl)amino]carbonyl}- 3,4-dihydro-2(1H)- isoquinolinecarboxylate | 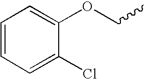 | Commercially available | LC/MS: m/z 515.15 (M + H)⁺ Rt: 3.67 min. |
| 162 1,1-dimethylethyl 6-{[(5-{[(2,4- dichlorophenyl)oxy]methyl}- 1,3,4-thiadiazol-2- yl)amino]carbonyl}-3,4-dihydro- 2(1H)-isoquinolinecarboxylate | 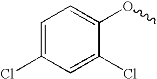 | 114 5-{[(2,4- dichlorophenyl)oxy] methyl}-1,3,4- thiadiazol-2-amine | LC/MS: m/z 535.05 (M + H)⁺ Rt: 3.85 min. |
| 163 1,1-dimethylethyl 6-[({5-[(2′- chloro-2-biphenylyl)methyl]- 1,3,4-thiadiazol-2- yl}amino)carbonyl]-3,4-dihydro- 2(1H)-isoquinolinecarboxylate | 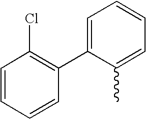 | 119 5-[(2′-chloro-2- biphenylyl)methyl]- 1,3,4-thiadiazol-2- amine | LC/MS: m/z 561.3 (M + H)⁺ Rt: 3.88 min. |
| 164 1,1-dimethylethyl 6-[({5-[(2- fluorophenyl)methyl]-1,3,4- thiadiazol-2-yl}amino)carbonyl]- 3,4-dihydro-2(1H)- isoquinolinecarboxylate | 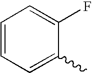 | Commercially available | LC/MS: m/z 469.08 (M + H)⁺ Rt: 3.56 min. |

The following Intermediates were prepared using the generic reaction scheme (Scheme 12)

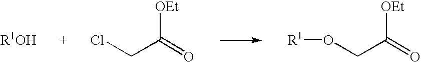

The following Intermediates were similarly prepared by a method analogous to that described for Intermediate 2.

TABLE 10

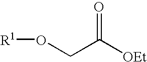

| Intermediate No. | R¹ | From: | Physical data |
|---|---|---|---|
| 165 Ethyl [(2-methylphenyl) oxy]acetate | 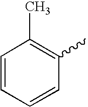 | Commercially available | ¹H NMR (300 MHz, DMSO, ppm) δ: 7.12 (m, 2H), 6.82 (m, 2H), 4.76 (s, 2H), 4.14 (q, 2H), 2.18 (s, 3H), 1.19 (t, 3H). |
| 166 Ethyl [(2,6- dichlorophenyl)oxy]acetate | 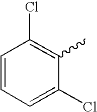 | Commercially available | ¹H NMR (300 MHz, DMSO, ppm) δ: 7.48 (d, 2H), 7.19 (t, 1H), 4.64 (s, 2H), 4.17 (q, 2H), 1.21 (t, 3H). |
| 167 Ethyl [(3,4- dimethylphenyl)oxy]acetate | 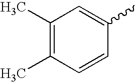 | Commercially available | ¹H NMR (300 MHz, DMSO, ppm) δ: 7.03 (d, 2H), 6.74 (d, 1H), 6.65 (dd, 1H), 4.58 (s, 2H), 4.27 (q, 2H), 2.23 (s, 3H), 2.20 (s, 3H), 1.30 (t, 3H). |
| 168 Ethyl {[2-chloro-3- (trifluoromethyl)phenyl] oxy}acetate | 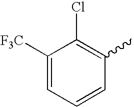 | Commercially available | LC/MS: m/z 283 (M + H)+ Rt: 2.02 min |
| 169 Ethyl [(2,4- difluorophenyl)oxy]acetate | 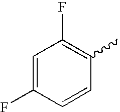 | Commercially available | LC/MS: m/z 217 (M + H)+ Rt: 1.85 min |

The following Intermediates were prepared using the generic reaction scheme (Scheme 12)

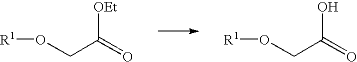

The following compounds were similarly prepared by a method analogous to that described for Intermediate 20.

TABLE 11

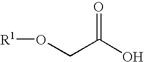

| | | From | |
|---|---|---|---|
| Intermediate No. | R¹ | Intermediate No. | Physical data |
| 170 [(2- methylphenyl)oxy] acetic acid | 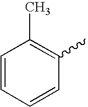 | 165 Ethyl [(2-methylphenyl) oxy]acetate | ¹H NMR (300 MHz, DMSO, ppm) δ: 7.11 (m, 2H), 6.79 (m, 2H), 4.66 (s, 2H), 2.16 (s, 3H). |
| 171 [(2,6-dichlorophenyl) oxy]acetic acid | 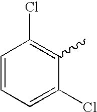 | 166 Ethyl [(2,6-dichlorophenyl) oxy]acetate | ¹H NMR (300 MHz, DMSO, ppm) δ: 7.49 (d, 2H), 7.19 (t, 1H), 4.55 (s, 2H). |
| 172 [(3,4-dimethylphenyl) oxy]acetic acid | 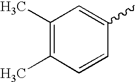 | 167 Ethyl [(3,4- dimethylphenyl)oxy] acetate | LC/MS: m/z 181.1 (M + H)+ Rt: 3.00 min |
| 173 {[2-chloro-3- (trifluoromethyl) phenyl]oxy}acetic acid | 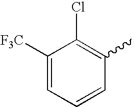 | 168 Ethyl {[2-chloro-3- (trifluoromethyl) phenyl]oxy}acetate | LC/MS: m/z 255 (M + H)+ Rt: 2.17 min |
| 174 [(2,4-difluorophenyl) oxy]acetic acid | 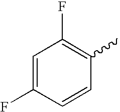 | 169 Ethyl [(2,4-difluorophenyl) oxy]acetate | ¹H NMR (300 MHz, DMSO, ppm) δ: 7.31 (m, 1H), 7.12 (m, 1H), 7.02 (m, 1H), 4.77 (s, 2H). |

The following Intermediates were prepared using the generic reaction scheme (Scheme 11)

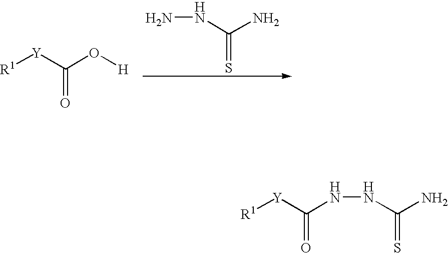

The following compounds Intermediates were similarly prepared by a method analogous method to that described for Intermediate 39.

TABLE 12

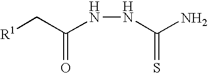

| Intermediate No. | R¹ | From: | Physical data |
|---|---|---|---|
| 175 2-[(2,4- dichlorophenyl)acetyl]hydrazinecarbothioamide | 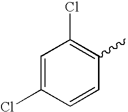 | Commercially available | LC/MS: m/z 279 (M + H)+ Rt: 2.21 min |
| 176 2-({2-[(trifluoromethyl)oxy] phenyl}acetyl) hydrazinecarbothioamide | 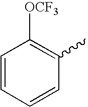 | Commercially available | LC/MS: m/z 294 (M + H)+ Rt: 2.17 min |
| 177 2-[(4-chloro-2- fluorophenyl)acetyl] hydrazinecarbothioamide | 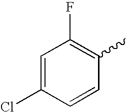 | Commercially available | LC/MS: m/z 262.0 (M + H)+ Rt: 2.02 min |
| 178 2-{[4-fluoro-2- (trifluoromethyl)phenyl] acetyl}hydrazine- carbothioamide | 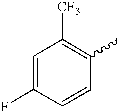 | Commercially available | LC/MS: m/z 296 (M + H)+ Rt: 2.15 min |
| 179 2-{[5-chloro-2- (trifluoromethyl)phenyl] acetyl}hydrazine- carbothioamide | 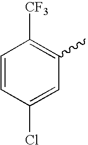 | Commercially available | LC/MS: m/z 313 (M + H)+ Rt: 2.34 min |

The following Intermediates were prepared using the generic reaction scheme (Scheme 11)

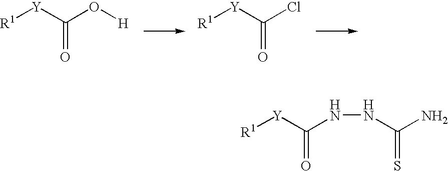

The following Intermediates were similarly prepared by a method analogous to that described for Intermediate 57.

TABLE 13

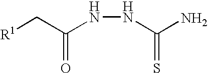

| | | | |
|---|---|---|---|
| | | From | |
| Intermediate No. | R¹ | Intermediate No. | Physical data |

| | | | |
|---|---|---|---|
| 180 2-{[(2-methylphenyl) oxy]acetyl} hydrazinecarbothioamide | 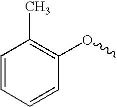 | [(2- methylphenyl) oxy]acetic acid 170 | ¹H NMR (300 MHz, DMSO, ppm) δ: 9.96 (bs, 1H), 9.26 (bs, 1H), 7.88 (bs, 1H), 7.50 (bs, 1H), 7.10 (m, 2H), 6.83 (m, 2H), 4.56 (s, 2H), 2.16 (s, 3H). |

The following Intermediates were prepared using the generic reaction scheme (Scheme 11)

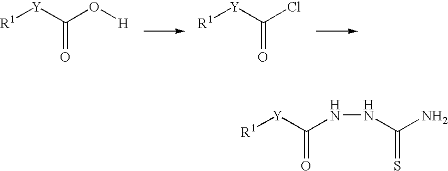

The following compounds were similarly prepared by a method analogous to that described for Intermediate 59.

TABLE 14

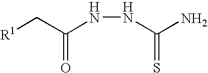

| | | From Intermediate | |
|---|---|---|---|
| Intermediate No. | R¹ | No. | Physical data |
| 181 2-[(3-chlorophenyl)acetyl] hydrazinecarbothioamide | 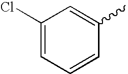 | Commercially available | 1H NMR (300 MHz, DMSO, ppm) δ: 10.1 (bs, 1H), 9.24 (bs, 1H), 7.90 (bs, 1H), 7.56 (bs, 1H), 7.32 (m, 4H), 3.52 (s, 2H). |
| 182 2-{[(2,6- dichlorophenyl)oxy] acetyl}hydrazine- carbothioamide | 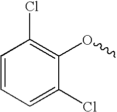 | 171 [(2,6- dichlorophenyl) oxy]acetic acid | LC/MS: m/z 295.1 (M + H)+ Rt: 1.50 min |
| 183 2-{[(3,4- dimethylphenyl)oxy] acetyl} hydrazinecarbothioamide | 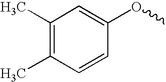 | 172 [(3,4- dimethylphenyl) oxy]acetic acid | 1H NMR (300 MHz, DMSO, ppm) δ: 10.02 (s, 1H), 9.25 (s, 1H), 7.87 (s, 1H), 7.48 (s, 1H), 6.99 (d, 1H), 6.66 (m, 2H), 4.47 (s, 1H), 2.13 (d, 6H). |
| 184 2-({[2-chloro-3- (trifluoromethyl)phenyl] oxy}acetyl) hydrazinecarbothioamide | 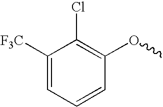 | 173 {[2-chloro-3- (trifluoromethyl) phenyl]oxy}acetic acid | LC/MS: m/z 328 (M + H)+ Rt: 1.57 min |
| 185 2-{[(2,4- difluorophenyl)oxy] acetyl}hydrazine- carbothioamide | 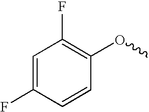 | 174 [(2,4- difluorophenyl)oxy] acetic acid | LC/MS: m/z 262 (M + H)+ Rt: 1.31 min |

The following Intermediates were prepared using the generic reaction scheme (Scheme 10)

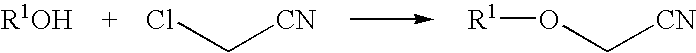

Intermediate 186: [(2,4,5-Trichlorophenyl)oxy]acetonitrile

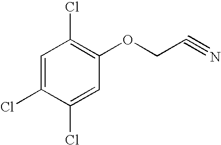

To a solution of 2,4,5-trichlorophenol (3 g, 15.2 mmol.) in acetone (50 mL) were added potassium carbonate (2.3 g, 16.7 mmol.) and then dropwise 2-chloroacetonitrile (1.26 g, 16.7 mmol.). The solution was refluxed overnight. After filtration, the filtrate was concentrated to dryness then poured into water (50 mL) and extracted with DCM (200 mL). The organic layer was washed with water then brine, dried on sodium sulphate and evaporated to dryness to give the title compound as dark solid (4.7 g, quantitative) which was used without further purification.

¹H NMR (300 MHz, DMSO, ppm) δ: 7.82 (s, 1H), 7.66 (s, 1H), 5.44 (s, 2H).

The following Intermediates were similarly prepared by a method analogous to that described for Intermediate 186

TABLE 15

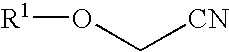

| Intermediate No. | R¹ | From: | Physical data |
|---|---|---|---|
| 187 {[2-chloro-5-(trifluoromethyl) phenyl]oxy}acetonitrile | 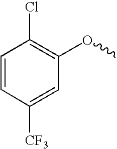 | Commercially available | ¹H NMR (300 MHz, CDCl3, ppm) δ: 7.55 (d, 1H), 7.28 (d, 1H), 7.26 (d, 1H), 4.89 (s, 2H). |
| 188 [(2-chloro-6-fluorophenyl) oxy]acetonitrile | 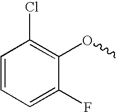 | Commercially available | ¹H NMR (300 MHz, CDCl3, ppm) δ: 7.12 (m, 1H), 7.08 (m, 2H), 4.86 (s, 2H). |
| 189 {[4-fluoro-2-(trifluoromethyl) phenyl]oxy}acetonitrile | 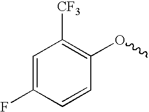 | Commercially available | ¹H NMR (300 MHz, CDCl3, ppm) δ: 7.37 (dd, 1H), 7.30 (m, 1H), 7.15 (m, 1H), 4.84 (s, 2H). |
| 190 {[2-chloro-4-(trifluoromethyl) phenyl]oxy}acetonitrile | 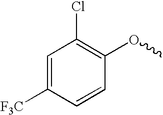 | Commercially available | ¹H NMR (300 MHz, CDCl3, ppm) δ: 7.71 (d, 1H), 7.57 (dd, 1H), 7.14 (d, 1H), 4.90 (s, 2H). |
| 191 [(3-chloro-5- fluorophenyl)oxy]acetonitrile | 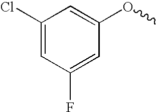 | Commercially available | ¹H NMR (300 MHz, CDCl3, ppm) δ: 6.85 (d, 1H), 6.79 (s, 1H), 6.63 (d, 1H), 4.75 (s, 2H). |
| 192 {[5-fluoro-2- (trifluoromethyl)phenyl] oxy}acetonitrile | 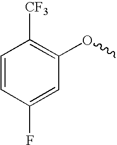 | Commercially available | ¹H NMR (300 MHz, CDCl3, ppm) δ: 7.65 (m, 1H), 6.87 (m, 2H), 4.86 (s, 2H). |
| 193 [(2-chloro-3,5- difluorophenyl) oxy]acetonitrile | 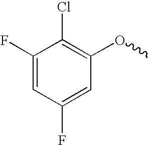 | Commercially available | ¹H NMR (300 MHz, DMSO, ppm) δ: 6.72 (td, 1H), 6.65 (dt, 1H), 4.85 (s, 2H). |

The following Intermediates were prepared using the generic reaction scheme (Scheme 10)

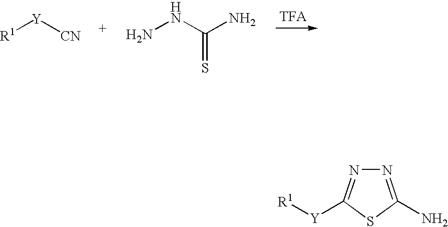

### Intermediate 194: 5-{[(2,4,5-Trichlorophenyl)oxy]methyl}-1,3,4-thiadiazol-2-amine

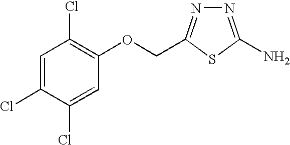

A mixture of [(2,4,5-trichlorophenyl)oxy]acetonitrile (Intermediate 186) (4.7 g, maximum 15.2 mmol.) and thiosemicarbazide (1.7 g, 18.6 mmol.) in trifluoroacetic acid (20 mL) was refluxed for 3 hours. Trifluoroacetic acid was removed under reduced pressure. To the residue was added 20 ml of cooled water and the mixture was adjusted to ph=6 to 7 with concentrated ammonia. The resulting solid material was filtered to give crude product which was stirred in methyl alcohol (20 mL) for 1.5 hours.

Then the solid material was filtered and dried to give the title compound as a white solid (4.45 g, 94%).

LC/MS: m/z 311.9 (M+H)⁺, Rt: 2.53 min.

The following Intermediates were similarly prepared by a method analogous to that described for Intermediate 194.

TABLE 16

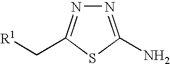

| | | From | |
|---|---|---|---|
| Intermediate No. | R¹ | Intermediate No. | Physical data |
| 195 5-({[2-chloro-5- (trifluoromethyl)phenyl]oxy} methyl)-1,3,4-thiadiazol-2- amine | 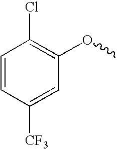 | 187 {[2-chloro-5- (trifluoromethyl) phenyl]oxy}acetonitrile | LC/MS: m/z 310.0 (M + H)⁺, Rt: 2.44 min. |
| 196 5-({[2-chloro-5- (trifluoromethyl)phenyl]oxy} methyl)-1,3,4-thiadiazol-2- amine | 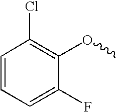 | 188 [(2-chloro-6- fluorophenyl)oxy] acetonitrile | LC/MS: m/z 260.0 (M + H)⁺, Rt: 2.12 min. |
| 197 5-({[4-fluoro-2- (trifluoromethyl)phenyl]oxy} methyl)-1,3,4-thiadiazol-2- amine | 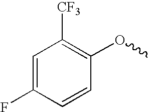 | 189 {[4-fluoro-2- (trifluoromethyl) phenyl]oxy}acetonitrile | LC/MS: m/z 294.0 (M + H)⁺, Rt: 2.33 min. |
| 198 5-({[2-chloro-4- (trifluoromethyl)phenyl]oxy} methyl)-1,3,4-thiadiazol-2- amine | 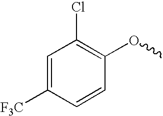 | 190 {[2-chloro-4- (trifluoromethyl) phenyl]oxy}acetonitrile | LC/MS: m/z 310.0 (M + H)⁺, Rt: 2.46 min. |
| 199 5-{[(3-chloro-5- fluorophenyl)oxy]methyl}- 1,3,4-thiadiazol-2-amine | 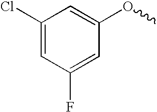 | 191 [(3-chloro-5-fluorophenyl) oxy]acetonitrile | LC/MS: m/z 260.0 (M + H)⁺, Rt: 2.29 min. |
| 200 5-{[(3-chloro-5- fluorophenyl)oxy]methyl}- 1,3,4-thiadiazol-2-amine | 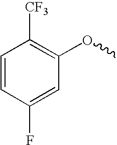 | 192 {[5-fluoro-2- (trifluoromethyl) phenyl]oxy}acetonitrile | LC/MS: m/z 294.0 (M + H)⁺, Rt: 2.31 min. |
| 201 5-{[(2-chloro-3,5- difluorophenyl)oxy]methyl}- 1,3,4-thiadiazol-2-amine | 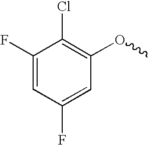 | 193 [(2-chloro-3,5- difluorophenyl) oxy]acetonitrile | LC/MS: m/z 278.0 (M + H)⁺, Rt: 2.29 min. |

The following Intermediates were prepared using the generic reaction scheme (Scheme 8)

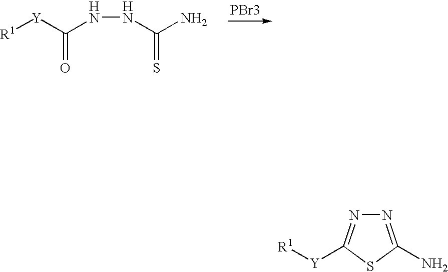

The following Intermediates were similarly prepared by a method analogous to that described for Intermediate 77.

TABLE 17

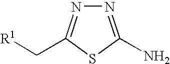

| Intermediate No. | R¹ | From Intermediate No. | Physical data |
|---|---|---|---|
| 202 5-[(2- chlorophenyl)methyl]- 1,3,4-thiadiazol-2- amine | 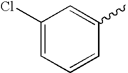 | 181 2-[(3-chlorophenyl)acetyl] hydrazinecarbothioamide | LC/MS: m/z 226.0 (M + H )+, Rt: 3.42 min. |
| 203 5-{[(2,6- dichlorophenyl)oxy] methyl}-1,3,4- thiadiazol-2-amine | 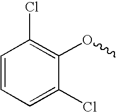 | 182 2-{[(2,6- dichlorophenyl)oxy] acetyl}hydrazine- carbothioamide | LC/MS: m/z 277 (M + H)+, Rt: 2.33 min. |
| 204 5-{[(2- methylphenyl)oxy] methyl}-1,3,4- thiadiazol-2-amine | 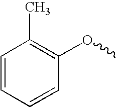 | 180 2-{[(2- methylphenyl)oxy]acetyl} hydrazinecarbothioamide | LC/MS: m/z 222.0 (M + H)+, Rt: 3.71 min. |
| 205 5-{[(3,4- dimethylphenyl)oxy] methyl}-1,3,4- thiadiazol-2-amine | 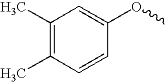 | 183 2-{[(3,4- dimethylphenyl)oxy] acetyl}hydrazine- carbothioamide | LC/MS: m/z 236.0 (M + H)+, Rt: 2.33 min. |
| 206 5-({[2-chloro-3- (trifluoromethyl)phenyl] oxy}methyl)-1,3,4- thiadiazol-2-amine | 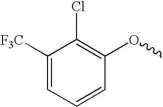 | 184 2-({[2-chloro-3- (trifluoromethyl)phenyl] oxy}acetyl) hydrazinecarbothioamide | LC/MS: m/z 310 (M + H)+, Rt: 2.43 min. |
| 207 5-{[(2,4- difluorophenyl)oxy] methyl}-1,3,4- thiadiazol-2-amine | 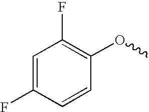 | 185 2-{[(2,4- difluorophenyl)oxy] acetyl}hydrazine- carbothioamide | LC/MS: m/z 244 (M + H)+, Rt: 2.06 min. |

The following Intermediates were prepared using the generic reaction scheme (Scheme 8)

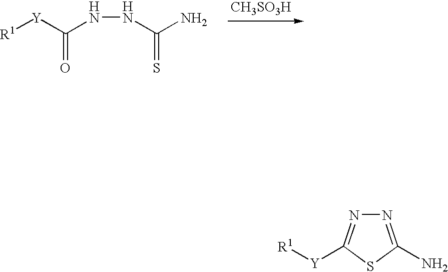

The following Intermediates were similarly prepared by analogous method to that described for Intermediate 95.

TABLE 18

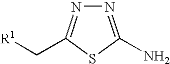

| Intermediate No. | R¹ | From Intermediate No. | Physical data |
|---|---|---|---|
| 208 5-[(2,4- dichlorophenyl)meth- yl]-1,3,4-thiadiazol-2- amine | 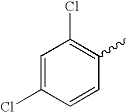 | 175 2-[(2,4- dichlorophenyl)acetyl] hydrazinecarbothioamide | LC/MS: m/z 260 (M + H)⁺ Rt: 2.61 min |
| 209 5-({2- [(trifluoromethyl)oxy] phenyl}methyl)-1,3,4- thiadiazol-2-amine | 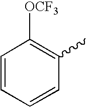 | 176 2-({2- [(trifluoromethyl)oxy] phenyl}acetyl)hydrazine- carbothioamide | ¹H NMR (300 MHz, DMSO, ppm) δ: 7.41 (m, 4H), (bs, 2H), 4.20 (s, 2H). |
| 210 5-[(4-chloro-2- fluorophenyl)methyl]- 1,3,4-thiadiazol-2- amine | 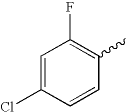 | 177 2-[(4-chloro-2- fluorophenyl)acetyl] hydrazinecarbothioamide | LC/MS: m/z 244.0 (M + H)⁺ Rt: 2.47 min |
| 211 5-{[4-fluoro-2- (trifluoromethyl)phen- yl]methyl}-1,3,4- thiadiazol-2-amine | 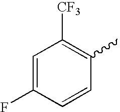 | 178 2-{[4-fluoro-2- (trifluoromethyl)phenyl] acetyl}hydrazine- carbothioamide | LC/MS: m/z 278 (M + H)⁺, Rt: 2.58 min. |
| 212 5-{[5-chloro-2- (trifluoromethyl)phen- yl]methyl}-1,3,4- thiadiazol-2-amine | 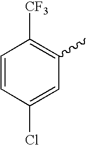 | 179 2-{[5-chloro-2- (trifluoromethyl)phenyl] acetyl}hydrazine- carbothioamide | LC/MS: m/z 294 (M + H)⁺, Rt: 2.73 min. |

The following Intermediates were prepared using the generic reaction scheme (Scheme 1):

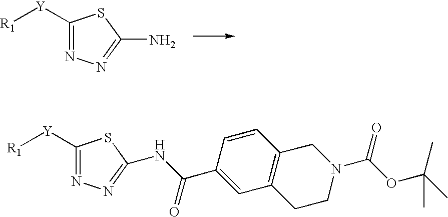

The following Intermediates were similarly prepared by a method analogous to that described for Intermediate 120.

TABLE 19

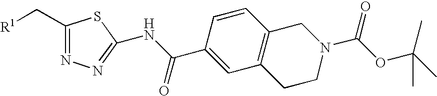

| | | From | |
|---|---|---|---|
| Intermediate No. | R¹ | Intermediate No. | Physical data |
| 213 1,1-dimethylethyl 6-[({5- [(3-chlorophenyl)methyl]- 1,3,4-thiadiazol-2- yl}amino)carbonyl]-3,4- dihydro-2(1H)- isoquinolinecarboxylate | 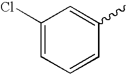 | 202 5-[(2- chlorophenyl)meth- yl]-1,3,4- thiadiazol-2-amine | LC/MS: m/z 485.0 (M + H)+ Rt: 3.59 min. |
| 214 1,1-dimethylethyl 6-{[(5- {[(2,6- dichlorophenyl)oxy]methyl}- 1,3,4-thiadiazol-2- yl)amino]carbonyl}-3,4- dihydro-2(1H)- isoquinolinecarboxylate | 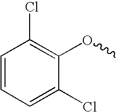 | 203 5-{[(2,6- dichlorophenyl)oxy] methyl}-1,3,4- thiadazol-2-amine | LC/MS: m/z 535.06 (M + H)+ Rt: 3.88 min. |
| 215 1,1-dimethylethyl 6-{[(5- {[(2- methylphenyl)oxy]methyl}- 1,3,4-thiadiazol-2- yl)amino]carbonyl}-3,4- dihydro-2(1H)- isoquinolinecarboxylate | 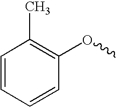 | 204 5-{[(2- methylphenyl)oxy] methyl}-1,3,4- thiadiazol-2-amine | ¹H NMR (400 MHz, DMSO, ppm) δ: 13.06 (bs, 1H), 7.95 (m, 2H), 7.39 (d, 1H), 7.18 (m, 3H), 6.93 (m, 1H), 5.55 (s, 2H), 4.61 (bs, 2H), 3.61 (m, 2H), 2.88 (m, 2H), 2.23 (s, 3H), 1.45 (s, 9H). |
| 216 1,1-dimethylethyl 6-{[(5- {[(3,4- dimethylphenyl)oxy]methyl}- 1,3,4-thiadiazol-2- yl)amino]carbonyl}-3,4- dihydro-2(1H)- isoquinolinecarboxylate | 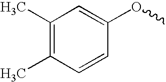 | 205 5-{[(3,4- dimethylphenyl) oxy]methyl}-1,3,4- thiadiazol-2-amine | LC/MS: m/z 494.8 (M + H)+ Rt: 3.82 min. |
| 217 1,1-dimethylethyl 6-[({5- [(2,4- dichlorophenyl)methyl]- 1,3,4-thiadiazol-2- yl}amino)carbonyl]-3,4- dihydro-2(1H)- isoquinolinecarboxylate | 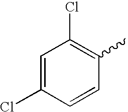 | 208 5-[(2,4- dichlorophenyl) methyl]-1,3,4- thiadiazol-2-amine | LC/MS: m/z 519 (M + H)+ Rt: 4.30 min. |
| 218 1,1-dimethylethyl 6-({[5- ({2- [(trifluoromethyl)oxy]phen- yl}methyl)-1,3,4-thiadiazol- 2-yl]amino}carbonyl)-3,4- dihydro-2(1H)- isoquinolinecarboxylate | 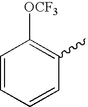 | 209 5-({2- [(trifluoromethyl) oxy]phenyl}methyl)- 1,3,4-thiadiazol-2- amine | LC/MS: m/z 535.1 (M + H)+ Rt: 3.74 min. |
| 219 1,1-dimethylethyl 6-{[(5- {[(2-chloro-3,5- difluorophenyl)oxy]methyl}- 1,3,4-thiadiazol-2- yl)amino]carbonyl}-3,4- dihydro-2(1H)- isoquinolinecarboxylate | 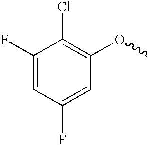 | 201 5-{[(2-chloro-3,5- difluorophenyl)oxy] methyl}-1,3,4- thiadiazol-2-amine | LC/MS: m/z 536.0 (M + H)+ Rt: 3.67 min |
| 220 1,1-dimethylethyl 6-{[(5- {[(2-chloro-6- fluorophenyl)oxy]methyl}- 1,3,4-thiadiazol-2- yl)amino]carbonyl}-3,4- dihydro-2(1H)- isoquinolinecarboxylate | 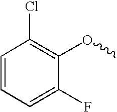 | 196 5-({[2-chloro-5- (trifluoromethyl) phenyl]oxy}methyl)- 1,3,4-thiadiazol-2- amine | ¹H NMR (400 MHz, DMSO, ppm) δ: 13.06 (bs, 1H), 7.95 (m, 2H), 7.35 (m, 3H), 7.23 (m, 1H), 5.53 (s, 2H), 4.60 (bs, 2H), 3.60 (t, 2H), 2.88 (t, 2H), 1.45 (s, 9H). |
| 221 1,1-dimethylethyl 6-({[5- ({[2-chloro-3- (trifluoromethyl)phenyl]oxy} methyl)-1,3,4-thiadiazol- 2-yl]amino}carbonyl)-3,4- dihydro-2(1H)- isoquinolinecarboxylate | 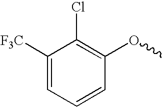 | 206 5-({[2-chloro-3- (trifluoromethyl) phenyl]oxy}methyl)- 1,3,4-thiadiazol-2- amine | LC/MS: m/z 568.8 (M + H)+ Rt: 3.86 min. |
| 222 1,1-dimethylethyl 6-{[(5- {[(2,4,5- trichlorophenyl)oxy]methyl}- 1,3,4-thiadiazol-2- yl)amino]carbonyl}-3,4- dihydro-2(1H)- isoquinolinecarboxylate | 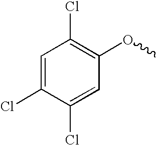 | 194 5-{[(2,4,5- trichlorophenyl) oxy]methyl}-1,3,4- thiadiazol-2-amine | LC/MS: m/z 568.7 (M + H)+ Rt: 4.03 min. |
| 223 1,1-dimethylethyl 6-({[5- ({[2-chloro-5- (trifluoromethyl)phenyl]oxy} methyl)-1,3,4-thiadiazol- 2-yl]amino}carbonyl)-3,4- dihydro-2(1H)- isoquinolinecarboxylate | 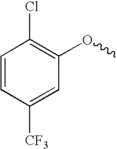 | 195 5-({[2-chloro-5- (trifluoromethyl) phenyl]oxy}methyl)- 1,3,4-thiadiazol-2- amine | LC/MS: m/z 569.0 (M + H)+ Rt: 3.91 min. |
| 224 1,1-dimethylethyl 6-({[5- ({[2-chloro-5- (trifluoromethyl)phenyl]oxy} methyl)-1,3,4-thiadiazol- 2-yl]amino}carbonyl)-3,4- dihydro-2(1H)- isoquinolinecarboxylate | 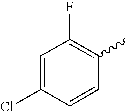 | 210 5-[(4-chloro-2- fluorophenyl)meth- yl]-1,3,4- thiadiazol-2-amine | LC/MS: m/z 503.08 (M + H)+ Rt: 3.78 min. |
| 225 1,1-dimethylethyl 6-{[(5- {[4-fluoro-2- (trifluoromethyl)phenyl]meth- yl}-1,3,4-thiadiazol-2- yl)amino]carbonyl}-3,4- dihydro-2(1H)- isoquinolinecarboxylate | 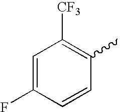 | 211 5-{[4-fluoro-2- (trifluoromethyl) phenyl]methyl}-1,3,4- thiadiazol-2-amine | LC/MS: m/z 537.0 (M + H)+ Rt: 3.74 min. |
| 226 1,1-dimethylethyl 6-{[(5- {[5-chloro-2- (trifluoromethyl)phenyl]meth- yl}-1,3,4-thiadiazol-2- yl)amino]carbonyl}-3,4- dihydro-2(1H)- isoquinolinecarboxylate | 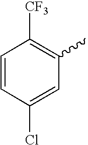 | 212 5-{[5-chloro-2- (trifluoromethyl) phenyl]methyl}-1,3,4- thiadiazol-2-amine | LC/MS: m/z 553.0 (M + H)+ Rt: 3.85 min. |
| 227 1,1-dimethylethyl 6-({[5- ({[4-fluoro-2- (trifluoromethyl)phenyl]oxy} methyl)-1,3,4-thiadiazol- 2-yl]amino}carbonyl)-3,4- dihydro-2(1H)- isoquinolinecarboxylate | 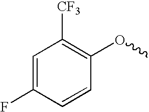 | 197 5-({[4-fluoro-2- (trifluoromethyl) phenyl]oxy}methyl)- 1,3,4-thiadiazol-2- amine | LC/MS: m/z 553.1 (M + H)+ Rt: 3.85 min. |
| 228 1,1-dimethylethyl 6-({[5- ({[2-chloro-4- (trifluoromethyl)phenyl]oxy} methyl)-1,3,4-thiadiazol- 2-yl]amino}carbonyl)-3,4- dihydro-2(1H)- isoquinolinecarboxylate | 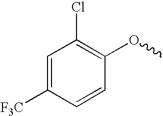 | 198 5-({[2-chloro-4- (trifluoromethyl) phenyl]oxy}methyl)- 1,3,4-thiadiazol-2- amine | LC/MS: m/z 569.1 (M + H)+ Rt: 4.02 min. |
| 229 1,1-dimethylethyl 6-{[(5- {[(3-chloro-5- fluorophenyl)oxy]methyl}- 1,3,4-thiadiazol-2- yl)amino]carbonyl}-3,4- dihydro-2(1H)- isoquinolinecarboxylate | 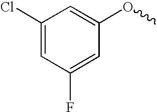 | 199 5-{[(3-chloro-5- fluorophenyl)oxy] methyl}-1,3,4- thiadiazol-2-amine | LC/MS: m/z 518.7 (M + H)+ Rt: 3.81 min. |
| 230 1,1-dimethylethyl 6-({[5- ({[5-fluoro-2- (trifluoromethyl)phenyl]oxy} methyl)-1,3,4-thiadiazol- 2-yl]amino}carbonyl)-3,4- dihydro-2(1H)- isoquinolinecarboxylate | 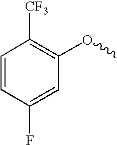 | 200 5-{[(3-chloro-5- fluorophenyl)oxy] methyl}-1,3,4- thiadiazol-2-amine | LC/MS: m/z 552.8 (M + H)+ Rt: 3.74 min. |
| 231 1,1-dimethylethyl 6-{[(5- {[(2,4- difluorophenyl)oxy]methyl}- 1,3,4-thiadiazol-2- yl)amino]carbonyl}-3,4- dihydro-2(1H)- isoquinolinecarboxylate | 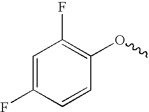 | 207 5-{[(2,4- difluorophenyl)oxy] methyl}-1,3,4- thiadiazol-2-amine | 1H NMR (400 MHz, DMSO, ppm) δ: 13.05 (bs, 1H), 7.95 (m, 2H), 7.38 (m, 3H), 7.07 (t, 1H), 5.59 (s, 2H), 4.59 (bs, 2H), 3.60 (t, 2H), 2.87 (t, 2H), 1.44 (s, 9H). |

The following Intermediates were prepared using the generic reaction scheme (Scheme 19)

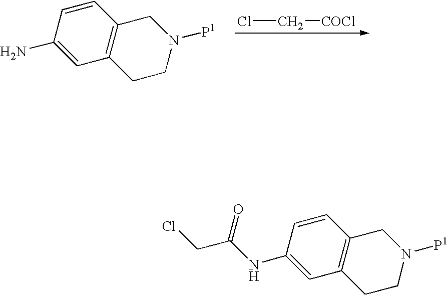

### Intermediate 232: 1,1-Dimethylethyl 6-[(chloroacetyl)amino]-3,4-dihydro-2(1H)-isoquinolinecarboxylate

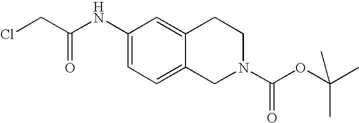

To a solution of 1,1-dimethylethyl 6-amino-3,4-dihydro-2(1H)-isoquinolinecarboxylate (9.5 g, 38.3 mmol.) in THF (350 mL) under nitrogen and cooled to 0° C. were added sodium hydrogenocarbonate (8 g, 95.6 mmol.) and after 2 to 3 minutes of stirring, drop-wise, a solution of chloroacetyl chloride (6.1 ml, 76.5 mmol.) in THF (10 mL). The mixture was stirred at 0° C. for 10 minutes then heated up to room temperature and stirred for 2.5 hours. The mixture was poured into an aqueous saturated solution of sodium hydrogenocarbonate and ethyl acetate (500 ml) was added. The organic layer was washed three times with aqueous saturated solution of sodium hydrogenocarbonate then dried on sodium sulphate, filtered and evaporated to dryness to give the title compound as yellow oil which crystallised slowly (14.09 g, quantitative yield).

¹H NMR (400 MHz, DMSO, ppm) δ: 10.2 (bs, 1H), 7.44 (bs, 1H), 7.36 (bd, 1H), 7.12 (d, 1H), 4.45 (m, 2H), 4.23 (s, 2H), 3.54 (t, 2H), 2.75 (t, 2H), 1.43 (s, 9H).

The following Intermediates were prepared using the generic reaction scheme (Scheme 18)

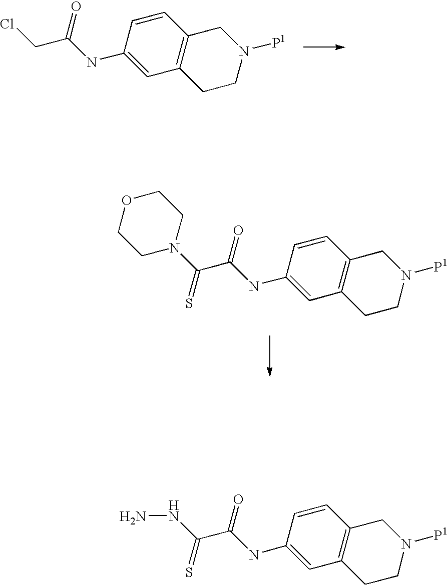

### Intermediate 233: 1,1-Dimethylethyl 6-{[4-morpholinyl(thioxo)acetyl]amino}-3,4-dihydro-2(1H)-isoquinolinecarboxylate

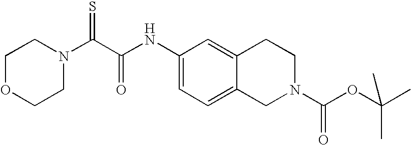

To a solution of morpholine (4.6 mL, 52 mmol.) in DMF (30 mL) were added at room temperature sulphur S₈ (4.2 g, 130 mmol) and then a solution of 1,1-dimethylethyl 6-[(chloroacetyl)amino]-3,4-dihydro-2(1H)-isoquinolinecarboxylate (Intermediate 232) (12.43 g, maximum 38.3 mmol) in DMF (170 mL). After stirring for 24 hours, water was added. The solid material was difficult to filter and the partially filtered material was poured in acetone. The remaining solid material was removed by filtration. The two filtrates were diluted with DCM then dried on sodium sulphate, filtered and evaporated to dryness. The residue was purified by flash column chromatography eluting with a gradient cyclohexane 100% to cyclohexane/EtOAc:40/60 to give the title compound as a yellow oil (3.05 g, 19.6%).

¹H NMR (400 MHz, DMSO, ppm) δ: 10.56 (bs, 1H), 7.48 (bs, 1H), 7.38 (bd, 1H), 7.14 (d, 1H), 4.45 (m, 2H), 4.12 (t, 2H), 3.76 (t, 2H), 3.68 (bs, 4H), 3.53 (t, 2H), 2.76 (t, 2H), 1.43 (s, 9H).

### Intermediate 234: 1,1-Dimethylethyl 6-{[hydrazino(thioxo)acetyl]amino}-3,4-dihydro-2(1H)-isoquinolinecarboxylate

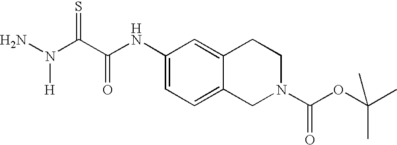

To a solution of 1,1-dimethylethyl 6-{[4-morpholinyl(thioxo)acetyl]amino}-3,4-dihydro-2(1H)-isoquinolinecarboxylate (Intermediate 233) (3.05 g, 7.5 mmol.) in DMF (25 mL) was added hydrazine hydrate (5 mL, 103 mmol). After stirring overnight at room temperature, water was added and pH was adjusted to pH=4 to 5 with concentrated hydrochloride solution. The solid material was filtered and washed twice with a minimal amount of water. The residue was refluxed in ethyl alcohol. After return to room temperature, the solid was filtered and washed with ethyl alcohol to give after drying the title compound as a beige solid (1.64 g, 63%).

¹H NMR (400 MHz, DMSO, ppm) δ: 10.14 (bs, 1H), 7.56 (m, 2H), 7.16 (d, 1H), 4.47 (bs, 2H), 3.55 (t, 2H), 2.77 (t, 2H), 1.43 (s, 9H).

The following Intermediates were prepared using the generic reaction scheme (Scheme 17)

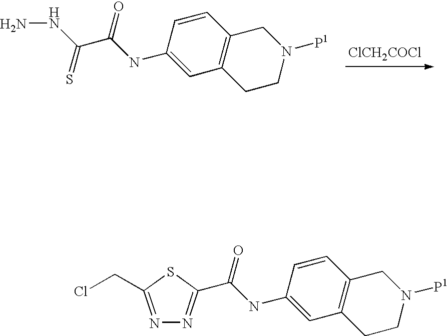

### Intermediate 235: 1,1-Dimethylethyl 6-({[5-(chloromethyl)-1,3,4-thiadiazol-2-yl]carbonyl}amino)-3,4-dihydro-2(1H)-isoquinolinecarboxylate

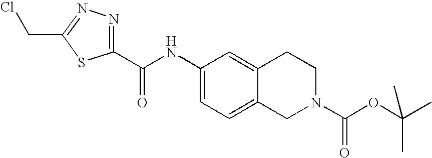

To a solution of 1,1-dimethylethyl 6-{[hydrazino(thioxo)acetyl]amino}-3,4-dihydro-2(1H)-isoquinolinecarboxylate (Intermediate 234) (1 g, 2.85 mmol.) in DMF (40 mL) was added under nitrogen and dropwise a solution of chloroacetyl chloride (2.3 ml, 28.5 mmol.) in a small volume of DMF. After stirring overnight at room temperature, water was added. The pasty solid was isolated from the liquid layer, dissolved in DCM and evaporated to dryness. The residue was purified by flash column chromatography eluting with a gradient DCM 100% to DCM/MeOH:96/4 to give the title compound as light yellow oil which crystallized slowly (232 mg g, 20%).

¹H NMR (400 MHz, DMSO, ppm) δ: 11.15 (s, 1H), 7.69 (s, 1H), 7.62 (d, 1H), 7.18 (d, 1H), 5.34 (s, 2H), 4.48 (bs, 2H), 3.56 (t, 2H), 2.78 (t, 2H), 1.44 (s, 9H).

The following Intermediates were prepared using the generic reaction scheme (Scheme 15)

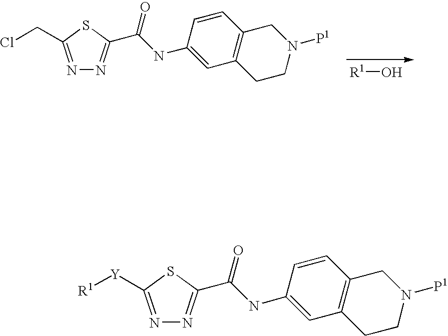

### Intermediate 236: 1,1-Dimethylethyl 6-{[(5-{[(2-chlorophenyl)oxy]methyl}-1,3,4-thiadiazol-2-yl)carbonyl]amino}-3,4-dihydro-2(1H)-isoquinolinecarboxylate

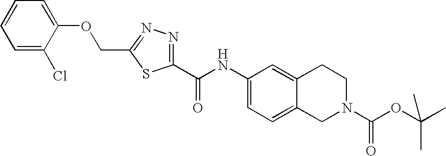

To a solution of 2-chlorophenol (60 mg, 0.47 mmol.) in acetone (10 mL) was added potassium carbonate (102 mg, 0.74 mmol) then after 1 hour of stirring at room temperature 1,1-dimethylethyl 6-({[5-(chloromethyl)-1,3,4-thiadiazol-2-yl]carbonyl}amino)-3,4-dihydro-2(1H)-isoquinolinecarboxylate (Intermediate 235) (232 mg, 0.57 mmol). The mixture was refluxed for 5 hours then an addition amount of 2-chlorophenol (12 mg, 0.1 mmol) was added and the mixture was heated overnight at 45° C. After return to room temperature, the solid material was removed by filtration and the filtrate was evaporated to dryness. The crude material was poured in DCM and water was added. The aqueous layer was extracted with DCM and the combined organic layer was washed with brine then dried on sodium sulphate. After filtration and evaporation to dryness, the residue was purified by flash column chromatography eluting with a gradient DCM 100% to DCM/MeOH:96/4. After evaporation of the right fractions, the material was crystallized with a small volume of diisopropyl ether to give after drying the title compound as an off white solid (140 mg, 49%).

¹H NMR (400 MHz, DMSO, ppm) δ: 11.14 (s, 1H), 7.69 (bs, 1H), 7.63 (d, 1H), 7.51 (d, 1H), 7.36 (d, 2H), 7.18 (d, 1H), 7.07 (m, 1H), 5.79 (s, 2H), 4.48 (bs, 2H), 3.56 (t, 2H), 2.78 (t, 2H), 1.44 (s, 9H).

### Intermediate 237: 2-Chloro-N-methylaniline

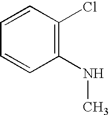

To a solution of 2-chloro aniline (26 g, 0.204 mol.) in THF (260 mL) was added butyl lithium (2.5 M, 80 mL, 0.2 mol) at −50° C. The reaction was stirred at −50° C. for 0.5 hours then allowed to reach room temperature. After 0.5 hours at room temperature, the mixture was cooled to −50° C. then iodomethane (12.4 mL, 0.2 mol.) was added. After stirring at −50° C. for 0.5 hours, the mixture was warmed up to room temperature and stirred for 5 hours. The mixture was poured in saturated NH₄Cl solution then the aqueous layer was extracted with diethyl ether. The combined organic layers were washed with water then dried on sodium sulphate. After filtration and evaporation to dryness, the residue was purified by flash column chromatography eluting with EtOAc/petroleum ether: 1/100 to give the title compound as a clear oil (12 g, 42%).

¹H NMR (300 MHz, DMSO, ppm) δ: 7.21 (d, 1H), 7.13 (t, 1H), 6.50 (m, 2H), 5.45 (bs, 1H), 2.72 (d, 3H).

### Intermediate 238: N-(2-Chlorophenyl)-N-methylglycine

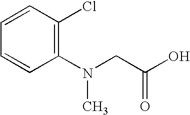

To a solution of the 2-chloro-N-methylaniline (Intermediate 237) (8 g, 0.057 mol.) in acetonitrile (340 mL) was added oxoacetic acid (42.2 g, 0.57 mol.). After stirring for 0.5 hours at room temperature, NaBH₃CN (17.7 g, 0.285 mol) was added in portions by keeping the temperature below 40° C. The mixture was stirred at room temperature for 2 hours then acetic acid (23 mL) was added drop-wise. After stirring for 1 hour, the solid material was removed by filtration and the filtrate was concentrated to dryness. The residue was poured in water and the pH was adjusted to 9 with aqueous NaOH. After extraction of the aqueous layer with EtOAc, the aqueous layer was acidified to pH: 4 with diluted HCl. The white solid was filtered, washed with water then dried to give the title compound as a white solid (7 g, 62%).

LC/MS: m/z 200.1 (M+H)⁺, Rt: 1.67 min.

### Intermediate 239: 2-{[(2-Chlorophenyl)(methyl)amino]acetyl}hydrazinecarbothioamide

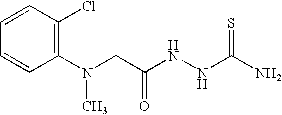

To a solution of N-(2-chlorophenyl)-N-methylglycine (Intermediate 238) (3 g, 0.015 mol.) in dichloromethane containing 3 drops of DMF, was slowly added oxalyl chloride (2.3 g, 0.018 mol.). After stirring for 2 hours at room temperature, the solvent was evaporated under vacuum. The residue was dissolved in DMF (10 mL) then added to a solution of thiosemicarbazide (1.45 g, 0.016 mol.) and pyridine (1.26 g, 0.016 mol.) in DMF (30 mL). After stirring for 2 hours at room temperature, the mixture was poured into water (500 mL) and stirred for several hours. The resulting solid material was filtered, washed with EtOAc and dried to give the title compound as a white solid (3 g, 69%).

LC/MS: m/z 273.0 (M+H)⁺, Rt: 2.07 min.

### Intermediate 240: 5-{[(2-Chlorophenyl)(methyl)amino]methyl}-1,3,4-thiadiazol-2-amine

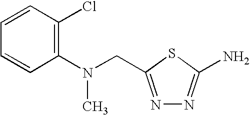

To a solution of 2-{[(2-chlorophenyl)(methyl)amino]acetyl}hydrazinecarbothioamide (Intermediate 239) (3 g, 11 mmol) in toluene (20 ml) was added drop by drop methane sulphonic acid (9 mL, 138 mmol) and the reaction mixture was refluxed for 3 hours. The solvent was evaporated. The residue was diluted with water and ammonia solution was added until pH=8. The solid material was filtered, washed with water and EtOAc, and dried to give the title compound as a white solid (2 g, 71%).

LC/MS: m/z 255.1 (M+H)⁺, Rt: 2.17 min.

### Intermediate 241: 1,1-Dimethylethyl 6-{[(5-{[(2-chlorophenyl)(methyl)amino]methyl}-1,3,4-thiadiazol-2-yl)amino]carbonyl}-3,4-dihydro-2(1H)-isoquinolinecarboxylate

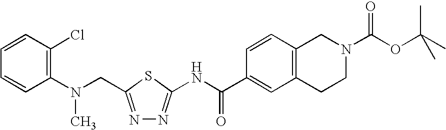

A solution of 2-{[(1,1-dimethylethypoxy]carbonyl}-1,2,3,4-tetrahydro-6-isoquinolinecarboxylic acid (555 mg, 2 mmol), HATU (989 mg, 2.6 mmol), triethylamine (0.26 mL, 2.6 mmol) in DMF (15 mL) and 5-{[(2-chlorophenyl)(methyl)amino]methyl}-1,3,4-thiadiazol-2-amine (Intermediate 240) (509 mg, 2 mmol) was stirred at room temperature overnight. The DMF was evaporated under reduced pressure and the residue was dissolved in DCM. The organic phase was dried over sodium sulphate, filtered and evaporated under reduced pressure. The residue was purified by flash column chromatography eluting with a gradient DCM 100% to DCM/MeOH:98/2. After evaporation of the right fractions, the material was triturated in hot methyl alcohol, to give after filtration and drying the title compound as a white solid (300 mg, 29%).

HRMS calculated for C₂₅H₂₈ClN₅O₃S (M+H)⁺ 514.1680, found: 514.1651, Rt: 3.47 min.

The following Intermediates were prepared using the generic reaction scheme (Scheme 6)

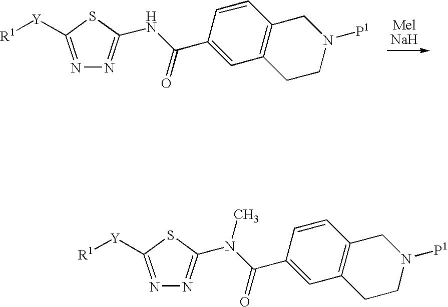

### Intermediate 242: 1,1-Dimethylethyl 6-{[(5-{[(2-chlorophenyl)oxy]methyl}-1,3,4-thiadiazol-2-yl)(methyl)amino]carbonyl}-3,4-dihydro-2(1H)-isoquinolinecarboxylate

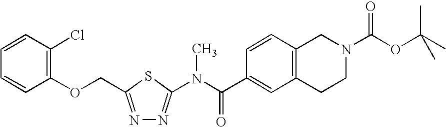

To a solution of 5-[(2′-chloro-2-biphenylyl)methyl]-1,3,4-thiadiazol-2-amine 1,1-dimethylethyl 6-{[(5-{[(2-chlorophenyl)oxy]methyl}-1,3,4-thiadiazol-2-yl)amino]carbonyl}-3,4-dihydro-2(1H)-isoquinolinecarboxylate (Intermediate 120) (180 mg, 0.36 mmol.) in THF (10 mL) was added NaH 60% in mineral oil (15 mg, 0.378 mmol) then after 1 hour of stirring at room temperature iodomethane (53 mg, 0.378 mmol). The mixture was stirred for 2 days at room temperature. After evaporation to dryness, the crude material was poured in DCM and washed with water. After filtration and evaporation to dryness, the residue was triturated with diisopropyl ether to give after drying the title compound as a light yellow solid (150 mg, 81%).

LC/MS: m/z 515 (M+H)⁺, Rt: 4.18 min.

### Intermediate 243: 1,1-Dimethylethyl 6-[({5-[(3,4-dichlorophenyl)methyl]-1,3,4-thiadiazol-2-yl}carbonyl)amino]-3,4-dihydro-2(1H)-isoquinolinecarboxylate

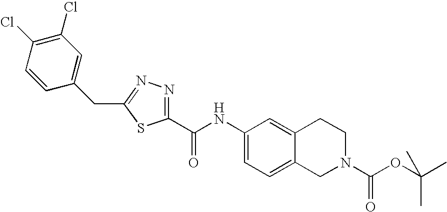

A solution of (3,4-dichlorophenyl)acetic acid (320 mg, 1.56 mmol), HATU (890 mg, 2.34 mmol), DIPEA (270 μL, 2.34 mmol) in DMF (15 mL) was stirred at room temperature for 1 hour. 1,1-dimethylethyl 6-{[hydrazino(thioxo)acetyl]amino}-3,4-dihydro-2(1H)-isoquinolinecarboxylate, (Intermediate 234) (600 mg, 1.71 mmol) was added and the mixture was stirred at room temperature for 10 days. The DMF was evaporated under reduced pressure and the residue was dissolved in dichloromethane. The organic phase was then washed with water. The aqueous phase was extracted with dichloromethane. Then the combined organic phase was treated with brine, dried over sodium sulfate, filtered and evaporated under reduced pressure. The residue was purified by flash column chromatography eluting with a gradient DCM 100% to DCM/MeOH:96/4 to give the title compound as yellow oil (200 mg, 24%).

LC/MS:m/z: 518.9 (M+H)⁺, Rt: 3.96 min.

The following Examples were prepared using the generic reaction scheme (Scheme 1):

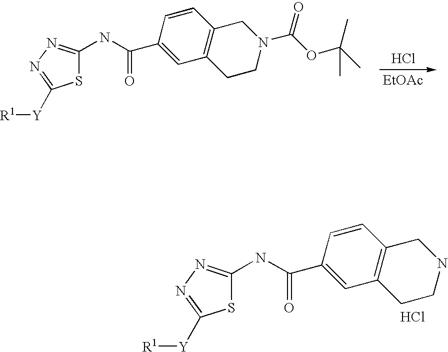

### EXAMPLE 1

### N-(5-{[(2-Chlorophenyl)oxy]methyl}-1,3,4-thiadiazol-2-yl)-1,2,3,4-tetrahydro-6-isoquinolinecarboxamide hydrochloride

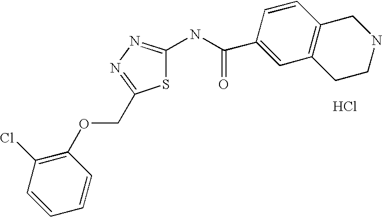

HCl(g) was bubbled at 0° C. in EtOAc until the solvent was saturated and 1,1-dimethylethyl 6-{[(5-{[(2-chlorophenyl)oxy]methyl}-1,3,4-thiadiazol-2-yl)amino]carbonyl}-3,4-dihydro-2(1H)-isoquinolinecarboxylate, (Intermediate 120) (14 g, 28 mmol) was added. The reaction mixture was stirred at room temperature for 2 hours. The resulting precipitate was filtered, washed with EtOAc and dried to give the title compound as a white solid after triturating with acetonitrile (11.8 g, 97%).

HRMS calculated for C₁₉H₁₇ClN₄O₂S (M+H)⁺ 401.0839, found: 401.0850, Rt: 2.34 min. MP: 300.4° C.

The following Examples were similarly prepared by a method analogous to that described for Example 1

TABLE 20

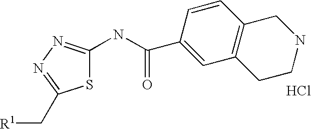

| | | From | |
|---|---|---|---|
| Example No. | R¹ | Intermediate No. | Physical data |
| 2 N-[5-(1- naphthalenylmethyl)- 1,3,4-thiadiazol-2-yl]- 1,2,3,4-tetrahydro-6- isoquinolinecarboxamide hydrochloride | 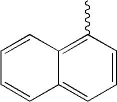 | 121 1,1-dimethylethyl 6-({[5- (1-naphthalenylmethyl)- 1,3,4-thiadiazol-2- yl]amino}carbonyl)-3,4- dihydro-2(1H)- isoquinolinecarboxylate | HRMS (M + H)⁺: calculated for C₂₃H₂₁N₄OS Theo: 401.1436, Found: 401.1463, Rt: 2.53 min. MP: 335.6° C. |
| 3 N-(5-{[(3,4- dichlorophenyl)oxy]methyl}- 1,3,4-thiadiazol-2-yl)- 1,2,3,4-tetrahydro-6- isoquinolinecarboxamide hydrochloride | 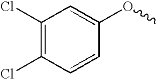 | 137 1,1-dimethylethyl 6-{[(5- {[(3,4- dichlorophenyl)oxy]meth- yl}-1,3,4-thiadiazol-2- yl)amino]carbonyl}-3,4- dihydro-2(1H)- isoquinolinecarboxylate | HRMS (M + H)⁺: calculated for C₁₉H₁₆Cl₂N₄O₂S Theo: 435.0449 Found: 435.0486 Rt: 2.22 min MP: 276° C. |
| 4 N-(5-{[(4- chlorophenyl)oxy]methyl}- 1,3,4-thiadiazol-2-yl)- 1,2,3,4-tetrahydro-6- isoquinolinecarboxamide hydrochloride | 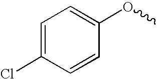 | 139 1,1-dimethylethyl 6-{[(5- {[(4- chlorophenyl)oxy]methyl}- 1,3,4-thiadiazol-2- yl)amino]carbonyl}-3,4- dihydro-2(1H)- isoquinolinecarboxylate | HRMS (M + H)⁺: calculated for C₁₉H₁₇ClN₄O₂S Theo: 401.0839 Found: 401.0819 Rt: 2.06 min MP: 277° C. |
| 5 N-[5-(phenylmethyl)-1,3,4- thiadiazol-2-yl]-1,2,3,4- tetrahydro-6- isoquinolinecarboxamide hydrochloride | 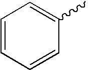 | 133 1,1-dimethylethyl 6-({[5- (phenylmethyl)-1,3,4- thiadiazol-2- yl]amino}carbonyl)-3,4- dihydro-2(1H)- isoquinolinecarboxylate | HRMS (M + H)⁺: calculated for C₁₉H₁₈N₄OS. Theo: 351.1280 Found: 351.1311 Rt: 1.90 min MP: 291.0° C. |
| 6 N-{5-[(4- chlorophenyl)methyl]- 1,3,4-thiadiazol-2-yl}- 1,2,3,4-tetrahydro-6- isoquinolinecarboxamide hydrochloride | 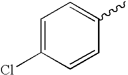 | 134 1,1-dimethylethyl 6-[({5- [(4-chlorophenyl)methyl]- 1,3,4-thiadiazol-2- yl}amino)carbonyl]-3,4- dihydro-2(1H)- isoquinolinecarboxylate | HRMS (M + H)⁺: calculated for C₁₉H₁₇ClN₄OS Theo: 385.0890 Found: 385.0896 Rt: 2.07 min MP: 285.6° C. |
| 7 -{5-[(3,4- dichlorophenyl)methyl]- 1,3,4-thiadiazol-2-yl}- 1,2,3,4-tetrahydro-6- isoquinolinecarboxamide hydrochloride | 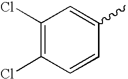 | 135 1,1-dimethylethyl 6-[({5- [(3,4- dichlorophenyl)methyl]- 1,3,4-thiadiazol-2- yl}amino)carbonyl]-3,4- dihydro-2(1H)- isoquinolinecarboxylate | HRMS (M + H)⁺: calculated for C₁₉H₁₆Cl₂N₄OS Theo: 419.0500 Found: 419.0507 Rt: 2.19 min MP: 287° C. |
| 8 N-[5-(2-thienylmethyl)- 1,3,4-thiadiazol-2-yl]- 1,2,3,4-tetrahydro-6- isoquinolinecarboxamide hydrochloride | 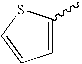 | 122 1,1-dimethylethyl 6-({[5- (2-thienylmethyl)-1,3,4- thiadiazol-2- yl]amino}carbonyl)-3,4- dihydro-2(1H)- isoquinolinecarboxylate | HRMS (M + H)⁺: calculated for C₁₇H₁₆N₄OS₂ Theo: 357.0844 Found: 357.0879 Rt: 2.1 min MP: 334° C. |
| 9 N-[5-(2- naphthalenylmethyl)- 1,3,4-thiadiazol-2-yl]- 1,2,3,4-tetrahydro-6- isoquinolinecarboxamide hydrochloride | 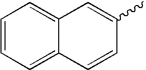 | 123 1,1-dimethylethyl 6-({[5- (2-naphthalenylmethyl)- 1,3,4-thiadiazol-2- yl]amino}carbonyl)-3,4- dihydro-2(1H)- isoquinolinecarboxylate | HRMS (M + H)⁺: calculated for C₂₃H₂₀N₄OS Theo: 401.1436 Found: 401.1438 Rt: 2.37 min MP: 308.7° C. |
| 10 N-[5-(cyclohexylmethyl)- 1,3,4-thiadiazol-2-yl]- 1,2,3,4-tetrahydro-6- isoquinolinecarboxamide hydrochloride | 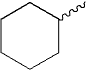 | 124 1,1-dimethylethyl 6-({[5- (cyclohexylmethyl)-1,3,4- thiadiazol-2- yl]amino}carbonyl)-3,4- dihydro-2(1H)- isoquinolinecarboxylate | HRMS (M + H)⁺: calculated for C₁₉H₂₄N₄OS Theo: 357.1749 Found: 357.1736 Rt: 2.37 min MP: 317.8° C. |
| 11 N-[5-(2-phenylethyl)-1,3,4- thiadiazol-2-yl]-1,2,3,4- tetrahydro-6- isoquinolinecarboxamide hydrochloride | 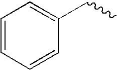 | 125 1,1-dimethylethyl 6-({[5- (2-phenylethyl)-1,3,4- thiadiazol-2- yl]amino}carbonyl)-3,4- dihydro-2(1H)- isoquinolinecarboxylate | HRMS (M + H)⁺: calculated for C₂₀H₂₀N₄OS Theo: 365.1436 Found: 365.1436 Rt: 2.20 min MP: 312.1° C. |
| 12 N-[5-(1H-indol-3- ylmethyl)-1,3,4-thiadiazol- 2-yl]-1,2,3,4-tetrahydro-6- isoquinolinecarboxamide hydrochloride | 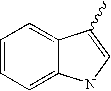 | 126 1,1-dimethylethyl 6-({[5- (1H-indol-3-ylmethyl)- 1,3,4-thiadiazol-2- yl]amino}carbonyl)-3,4- dihydro-2(1H)- isoquinolinecarboxylate | HRMS (M + H)⁺: calculated for C₂₁H₁₉N₅OS Theo: 389.1310 Found: 389.1394 Rt: 2.12 min MP: 313.1° C. |
| 13 N-(5-{[(2,5- dichlorophenyl)oxy]methyl}- 1,3,4-thiadiazol-2-yl)- 1,2,3,4-tetrahydro-6- isoquinolinecarboxamide hydrochloride | 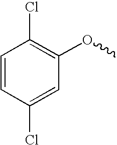 | 127 1,1-dimethylethyl 6-{[(5- {[(2,5- dichlorophenyl)oxy]meth- yl}-1,3,4-thiadiazol-2- yl)amino]carbonyl}-3,4- dihydro-2(1H)- isoquinolinecarboxylate | HRMS (M + H)⁺: calculated for C₁₉H₁₆Cl₂N₄O₂S Theo: 435.0449 Found: 435.0474 Rt: 2.62 min MP: 330.1° C.: |
| 14 N-{5-[(1- naphthalenyloxy)methyl]- 1,3,4-thiadiazol-2-yl}- 1,2,3,4-tetrahydro-6- isoquinolinecarboxamide hydrochloride | 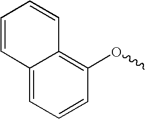 | 147 1,1-dimethylethyl 6-[({5- [(1- naphthalenyloxy)methyl]- 1,3,4-thiadiazol-2- yl}amino)carbonyl]-3,4- dihydro-2(1H)- isoquinolinecarboxylate | HRMS (M + H)⁺: calculated for C₂₃H₂₀N₄O₂S Theo: 417.1385 Found: 417.1882 Rt: 2.62 min MP: 295.1° C. |
| 15 N-(5-{[(2-chloro-4- fluorophenyl)oxy]methyl}- 1,3,4-thiadiazol-2-yl)- 1,2,3,4-tetrahydro-6- isoquinolinecarboxamide hydrochloride | 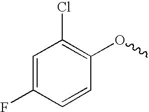 | 140 1,1-dimethylethyl 6-{[(5- {[(2-chloro-4- fluorophenyl)oxy]methyl}- 1,3,4-thiadiazol-2- yl)amino]carbonyl}-3,4- dihydro-2(1H)- isoquinolinecarboxylate | HRMS (M + H)⁺: calculated for C₁₉H₁₆ClFN₄O₂S Theo: 419.0745 Found: 419.0780 Rt: 2.43 min MP: 298° C.: |
| 16 N-(5-{[(2-chloro-5- fluorophenyl)oxy]methyl}- 1,3,4-thiadiazol-2-yl)- 1,2,3,4-tetrahydro-6- isoquinolinecarboxamide hydrochloride | 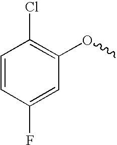 | 141 1,1-dimethylethyl 6-{[(5- {[(2-chloro-5- fluorophenyl)oxy]methyl}- 1,3,4-thiadiazol-2- yl)amino]carbonyl}-3,4- dihydro-2(1H)- isoquinolinecarboxylate | HRMS (M + H)⁺: calculated for C₁₉H₁₆ClFN₄O₂S Theo: 419.0745 Found: 419.0746 Rt: 2.47 min MP: 346.2° C.: |
| 17 N-[5-(1-benzothien-3- ylmethyl)-1,3,4-thiadiazol- 2-yl]-1,2,3,4-tetrahydro-6- isoquinolinecarboxamide hydrochloride | 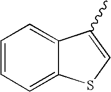 | 142 1,1-dimethylethyl 6-({[5- (1-benzothien-3- ylmethyl)-1,3,4- thiadiazol-2- yl]amino}carbonyl)-3,4- dihydro-2(1H)- isoquinolinecarboxylate | HRMS (M + H)⁺: calculated for C₂₁H₁₈FN₄O₂S Theo: 407.1000 Found: 407.1023 Rt: 2.47 min MP: 325.1° C.: |
| 18 N-[5-(3-thienylmethyl)- 1,3,4-thiadiazol-2-yl]- 1,2,3,4-tetrahydro-6- isoquinolinecarboxamide hydrochloride | 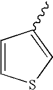 | 143 1,1-dimethylethyl 6-({[5- (3-thienylmethyl)-1,3,4- thiadiazol-2- yl]amino}carbonyl)-3,4- dihydro-2(1H)- isoquinolinecarboxylate | HRMS (M + H)⁺: calculated for C₁₇H₁₆N₄OS₂ Theo: 357.0844 Found: 357.0863 Rt: 2.12 min MP: 331.9° C.: |
| 19 N-{5-[2-(1- naphthalenyl)ethyl]-1,3,4- thiadiazol-2-yl}-1,2,3,4- tetrahydro-6- isoquinolinecarboxamide hydrochloride | 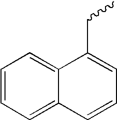 | 144 1,1-dimethylethyl 6-[({5- [2-(1-naphthalenyl)ethyl]- 1,3,4-thiadiazol-2- yl}amino)carbonyl]-3,4- dihydro-2(1H)- isoquinolinecarboxylate | HRMS (M + H)⁺: calculated for C₂₄H₂₂N₄OS Theo: 415.1592 Found: 415.1593 Rt: 2.66 min MP: 333.2° C. |
| 20 N-{5-[2-(2- chlorophenyl)ethyl]-1,3,4- thiadiazol-2-yl}-1,2,3,4- tetrahydro-6- isoquinolinecarboxamide hydrochloride | 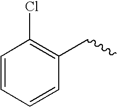 | 145 1,1-dimethylethyl 6-[({5- [2-(2-chlorophenyl)ethyl]- 1,3,4-thiadiazol-2- yl}amino)carbonyl]-3,4- dihydro-2(1H)- isoquinolinecarboxylate | HRMS (M + H)⁺: calculated for C₂₀H₁₉ClN₄OS Theo: 399.1046 Found: 399.1072 Rt: 2.53 min MP: 318.2° C. |
| 21 N-{5-[(2- bromophenyl)methyl]- 1,3,4-thiadiazol-2-yl}- 1,2,3,4-tetrahydro-6- isoquinolinecarboxamide hydrochloride | 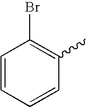 | 128 1,1-dimethylethyl 6-[({5- [(2-bromophenyl)methyl]- 1,3,4-thiadiazol-2- yl}amino)carbonyl]-3,4- dihydro-2(1H)- isoquinolinecarboxylate | HRMS (M + H)⁺: calculated for C₁₉H₁₇BrN₄OS Theo: 429.0385 Found: 429.0413 Rt: 2.40 min MP: 309.7° C. |
| 22 N-(5-{[(2- fluorophenyl)oxy]methyl}- 1,3,4-thiadiazol-2-yl)- 1,2,3,4-tetrahydro-6- isoquinolinecarboxamide hydrochloride | 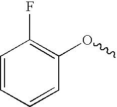 | 130 1,1-dimethylethyl 6-{[(5- {[(2- fluorophenyl)oxy]methyl}- 1,3,4-thiadiazol-2- yl)amino]carbonyl}-3,4- dihydro-2(1H)- isoquinolinecarboxylate | HRMS (M + H)⁺: calculated for C₁₉H₁₇FN₄O₂S Theo: 385.1134 Found: 385.1155 Rt: 2.27 min MP: 250-260° C. |
| 23 N-(5-{[(3- fluorophenyl)oxy]methyl}- 1,3,4-thiadiazol-2-yl)- 1,2,3,4-tetrahydro-6- isoquinolinecarboxamide hydrochloride | 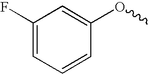 | 131 1,1-dimethylethyl 6-{[(5- {[(3- fluorophenyl)oxy]methyl}- 1,3,4-thiadiazol-2- yl)amino]carbonyl}-3,4- dihydro-2(1H)- isoquinolinecarboxylate | HRMS (M + H)⁺: calculated for C₁₉H₁₇FN₄O₂S Theo: 385.1134 Found: 385.1168 Rt: 2.33 min MP: 250-260° C. |
| 24 N-(5-{[(4- fluorophenyl)oxy]methyl}- 1,3,4-thiadiazol-2-yl)- 1,2,3,4-tetrahydro-6- isoquinolinecarboxamide hydrochloride | 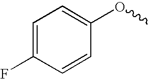 | 132 1,1-dimethylethyl 6-{[(5- {[(4- fluorophenyl)oxy]methyl}- 1,3,4-thiadiazol-2- yl)amino]carbonyl}-3,4- dihydro-2(1H)- isoquinolinecarboxylate | HRMS (M + H)⁺: calculated for C₁₉H₁₇FN₄O₂SI Theo: 385.1134 Found: 385.1168 Rt: 2.25 min MP: 240-250° C. |
| 25 N-(5-{[(3- chlorophenyl)oxy]methyl}- 1,3,4-thiadiazol-2-yl)- 1,2,3,4-tetrahydro-6- isoquinolinecarboxamide hydrochloride | 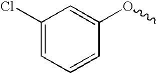 | 136 1,1-dimethylethyl 6-{[(5- {[(3- chlorophenyl)oxy]methyl}- 1,3,4-thiadiazol-2- yl)amino]carbonyl}-3,4- dihydro-2(1H)- isoquinolinecarboxylate | HRMS (M + H)⁺: calculated for C₁₉H₁₇ClN₄O₂S. Theo: 401.0839 Found: 401.0847 Rt: 2.55 min MP: 281.7° C. |
| 26 N-[5-({[2- (trifluoromethyl)phenyl]oxy} methyl)-1,3,4-thiadiazol- 2-yl]-1,2,3,4-tetrahydro-6- isoquinolinecarboxamide hydrochloride | 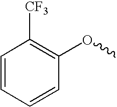 | 129 1,1-dimethylethyl 6-({[5- ({[2- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)⁺: calculated for C₂₀H₁₇F₃N₄O₂S Theo: 435.1102 Found: 435.1144 Rt: 2.50 min MP: 300.2° C. |
| 27 N-[5-({[3- (trifluoromethyl)phenyl]oxy} methyl)-1,3,4-thiadiazol- 2-yl]-1,2,3,4-tetrahydro-6- isoquinolinecarboxamide hydrochloride | 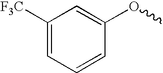 | 138 1,1-dimethylethyl 6-({[5- ({[3- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)⁺: calculated for C₂₀H₁₇F₃N₄O₂S Theo: 435.1102 Found: 435.1101 Rt: 2.50 min MP: 298.9° C. |
| 28 N-(5-{[3- (trifluoromethyl)phenyl]meth- yl}-1,3,4-thiadiazol-2-yl)- 1,2,3,4-tetrahydro-6- isoquinolinecarboxamide hydrochloride | 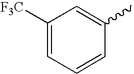 | 148 1,1-dimethylethyl 6-{[(5- {[3- (trifluoromethyl)phenyl] methyl}-1,3,4-thiadiazol-2- yl)amino]carbonyl}-3,4- dihydro-2(1H)- isoquinolinecarboxylate | HRMS (M + H)⁺: calculated for C₂₀H₁₇F₃N₄OS Theo: 419.1153 Found: 419.1161 Rt: 2.41 min MP: 290.7° C. |
| 29 N-{5-[(5,6,7,8-tetrahydro- 1- naphthalenyloxy)methyl]- 1,3,4-thiadiazol-2-yl}- 1,2,3,4-tetrahydro-6- isoquinolinecarboxamide hydrochloride | 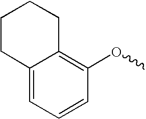 | 146 1,1-dimethylethyl 6-[({5- [(5,6,7,8-tetrahydro-1- naphthalenyloxy)methyl]- 1,3,4-thiadiazol-2- yl}amino)carbonyl]-3,4- dihydro-2(1H)- isoquinolinecarboxylate | HRMS (M + H)⁺: calculated for C₂₃H₂₄N₄O₂S Theo: 421.1698 Found: 421.1689 Rt: 2.81 min MP: 297.6° C. |
| 30 N-{5-[(2- chlorophenyl)methyl]- 1,3,4-thiadiazol-2-yl}- 1,2,3,4-tetrahydro-6- isoquinolinecarboxamide hydrochloride | 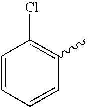 | 149 1,1-dimethylethyl 6-[({5- [(2-chlorophenyl)methyl]- 1,3,4-thiadiazol-2- yl}amino)carbonyl]-3,4- dihydro-2(1H)- isoquinolinecarboxylate | HRMS (M + H)⁺: calculated for C₁₉H₁₇ClN₄OS Theo: 385.0811 Found: 385.0920 Rt: 2.28 min MP: 287° C. |
| 31 N-(5-{[2- (trifluoromethyl)phenyl]meth- yl}-1,3,4-thiadiazol-2-yl)- 1,2,3,4-tetrahydro-6- isoquinolinecarboxamide hydrochloride | 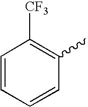 | 150 1,1-dimethylethyl 6-{[(5- {[2- (trifluoromethyl)phenyl] methyl}-1,3,4-thiadiazol-2- yl)amino]carbonyl}-3,4- dihydro-2(1H)- isoquinolinecarboxylate | HRMS (M + H)⁺: calculated for C₂₀H₁₇F₃N₄OS Theo: 419.1153 Found: 419.1185 Rt: 2.37 min MP: 292.7° C. |
| 32 N-[5-({[4- (methyloxy)phenyl]oxy}meth- yl)-1,3,4-thiadiazol-2-yl]- 1,2,3,4-tetrahydro-6- isoquinolinecarboxamide hydrochloride | 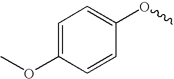 | 151 1,1-dimethylethyl 6-({[5- ({[4- (methyloxy)phenyl]oxy} methyl)-1,3,4-thiadiazol-2- yl]amino}carbonyl)-3,4- dihydro-2(1H)- isoquinolinecarboxylate | HRMS (M + H)⁺: calculated for C₂₀H₂₀N₄O₃S Theo: 397.1334 Found: 397.1318 Rt: 2.16 min MP: 271° C. |
| 33 N-{5-[(2- biphenylyloxy)methyl]- 1,3,4-thiadiazol-2-yl}- 1,2,3,4-tetrahydro-6- isoquinolinecarboxamide hydrochloride | 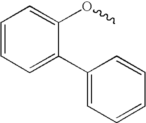 | 152 1,1-dimethylethyl 6-[({5- [(2- biphenylyloxy)methyl]- 1,3,4-thiadiazol-2- yl}amino)carbonyl]-3,4- dihydro-2(1H)- isoquinolinecarboxylate | HRMS (M + H)⁺: calculated for C₂₅H₂₂N₄O₂S Theo: 443.1542 Found: 443.1524 Rt: 2.58 min MP: 278.2° C. |
| 34 N-(5-{[4- (trifluoromethyl)phenyl]meth- yl}-1,3,4-thiadiazol-2-yl)- 1,2,3,4-tetrahydro-6- isoquinolinecarboxamide hydrochloride | 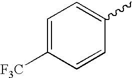 | 153 1,1-dimethylethyl 6-{[(5- {[4- (trifluoromethyl)phenyl] methyl}-1,3,4-thiadiazol-2- yl)amino]carbonyl}-3,4- dihydro-2(1H)- isoquinolinecarboxylate | HRMS (M + H)⁺: calculated for C₂₀H₁₇F₃N₄OS Theo: 419.1153 Found: 419.1183 Rt: 2.41 min MP: 284° C. |
| 35 N-{5-[({5-chloro-2-[(2- methylpropyl)oxy]phenyl} oxy)methyl]-1,3,4- thiadiazol-2-yl}-1,2,3,4- tetrahydro-6- isoquinolinecarboxamide hydrochloride | 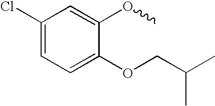 | 154 1,1-dimethylethyl 6-[({5- [({5-chloro-2-[(2- methylpropyl)oxy]phenyl} oxy)methyl]-1,3,4- thiadiazol-2- yl}amino)carbonyl]-3,4- dihydro-2(1H)- isoquinolinecarboxylate | HRMS (M + H)⁺: calculated for C₂₃H₂₅ClN₄O₃S Theo: 473.1414 Found: 473.1448 Rt: 2.76 min MP: 276.1° C. |
| 36 N-{5-[(4- fluorophenyl)methyl]- 1,3,4-thiadiazol-2-yl}- 1,2,3,4-tetrahydro-6- isoquinolinecarboxamide hydrochloride | 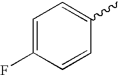 | 155 1,1-dimethylethyl 6-[({5- [(4-fluorophenyl)methyl]- 1,3,4-thiadiazol-2- yl}amino)carbonyl]-3,4- dihydro-2(1H)- isoquinolinecarboxylate | HRMS (M + H)⁺: calculated for C₁₉H₁₇FN₄OS Theo: 369.1185 Found: 369.1220 Rt: 2.19 min MP: 294.6° C. |
| 37 N-[5-({[2- (methyloxy)phenyl]oxy}meth- yl)-1,3,4-thiadiazol-2-yl]- 1,2,3,4-tetrahydro-6- isoquinolinecarboxamide hydrochloride | 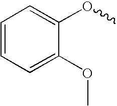 | 156 1,1-dimethylethyl 6-({[5- ({[2- (methyloxy)phenyl]oxy} methyl)-1,3,4-thiadiazol-2- yl]amino}carbonyl)-3,4- dihydro-2(1H)- isoquinolinecarboxylate | HRMS (M + H)⁺: calculated for C₂₀H₂₀N₄O₃S Theo: 397.1334 Found: 397.1361 Rt: 2.07 min MP: 279.7° C. |
| 38 N-{5-[(1-methyl-1H-indol- 3-yl)methyl]-1,3,4- thiadiazol-2-yl}-1,2,3,4- tetrahydro-6- isoquinolinecarboxamide hydrochloride | 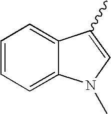 | 157 1,1-dimethylethyl 6-[({5- [(1-methyl-1H-indol-3- yl)methy]-1,3,4- thiadiazol-2- yl}amino)carbonyl]-3,4- dihydro-2(1H)- isoquinolinecarboxylate | HRMS (M + H)⁺: calculated for C₂₂H₂₁N₅OS Theo: 404.1545 Found: 404.1576 Rt: 2.31 min MP: 311.5° C. |
| 39 N-[5-(3-pyridinylmethyl)- 1,3,4-thiadiazol-2-yl]- 1,2,3,4-tetrahydro-6- isoquinolinecarboxamide hydrochloride | 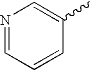 | 158 1,1-dimethylethyl 6-({[5- (3-pyridinylmethyl)-1,3,4- thiadiazol-2- yl]amino}carbonyl)-3,4- dihydro-2(1H)- isoquinolinecarboxylate | HRMS (M + H)⁺: calculated for C₁₈H₁₇N₅OS Theo: 352.1232 Found: 352.1206 Rt: 1.70 min |
| 40 N-[5-(5,6,7,8-tetrahydro-2- naphthalenylmethyl)- 1,3,4-thiadiazol-2-yl]- 1,2,3,4-tetrahydro-6- isoquinolinecarboxamide hydrochloride | 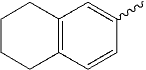 | 159 1,1-dimethylethyl 6-({[5- (5,6,7,8-tetrahydro-2- naphthalenylmethyl)- 1,3,4-thiadiazol-2- yl]amino}carbonyl)-3,4- dihydro-2(1H)- isoquinolinecarboxylate | HRMS (M + H)⁺: calculated for C₂₃H₂₄N₄OS Theo: 405.1744 Found: 405.1725 Rt: 2.60 min MP: 313.6° C. |
| 41 N-[5-(3,4-dihydro-2H- chromen-6-ylmethyl)- 1,3,4-thiadiazol-2-yl]- 1,2,3,4-tetrahydro-6- isoquinolinecarboxamide hydrochloride | 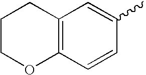 | 160 1,1-dimethylethyl 6-({[5- (3,4-dihydro-2H- chromen-6-ylmethyl)- 1,3,4-thiadiazol-2- yl]amino}carbonyl)-3,4- dihydro-2(1H)- isoquinolinecarboxylate | HRMS (M + H)⁺: calculated for C₂₂H₂₂N₄O₂S Theo: 407.1542 Found: 407.1560 Rt: 2.26 min MP: 323° C. |
| 42 N-(5-{2-[(2- chlorophenyl)oxy]ethyl}- 1,3,4-thiadiazol-2-yl)- 1,2,3,4-tetrahydro-6- isoquinolinecarboxamide hydrochloride | 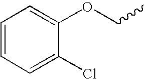 | 161 1,1-dimethylethyl 6-{[(5- {2-[(2- chlorophenyl)oxy]ethyl}- 1,3,4-thiadiazol-2- yl)amino]carbonyl}-3,4- dihydro-2(1H)- isoquinolinecarboxylate | HRMS (M + H)⁺: calculated for C₂₀H₁₉ClN₄O₂S Theo: 415.0995 Found: 415.1005 Rt: 2.32 min |
| 43 N-(5-{[(2,4- dichlorophenyl)oxy]methyl}- 1,3,4-thiadiazol-2-yl)- 1,2,3,4-tetrahydro-6- isoquinolinecarboxamide hydrochloride | 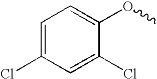 | 162 1,1-dimethylethyl 6-{[(5- {[(2,4- dichlorophenyl)oxy]meth- yl}-1,3,4-thiadiazol-2- yl)amino]carbonyl}-3,4- dihydro-2(1H)- isoquinolinecarboxylate | HRMS (M + H)⁺: calculated for C₁₉H₁₆Cl₂N₄O₂S Theo: 435.0449 Found: 435.0492 Rt: 2.44 min MP: 295.1° C. |
| 44 N-{5-[(2′-chloro-2- biphenylyl)methyl]-1,3,4- thiadiazol-2-yl}-1,2,3,4- tetrahydro-6- isoquinolinecarboxamide hydrochloride | 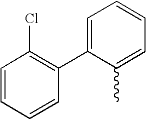 | 163 1,1-dimethylethyl 6-[({5- [(2′-chloro-2- biphenylyl)methyl]-1,3,4- thiadiazol-2- yl}amino)carbonyl]-3,4- dihydro-2(1H)- isoquinolinecarboxylate | HRMS (M + H)⁺: calculated for C₂₅H₂₁ClN₄OS Theo: 461.1203 Found: 461.1229 Rt: 2.78 min MP: 320.4° C. |
| 45 N-{5-[(2- fluorophenyl)methyl]- 1,3,4-thiadiazol-2-yl}- 1,2,3,4-tetrahydro-6- isoquinolinecarboxamide hydrochloride | 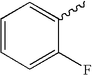 | 164 1,1-dimethylethyl 6-[({5- [(2-fluorophenyl)methyl]- 1,3,4-thiadiazol-2- yl}amino)carbonyl]-3,4- dihydro-2(1H)- isoquinolinecarboxylate | HRMS (M + H)⁺: calculated for C₁₉H₁₇FN₄OS Theo: 369.1185 Found: 369.1164 Rt: 2.15 min MP: 319° C. |
| 46 N-{5-[(3- chlorophenyl)methyl]- 1,3,4-thiadiazol-2-yl}- 1,2,3,4-tetrahydro-6- isoquinolinecarboxamide hydrochloride | 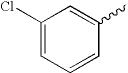 | 213 1,1-dimethylethyl 6-[({5- [(3-chlorophenyl)methyl]- 1,3,4-thiadiazol-2- yl}amino)carbonyl]-3,4- dihydro-2(1H)- isoquinolinecarboxylate | HRMS (M + H)+: calculated for C₁₉H₁₇ClN₄OS Theo: 385.0890 Found: 385.0910 Rt: 2.34 min MP: 302.2° C. |
| 47 N-(5-{[(2,6- dichlorophenyl)oxy]methyl}- 1,3,4-thiadiazol-2-yl)- 1,2,3,4-tetrahydro-6- isoquinolinecarboxamide hydrochloride | 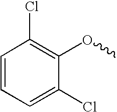 | 214 1,1-dimethylethyl 6-{[(5- {[(2,6- dichlorophenyl)oxy]meth- yl}-1,3,4-thiadiazol-2- yl)amino]carbonyl}-3,4- dihydro-2(1H)- isoquinolinecarboxylate | HRMS (M + H)+: calculated for C₁₉H₁₆Cl₂N₄O₂S Theo: 435.0449 Found: 435.0453 Rt: 2.40 min MP: 304.8° C. |
| 48 N-(5-{[(2- methylphenyl)oxy]methyl}- 1,3,4-thiadiazol-2-yl)- 1,2,3,4-tetrahydro-6- isoquinolinecarboxamide hydrochloride | 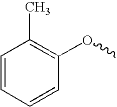 | 215 1,1-dimethylethyl 6-{[(5- {[(2- methylphenyl)oxy]methyl}- 1,3,4-thiadiazol-2- yl)amino]carbonyl}-3,4- dihydro-2(1H)- isoquinolinecarboxylate | HRMS (M + H)+: calculated for C₂₀H₂₀N₄O₂S Theo: 381.1385 Found: 381.1382 Rt: 2.29 min MP: 311.3° C. |
| 49 N-(5-{[(3,4- dimethylphenyl)oxy]methyl}- 1,3,4-thiadiazol-2-yl)- 1,2,3,4-tetrahydro-6- isoquinolinecarboxamide hydrochloride | 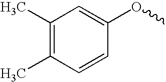 | 216 1,1-dimethylethyl 6-{[(5- {[(3,4- dimethylphenyl)oxy]meth- yl}-1,3,4-thiadiazol-2- yl)amino]carbonyl}-3,4- dihydro-2(1H)- isoquinolinecarboxylate | HRMS (M + H)+: calculated for C₂₁H₂₂N₄O₂S Theo: 395.1542 Found: 395.1538 Rt: 2.37 min MP: 291° C. |
| 50 N-{5-[(2,4- dichlorophenyl)methyl]- 1,3,4-thiadiazol-2-yl}- 1,2,3,4-tetrahydro-6- isoquinolinecarboxamide hydrochloride | 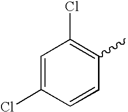 | 217 1,1-dimethylethyl 6-[({5- [(2,4- dichlorophenyl)methyl]- 1,3,4-thiadiazol-2- yl}amino)carbonyl]-3,4- dihydro-2(1H)- isoquinolinecarboxylate | HRMS (M + H)+: calculated for C₁₉H₁₆Cl₂N₄OS Theo: 419.0550 Found: 419.0529 Rt: 2.51 min MP: 308.7° C. |
| 51 N-[5-({2- [(trifluoromethyl)oxy]phen- yl}methyl)-1,3,4- thiadiazol-2-yl]-1,2,3,4- tetrahydro-6- isoquinolinecarboxamide | 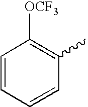 | 218 1,1-dimethylethyl 6-({[5- ({2- [(trifluoromethyl)oxy]phen- yl}methyl)-1,3,4- thiadiazol-2- yl]amino}carbonyl)-3,4- dihydro-2(1H)- isoquinolinecarboxylate | HRMS (M + H)+: calculated for C₂₀H₁₇F₃N₄O₂S Theo: 435.1103 Found: 435.1113 Rt: 2.46 min MP: 293.9° C. |
| 52 N-(5-{[(2-chloro-3,5- difluorophenyl)oxy]methyl}- 1,3,4-thiadiazol-2-yl)- 1,2,3,4-tetrahydro-6- isoquinolinecarboxamide hydrochloride | 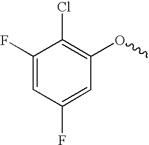 | 219 1,1-dimethylethyl 6-{[(5- {[(2-chloro-3,5- difluorophenyl)oxy]methyl}- 1,3,4-thiadiazol-2- yl)amino]carbonyl}-3,4- dihydro-2(1H)- isoquinolinecarboxylate | HRMS (M + H)+: calculated for C₁₉H₁₅ClF₂N₄O₂S Theo: 437.0650 Found: 437.0656 Rt: 2.00 min MP > 260° C. |
| 53 N-(5-{[(2-chloro-6- fluorophenyl)oxy]methyl}- 1,3,4-thiadiazol-2-yl)- 1,2,3,4-tetrahydro-6- isoquinolinecarboxamide hydrochloride | 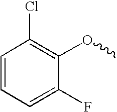 | 220 1,1-dimethylethyl 6-{[(5- {[(2-chloro-6- fluorophenyl)oxy]methyl}- 1,3,4-thiadiazol-2- yl)amino]carbonyl}-3,4- dihydro-2(1H)- isoquinolinecarboxylate | HRMS (M + H)+: calculated for C₁₉H₁₆ClFN₄O₂S Theo: 419.0745 Found: 419.0750 Rt: 2.31 min MP: 318.9° C. |
| 54 N-[5-({[2-chloro-3- (trifluoromethyl)phenyl]oxy} methyl)-1,3,4-thiadiazol- 2-yl]-1,2,3,4-tetrahydro-6- isoquinolinecarboxamide hydrochloride | 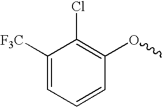 | 221 1,1-dimethylethyl 6-({[5- ({[2-chloro-3- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)+: calculated for C₂₀H₁₆ClF₃N₄O₂S Theo: 469.0713 Found: 469.0748 Rt: 2.16 min MP: 312.9° C. |
| 55 N-(5-{[(2,4,5- trichlorophenyl)oxy]methyl}- 1,3,4-thiadiazol-2-yl)- 1,2,3,4-tetrahydro-6- isoquinolinecarboxamide hydrochloride | 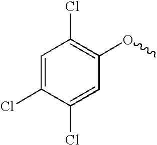 | 222 1,1-dimethylethyl 6-{[(5- {[(2,4,5- trichlorophenyl)oxy]meth- yl}-1,3,4-thiadiazol-2- yl)amino]carbonyl}-3,4- dihydro-2(1H)- isoquinolinecarboxylate | HRMS (M + H)+: calculated for C₁₉H₁₅Cl₃N₄O₂S Theo: 469.0060 Found: 469.0037 Rt: 2.66 min MP: 291.8° C. |
| 56 N-[5-({[2-chloro-5- (trifluoromethyl)phenyl]oxy} methyl)-1,3,4-thiadiazol- 2-yl]-1,2,3,4-tetrahydro-6- isoquinolinecarboxamide hydrochloride | 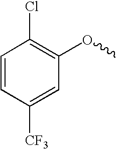 | 223 1,1-dimethylethyl 6-({[5- ({[2-chloro-5- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)+: calculated for C₂₀H₁₆ClF₃N₄O₂S Theo: 469.0713 Found: 469.0674 Rt: 2.57 min MP: 317.5° C. |
| 57 N-{5-[(4-chloro-2- fluorophenyl)methyl]- 1,3,4-thiadiazol-2-yl}- 1,2,3,4-tetrahydro-6- isoquinolinecarboxamide hydrochloride | 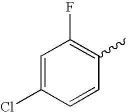 | 224 1,1-dimethylethyl 6-({[5- ({[2-chloro-5- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)+: calculated for C₁₉H₁₆ClFN₄OS Theo: 403.0796 Found: 403.0764 Rt: 2.31 min MP: 300.2° C. |
| 58 N-(5-{[4-fluoro-2- (trifluoromethyl)phenyl]meth- yl}-1,3,4-thiadiazol-2-yl)- 1,2,3,4-tetrahydro-6- isoquinolinecarboxamide hydrochloride | 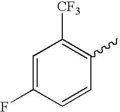 | 225 1,1-dimethylethyl 6-{[(5- {[4-fluoro-2- (trifluoromethyl)phenyl] methyl}-1,3,4-thiadiazol-2- yl)amino]carbonyl}-3,4- dihydro-2(1H)- isoquinolinecarboxylate | HRMS (M + H)+: calculated for C₂₀H₁₆F₄N₄OS Theo: 437.1059 Found: 437.1079 Rt: 2.47 min MP: 306.2° C. |
| 59 N-(5-{[5-chloro-2- (trifluoromethyl)phenyl]meth- yl}-1,3,4-thiadiazol-2-yl)- 1,2,3,4-tetrahydro-6- isoquinolinecarboxamide hydrochloride | 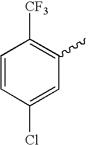 | 226 1,1-dimethylethyl 6-{[(5- {[5-chloro-2- (trifluoromethyl)phenyl] methyl}-1,3,4-thiadiazol-2- yl)amino]carbonyl}-3,4- dihydro-2(1H)- isoquinolinecarboxylate | HRMS (M + H)+: calculated for C₂₀H₁₆ClF₃N₄OS Theo: 453.0764 Found: 453.0772 Rt: 2.57 min MP: 304.7° C. |
| 60 N-[5-({[4-fluoro-2- (trifluoromethyl)phenyl]oxy} methyl)-1,3,4-thiadiazol- 2-yl]-1,2,3,4-tetrahydro-6- isoquinolinecarboxamide hydrochloride | 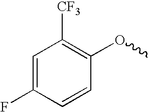 | 227 1,1-dimethylethyl 6-({[5- ({[4-fluoro-2- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)+: calculated for C₂₀H₁₆F₄N₄O₂S Theo: 453.1008 Found: 453.1029 Rt: 2.43 min MP: 299.4° C. |
| 61 N-[5-({[2-chloro-4- (trifluoromethyl)phenyl]oxy} methyl)-1,3,4-thiadiazol- 2-yl]-1,2,3,4-tetrahydro-6- isoquinolinecarboxamide hydrochloride | 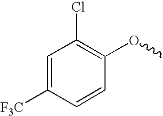 | 228 1,1-dimethylethyl 6-({[5- ({[2-chloro-4- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)+: calculated for C₂₀H₁₆ClF₃N₄O₂S Theo: 469.0713 Found: 469.0720 Rt: 2.58 min MP: 326.3° C. |
| 62 N-(5-{[(3-chloro-5- fluorophenyl)oxy]methyl}- 1,3,4-thiadiazol-2-yl)- 1,2,3,4-tetrahydro-6- isoquinolinecarboxamide hydrochloride | 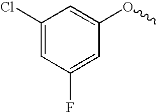 | 229 1,1-dimethylethyl 6-({[5- {[(3-chloro-5- fluorophenyl)oxy]methyl}- 1,3,4-thiadiazol-2- yl)amino]carbonyl}-3,4- dihydro-2(1H)- isoquinolinecarboxylate | HRMS (M + H)+: calculated for C₂₀H₁₆ClFN₄O₂S Theo: 419.0782 Found: 419.0745 Rt: 2.47 min MP: 308.2° C. |
| 63 N-[5-({[5-fluoro-2- (trifluoromethyl)phenyl]oxy} methyl)-1,3,4-thiadiazol- 2-yl]-1,2,3,4-tetrahydro-6- isoquinolinecarboxamide hydrochloride | 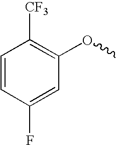 | 230 1,1-dimethylethyl 6-({[5- ({[5-fluoro-2- (trifluoromethyl)phenyl] oxy}methyl)-1,3,4- thiadiazol-2- yl]amino}carbonyl)-3,4- dihydro-2(1H)- isoquinolinecarboxylate | HRMS (M + H)+: calculated for C₂₀H₁₆F₄N₄O₂S Theo: 453.1008 Found: 453.0993 Rt: 2.48 min MP > 260° C. |
| 64 N-(5-{[(2,4- difluorophenyl)oxy]methyl}- 1,3,4-thiadiazol-2-yl)- 1,2,3,4-tetrahydro-6- isoquinolinecarboxamide hydrochloride | 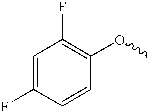 | 213 1,1-dimethylethyl 6-{[(5- {[(2,4- difluorophenyl)oxy]meth- yl}-1,3,4-thiadiazol-2- yl)amino]carbonyl}3,4- dihydro-2(1H)- isoquinolinecarboxylate | HRMS (M + H)+: calculated for C₁₉H₁₆F₂N₄O₂S Theo: 403.1040 Found: 403.1007 Rt: 2.25 min MP: 288.3° C. |

The following Examples were prepared using the generic reaction scheme (Scheme 14)

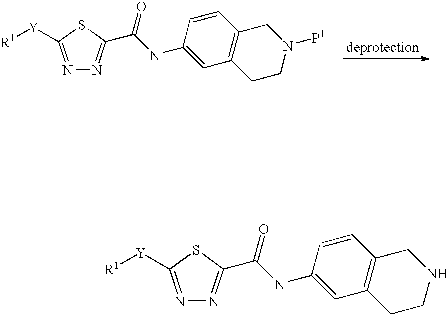

### EXAMPLE 65

### 5-{[(2-Chlorophenyl)oxy]methyl}-N-(1,2,3,4-tetrahydro-6-isoquinolinyl)-1,3,4-thiadiazole-2-carboxamide hydrochloride

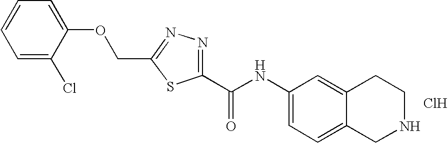

HCl(g) was bubbled at 0° C. in EtOAc until the solvent was saturated and 1,1-dimethylethyl 6-{[(5-{[(2-chlorophenyl)oxy]methyl}-1,3,4-thiadiazol-2-yl)carbonyl]amino}-3,4-dihydro-2(1H)-isoquinolinecarboxylate (Intermediate 236) (140 mg, 0.28 mmol) was added. The reaction mixture was stirred at room temperature for 3.5 hours. The resulting precipitate was filtered, washed with EtOAc and dried to give the title compound as an off white solid (115 mg, 94%).

HRMS calculated for C₁₉H₁₇ClN₄O₂S (M+H)⁺ 401.0839, found: 401.0853, Rt: 2.48 min.

MP: 285° C.

### EXAMPLE 66

### N-(5-{[(2-Chlorophenyl)(methyl)amino]methyl}-1,3,4-thiadiazol-2-yl)-1,2,3,4-tetrahydro-6-isoquinolinecarboxamide hydrochloride

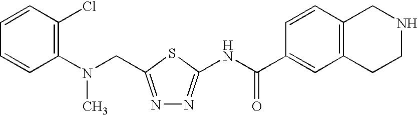

HCl(g) was bubbled at 0° C. in EtOAc until the solvent was saturated and 1,1-dimethylethyl 6-{[(5-{[(2-chlorophenyl)(methyl)amino]methyl}-1,3,4-thiadiazol-2-yl)amino]carbonyl}-3,4-dihydro-2(1H)-isoquinolinecarboxylate (Intermediate 241) (270 mg, 0.52 mmol) was added. The reaction mixture was stirred at room temperature overnight. The resulting precipitate was filtered, washed with EtOAc and dried to give the title compound as an off white solid (214 mg, 91%).

HRMS calculated for C₂₀H₂₀ClN₅OS (M+H)⁺ 414.1155, found: 414.1133, Rt: 2.10 min.

MP: 169-171° C.

### EXAMPLE 67

### N-(5-{[(2-Chlorophenyl)oxy]methyl}-1,3,4-thiadiazol-2-yl)-N-methyl-1,2,3,4-tetrahydro-6-isoquinolinecarboxamide hydrochloride

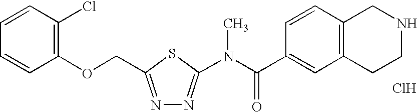

HCl(g) was bubbled at 0° C. in EtOAc until the solvent was saturated and 1,1-dimethylethyl 6-{[(5-{[(2-chlorophenyl)oxy]methyl}-1,3,4-thiadiazol-2-yl)(methyl)amino]carbonyl}-3,4-dihydro-2(1H)-isoquinolinecarboxylate (Intermediate 242) (138 mg, 0.27 mmol) was added. The reaction mixture was stirred at room temperature overnight. The resulting precipitate was filtered, washed with EtOAc and hot methyl alcohol and dried to give the title compound as a white solid (81 mg, 72%).

HRMS calculated for C₂₀H₁₉ClN₄O₂S (M+H)⁺415.0995, found: 415.0979, Rt: 2.59 min. MP>260° C.

The following Examples were prepared using the generic reaction scheme (Scheme 5)

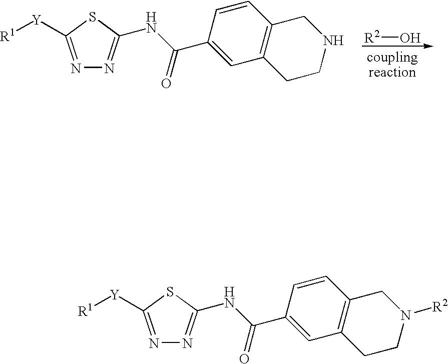

### EXAMPLE 68

### N-(5-{[(2-Chlorophenyl)oxy]methyl}-1,3,4-thiadiazol-2-yl)-2-(hydroxyacetyl)-1,2,3,4-tetrahydro-6-isoquinolinecarboxamide

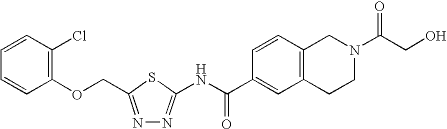

A solution of N-(5-{[(2-chlorophenyl)oxy]methyl}-1,3,4-thiadiazol-2-yl)-1,2,3,4-tetrahydro-6-isoquinolinecarboxamide (Example 1) (218 mg, 0.5 mmol), hydroxyacetic acid (46 mg, 0.6 mmol), HATU (247 mg, 0.65 mmol), triethylamine (132 mg, 1.3 mmol) in DMF was stirred at room temperature for 4 days. The DMF was evaporated under reduced pressure and the residue was dissolved in dichloromethane. The organic phase was then washed with a solution of sodium hydrogenocarbonate then dried over sodium sulphate. After filtration and evaporation under reduced pressure, the residue was purified by flash column chromatography eluting with a gradient DCM 100% to DCM/MeOH:98/2 to give the title compound as white solid (115 mg, 50%).

HRMS calculated for C₂₁H₁₉ClN₄O₄S (M+H)⁺ 459.0894, found: 459.0937, Rt: 2.56 min

MP: 133-135° C.

The following compounds were similarly prepared by a method analogous to that described for Example 68.

TABLE 21

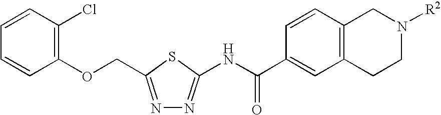

| Example No. | R² | From Example No. | Physical data |
|---|---|---|---|
| 69 N-(5-{[(2- chlorophenyl)oxy]methyl}- 1,3,4-thiadiazol-2-yl)-2- (2-hydroxy-2- methylpropanoyl)-1,2,3,4- tetrahydro-6- isoquinolinecarboxamide | 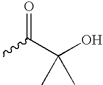 | 1 N-(5-{[(2- chlorophenyl)oxy]methyl}- 1,3,4-thiadiazol-2-yl)- 1,2,3,4-tetrahydro-6- isoquinolinecarboxamide | HRMS (M + H)+: calculated for C₂₃H₂₃ClN₄O₄S Theo: 487.1207 Found: 487.1206 Rt: 2.69 min MP: 196-198° C. |
| 70 N-(5-{[(2- chlorophenyl)oxy]methyl}- 1,3,4-thiadiazol-2-yl)-2- (N,N-dimethylglycyl)- 1,2,3,4-tetrahydro-6- isoquinolinecarboxamide | 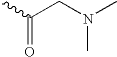 | 1 N-(5-{[(2- chlorophenyl)oxy]methyl}- 1,3,4-thiadiazol-2-yl)- 1,2,3,4-tetrahydro-6- isoquinolinecarboxamide | HRMS (M + H)+: calculated for C₂₃H₂₄ClN₅O₃S Theo: 486.1367 Found: 486.1364 Rt: 2.44 min MP: 109-111° C. |

The following Examples were prepared using the generic reaction scheme (Scheme 4)

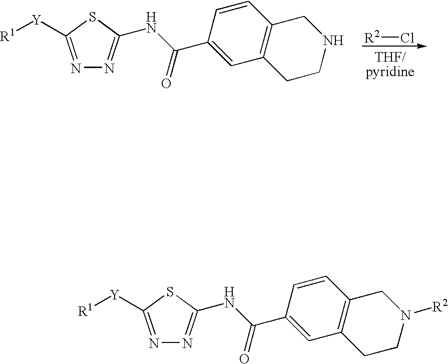

### EXAMPLE 71

### N-(5-{[(2-Chlorophenyl)oxy]methyl}-1,3,4-thiadiazol-2-yl)-2-(phenylcarbonyl)-1,2,3,4-tetrahydro-6-isoquinolinecarboxamide

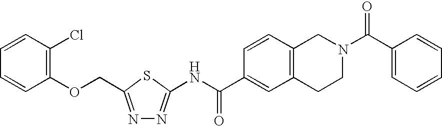

To a solution of N-(5-{[(2-chlorophenyl)oxy]methyl}-1,3,4-thiadiazol-2-yl)-1,2,3,4-tetrahydro-6-isoquinolinecarboxamide (Example 1) (210 mg, 0.48 mmol) in THF (10 mL) were added pyridine (0.232 mL, 2.88 mmol) then benzoyl chloride (0.122 mL, 1.06 mmol). The reaction mixture was stirred at room temperature overnight. The THF was evaporated under reduced pressure and the residue was dissolved in EtOAc. The organic phase was washed with water then dried over sodium sulphate. After filtration and evaporation under reduced pressure, the residue was purified by flash column chromatography eluting with a gradient DCM 100% to DCM/MeOH:98/2 to give the title compound as white solid (40 mg, 17%).

HRMS calculated for C₂₆H₂₁ClN₄O₃S (M+H)⁺505.1101, found: 505.1140, Rt: 2.99 min

MP: 186-188° C.

The following compounds were similarly prepared by a method analogous to that described for Example 71.

TABLE 22

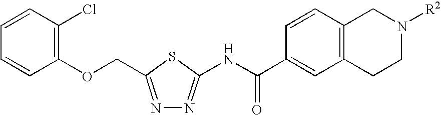

| Example No. | R² | From Example No. | Physical data |
|---|---|---|---|
| 72 2-butanoyl-N-(5-{[(2- chlorophenyl)oxy]meth- yl}-1,3,4-thiadiazol-2- yl)-1,2,3,4-tetrahydro- 6-isoquinoline carboxamide | 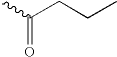 | 1 N-(5-{[(2- chlorophenyl)oxy]methyl}- 1,3,4-thiadiazol-2-yl)- 1,2,3,4-tetrahydro-6- isoquinolinecarboxamide | HRMS (M + H)+: calculated for C₂₃H₂₃ClN₄O₃S Theo: 471.1258 Found: 471.1242 Rt: 2.86 min MP: 187-189° C. |

The following Examples were prepared using the generic reaction scheme (Scheme 3)

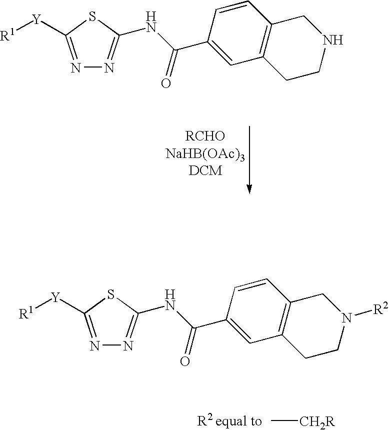

### EXAMPLE 73

### N-(5-{[(2-Chlorophenyl)oxy]methyl}-1,3,4-thiadiazol-2-yl)-2-propyl-1,2,3,4-tetrahydro-6-isoquinolinecarboxamide

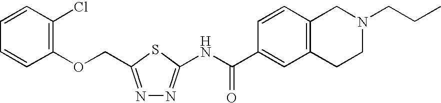

To a suspension of N-(5-{[(2-chlorophenyl)oxy]methyl}-1,3,4-thiadiazol-2-yl)-1,2,3,4-tetrahydro-6-isoquinolinecarboxamide (Example 1) (200 mg, 0.45 mmol) in DCM (10 mL) was added triethylamine (138 mg, 1.37 mmol). After stirring for 5 minutes, propanal (132 mg, 2.28 mmol) NaHB(OAc)₃ (483 mg, 2.28 mmol) and acetic acid (82 mg, 1.37 mmol) were added. The reaction mixture was stirred at room temperature for 2 days. The mixture was then washed with a solution of sodium hydrogenocarbonate and dried over sodium sulphate. After filtration and evaporation under reduced pressure, the residue was purified by flash column chromatography eluting with DCM to give the title compound as white solid (115 mg, 58%).

HRMS calculated for C₂₂H₂₃ClN₄O₂S (M+H)⁺ 443.1308, found: 443.1279, Rt: 2.97 min

MP: 186-188° C.

The following Examples were prepared using the generic reaction scheme (Scheme 7)

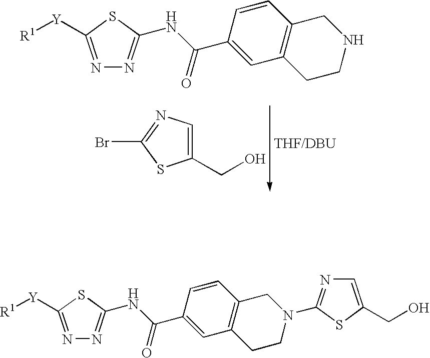

### EXAMPLE 74

### N-(5-{[(2-Chlorophenyl)oxy]methyl}-1,3,4-thiadiazol-2-yl)-2-[5-(hydroxymethyl)-1,3-thiazol-2-yl]-1,2,3,4-tetrahydro-6-isoquinolinecarboxamide

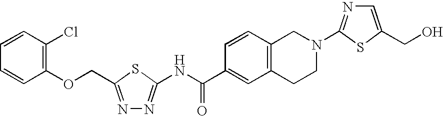

A mixture of N-(5-{[(2-chlorophenyl)oxy]methyl}-1,3,4-thiadiazol-2-yl)-1,2,3,4-tetrahydro-6-isoquinolinecarboxamide (Example 1) (218 mg, 0.5 mmol), DBU (0.224 mL, 1.5 mmol) and (2-bromo-1,3-thiazol-5-yl)methanol (97 mg, 0.5 mmol) in THF (10 mL) was stirred overnight at 80° C. Then extra amounts of DBU (0.075 mL, 0.5 mmol) and (2-bromo-1,3-thiazol-5-yl)methanol (97 mg, 0.5 mmol) were added and the mixture was stirred overnight at 60° C. The THF was evaporated under reduced pressure and the residue was dissolved in DCM. The organic phase was washed with water then dried over sodium sulphate. After filtration and evaporation under reduced pressure, the residue was purified by flash column chromatography eluting with a gradient DCM/MeOH:99/1 to DCM/MeOH:95/5 to give after trituration in cold DCM and drying the title compound as white solid (15 mg, 6%).

HRMS calculated for C₂₃H₂₀ClN₅O₃S₂ (M+H)⁺ 514.0775, found: 514.0770, Rt: 2.70 min

MP: 237-239° C.

The following Example was prepared using the generic reaction scheme (scheme 14)

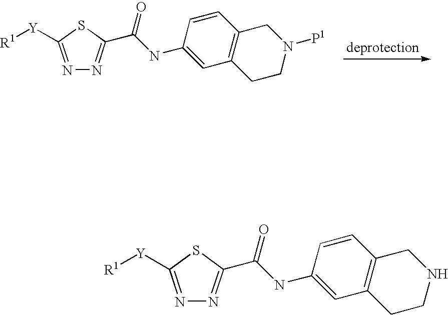

The following compounds were similarly prepared by analogous method to that described for Example 65.

TABLE 23

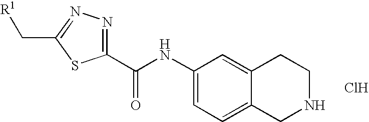

| Example No. | R¹ | From Intermediate No: | Physical data |
|---|---|---|---|
| 75 5-[(3,4- dichlorophenyl)methyl]-N- (1,2,3,4-tetrahydro-6- isoquinolinyl)-1,3,4- thiadiazole-2-carboxamide hydrochloride | 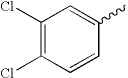 | 243 1,1-Dimethylethyl 6- [({5-[(3,4- dichlorophenyl)methyl]- 1,3,4-thiadiazol-2- yl}carbonyl)amino]-3,4- dihydro-2(1H)- isoquinolinecarboxylate | HRMS (M + H)+: calculated for C₁₉H₁₆Cl₂N₄OS Theo: 419.0500 Found: 419.0492 Rt: 2.59 min MP: 282.4° C. |

### Biological Assay

The compounds of the present invention may be analysed in vitro for SCD activity using an assay based on the production of [³H]H₂O, which is released during the enzyme-catalyzed generation of the monounsaturated fatty acyl CoA product. The assay is performed in a 96-well filtration plates. The titrated substrate used in the assay is the [9,10-³H] stearoyl Coenzyme A. After incubation for 6 minutes of SCD-containing rat microsomes (2 μg protein) and substrate (1 μM), the labelled fatty acid acyl-CoA species and microsomes are absorbed with charcoal and separated from [³H]H₂O by centrifugation. The formation of [³H]H₂O is used as a measure of SCD activity. Compounds at concentrations starting at 10 μM to 0.1 nM or vehicle (DMSO) are preincubated for 5 minutes with the microsomes before addition of the substrate. The concentration-responses are fitted with sigmoidal curves to obtain 1050 values.

All of the synthetic Example compounds 1-74 tested by the above described in vitro assay for SCD activity were found to exhibit an average pIC₅₀ value of greater than 5.5.

The following compounds were prepared according similar protocols to above described and when tested by the above described in vitro assay for SCD activity were found to exhibit an average pIC₅₀ value in the range 5-5.5.

| Structure | Name |
|---|---|
| 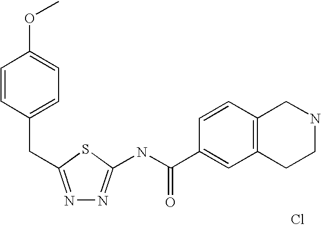 | N-(5-{[4-(methyloxy)phenyl]methyl}- 1,3,4-thiadiazol-2-yl)-1,2,3,4- tetrahydro-6- isoquinolinecarboxamide hydrochloride |
| 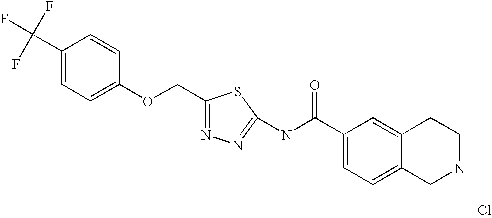 | N-[5-({[4- (trifluoromethyl)phenyl]oxy}methyl)- 1,3,4-thiadiazol-2-yl]-1,2,3,4- tetrahydro-6- isoquinolinecarboxamide hydrochloride |
| 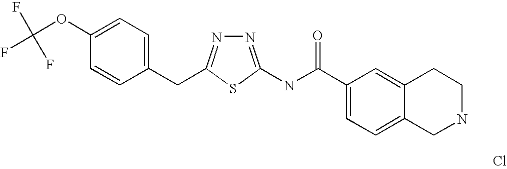 | N-[5-({4- [(trifluoromethyl)oxy]phenyl}methyl)- 1,3,4-thiadiazol-2-yl]-1,2,3,4- tetrahydro-6- isoquinolinecarboxamide hydrochloride |
| 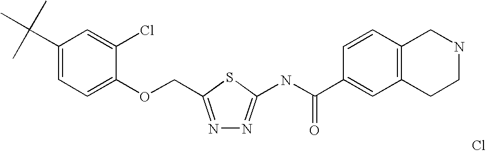 | N-[5-({[2-chloro-4-(1,1- dimethylethyl)phenyl]oxy}methyl)- 1,3,4-thiadiazol-2-yl]-1,2,3,4- tetrahydro-6- isoquinolinecarboxamide hydrochloride |

The following compounds were also prepared and when tested by the above described in vitro assay for SCD activity were found to exhibit an average pIC₅₀ value of less than 5.

| Structure | Name |
|---|---|
| 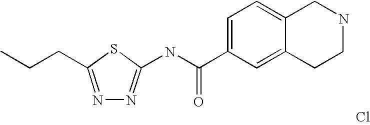 | N-(5-propyl-1,3,4-thiadiazol-2-yl)- 1,2,3,4-tetrahydro-6- isoquinolinecarboxamide hydrochloride |
| 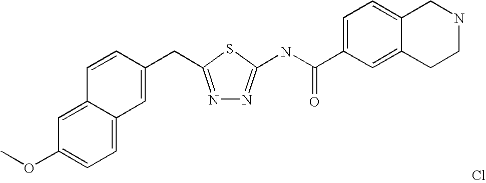 | N-(5-{[6-(methyloxy)-2- naphthalenyl]methyl}-1,3,4-thiadiazol- 2-yl)-1,2,3,4-tetrahydro-6- isoquinolinecarboxamide hydrochloride |
| 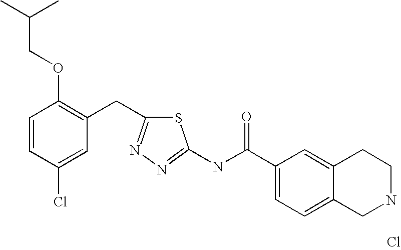 | N-[5-({5-chloro-2-[(2- methylpropyl)oxy]phenyl}methyl)-1,3,4- thiadiazol-2-yl]-1,2,3,4-tetrahydro-6- isoquinolinecarboxamide hydrochloride |
| 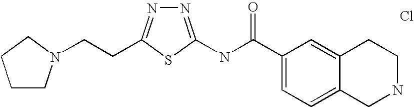 | N-{5-[2-(1-pyrrolidinyl)ethyl]-1,3,4- thiadiazol-2-yl}-1,2,3,4-tetrahydro-6- isoquinolinecarboxamide hydrochloride |
| 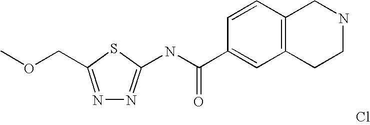 | N-{5-[(methyloxy)methyl]-1,3,4- thiadiazol-2-yl}-1,2,3,4-tetrahydro-6- isoquinolinecarboxamide hydrochloride |
| 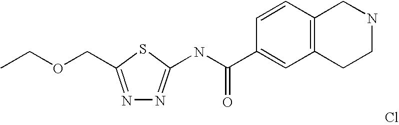 | N-{5-[(ethyloxy)methyl]-1,3,4- thiadiazol-2-yl}-1,2,3,4-tetrahydro-6- isoquinolinecarboxamide hydrochloride |
| 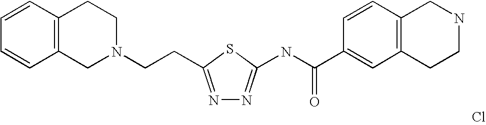 | N-{5-[2-(3,4-dihydro-2(1H)- isoquinolinyl)ethyl]-1,3,4-thiadiazol-2- yl}-1,2,3,4-tetrahydro-6- isoquinolinecarboxamide hydrochloride |
| 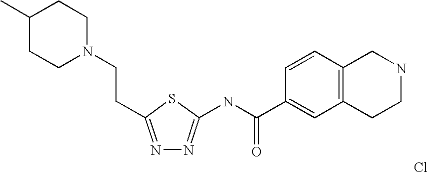 | N-{5-[2-(4-methyl-1-piperidinyl)ethyl]- 1,3,4-thiadiazol-2-yl}-1,2,3,4- tetrahydro-6-isoquinolinecarboxamide hydrochloride |
| 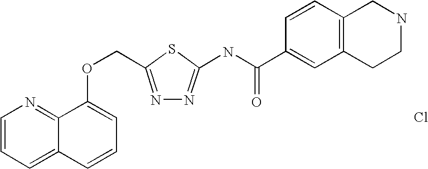 | N-{5-[(8-quinolinyloxy)methyl]-1,3,4- thiadiazol-2-yl}-1,2,3,4-tetrahydro-6- isoquinolinecarboxamide hydrochloride |
| 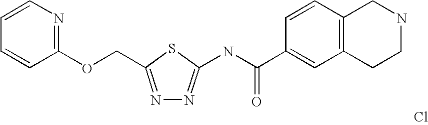 | N-{5-[(2-pyridinyloxy)methyl]-1,3,4- thiadiazol-2-yl}-1,2,3,4-tetrahydro-6- isoquinolinecarboxamide hydrochloride |
| 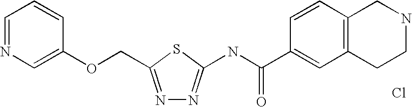 | N-{5-[(3-pyridinyloxy)methyl]-1,3,4- thiadiazol-2-yl}-1,2,3,4-tetrahydro-6- isoquinolinecarboxamide hydrochloride |
| 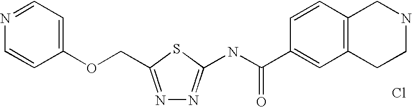 | N-{5-[(4-pyridinyloxy)methyl]-1,3,4- thiadiazol-2-yl}-1,2,3,4-tetrahydro-6- isoquinolinecarboxamide hydrochloride |
| 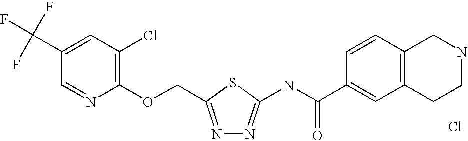 | N-[5-({[3-chloro-5-(trifluoromethyl)-2- pyridinyl]oxy}methyl)-1,3,4-thiadiazol- 2-yl]-1,2,3,4-tetrahydro-6- isoquinolinecarboxamide hydrochloride |
| 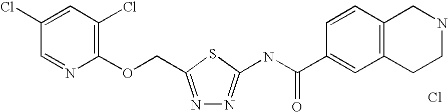 | N-(5-{[(3,5-dichloro-2- pyridinyl)oxy]methyl}-1,3,4-thiadiazol- 2-yl)-1,2,3,4-tetrahydro-6- isoquinolinecarboxamide hydrochloride |

1. A compound of formula (I): 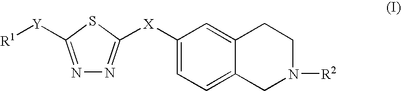
wherein:
X represents —CONH—, —NHCO— or —N(CH₃)CO—,
R¹ represents:
(i) H, —C₁₋₆alkyl or —C₃₋₆cycloalkyl, or
(ii) —C₆₋₁₀aryl optionally substituted by one, two or three groups independently selected from:
—C₁₋₆alkyl, —C₁₋₆haloalkyl, —C₃₋₆cycloalkyl, —C₁₋₆alkoxy, —CN, halogen,
—C₆₋₁₀aryl, —C₅₋₁₀heteroaryl and —C₅₋₁₀heterocyclyl, wherein the —C₆₋₁₀aryl, —C₅₋₁₀heteroaryl or —C₅₋₁₀heterocyclyl ring is optionally substituted by one, two or three groups independently selected from: —C₁₋₆alkyl, —C₁₋₆haloalkyl, —C₁₋₆alkoxy, —OR³, —CN, and halogen; or
(iii) —C₅₋₁₀heteroaryl or —C₅₋₁₀heterocyclyl wherein the —C₅₋₁₀heteroaryl or —C₅₋₁₀heterocyclyl is optionally substituted by one, two or three groups independently selected from:
—C₁₋₆alkyl, —C₁₋₆haloalkyl, —C₃₋₆cycloalkyl, —C₁₋₆alkoxy, —OR³, —CN, halogen,
—C₆₋₁₀aryl, —C₅₋₁₀heteroaryl and —C₅₋₁₀heterocyclyl wherein the —C₆₋₁₀aryl, —C₅₋₁₀heteroaryl or —C₅₋₁₀heterocyclyl ring is optionally substituted by one, two or three groups independently selected from: —C₁₋₆alkyl, —C₁₋₆haloalkyl, —C₁₋₆alkoxy, —OR³, —CN, and halogen,
Y represents —(CH₂)ₘ—, —O(CH₂)ₘ—, or —NR⁷(CH₂)ₘ—,
R² represents H, —C₁₋₆alkyl, —C(═O)C₁₋₆alkyl, —C(═O)C₃₋₆cycloalkyl —C(═O)C₆₋₁₀aryl, —C(═O)C₁₋₆alkylOH, —COC₁₋₃alkylNR⁴R⁵ or —C₅heteroarylR⁶,
R³ represents —C₁₋₆haloalkyl or —C₃₋₆cycloalkyl,
R⁴ represents H or —C₁₋₃alkyl,
R⁵ represents H or —C₁₋₃alkyl,
R⁶ represents —C₁₋₃alkylOH,
R⁷ represents H or —CH₃, and
m represents 1-4;
or a pharmaceutically acceptable salt thereof.

2. A compound of formula (I) or pharmaceutically acceptable salt thereof according to claim 1 wherein X represents —NHCO—.

3. A compound of formula (I) or pharmaceutically acceptable salt thereof according to claim 1 wherein R¹ represents —C₃₋₆cycloalkyl.

4. A compound of formula (I) or pharmaceutically acceptable salt thereof according to claim 1 wherein R¹ represents —C₆₋₁₀aryl optionally substituted by: one, two or three groups independently selected from:
—C₁₋₆alkyl, —C₁₋₆haloalkyl, —C₃₋₆cycloalkyl, —C₁₋₆alkoxy, —OR³, —CN, halogen, and
—C₆₋₁₀aryl optionally substituted by one, two or three groups independently selected from: —C₁₋₆alkyl, —C₁₋₆alkoxy, —OR³, —C₁₋₆haloalkyl, —CN and halogen.

5. A compound of formula (I) or pharmaceutically acceptable salt thereof according to claim 1 wherein Y represents —CH₂, —OCH₂—, —OCH₂CH₂—, —C₂H₄— or —N(CH₃)CH₂—.

6. A compound of formula (I) or pharmaceutically acceptable salt thereof according to claim 1 wherein R² represents hydrogen.

7. A compound of formula (I) according to claim 1 selected from:
N-(5-{[(2-chlorophenyl)oxy]methyl}-1,3,4-thiadiazol-2-yl)-1,2,3,4-tetrahydro-6-isoquinolinecarboxamide,
N-[5-(1-naphthalenylmethyl)-1,3,4-thiadiazol-2-yl]-1,2,3,4-tetrahydro-6-isoquinolinecarboxamide,
N-(5-{[(3,4-dichlorophenyl)oxy]methyl}-1,3,4-thiadiazol-2-yl)-1,2,3,4-tetrahydro-6-isoquinolinecarboxamide,
N-(5-{[(4-chlorophenyl)oxy]methyl}-1,3,4-thiadiazol-2-yl)-1,2,3,4-tetrahydro-6-isoquinolinecarboxamide,
N-[5-(phenylmethyl)-1,3,4-thiadiazol-2-yl]-1,2,3,4-tetrahydro-6-isoquinolinecarboxamide,
N-{5-[(4-chlorophenyl)methyl]-1,3,4-thiadiazol-2-yl}-1,2,3,4-tetrahydro-6-isoquinolinecarboxamide,
N-{5-[(3,4-dichlorophenyl)methyl]-1,3,4-thiadiazol-2-yl}-1,2,3,4-tetrahydro-6-isoquinolinecarboxamide,
N-[5-(2-thienylmethyl)-1,3,4-thiadiazol-2-yl]-1,2,3,4-tetrahydro-6-isoquinolinecarboxamide,
N-[5-(2-naphthalenylmethyl)-1,3,4-thiadiazol-2-yl]-1,2,3,4-tetrahydro-6-isoquinolinecarboxamide,
N-[5-(cyclohexylmethyl)-1,3,4-thiadiazol-2-yl]-1,2,3,4-tetrahydro-6-isoquinolinecarboxamide,
N-[5-(2-phenylethyl)-1,3,4-thiadiazol-2-yl]-1,2,3,4-tetrahydro-6-isoquinolinecarboxamide,
N-[5-(1H-indol-3-ylmethyl)-1,3,4-thiadiazol-2-yl]-1,2,3,4-tetrahydro-6-isoquinolinecarboxamide,
N-(5-{[(2,5-dichlorophenyl)oxy]methyl}-1,3,4-thiadiazol-2-yl)-1,2,3,4-tetrahydro-6-isoquinolinecarboxamide,
N-{5-[(1-naphthalenyloxy)methyl]-1,3,4-thiadiazol-2-yl}-1,2,3,4-tetrahydro-6-isoquinolinecarboxamide,
N-(5-{[(2-chloro-4-fluorophenyl)oxy]methyl}-1,3,4-thiadiazol-2-yl)-1,2,3,4-tetrahydro-6-isoquinolinecarboxamide,
N-(5-{[(2-chloro-5-fluorophenyl)oxy]methyl}-1,3,4-thiadiazol-2-yl)-1,2,3,4-tetrahydro-6-isoquinolinecarboxamide,
N-[5-(1-benzothien-3-ylmethyl)-1,3,4-thiadiazol-2-yl]-1,2,3,4-tetrahydro-6-isoquinolinecarboxamide,
N-[5-(3-thienylmethyl)-1,3,4-thiadiazol-2-yl]-1,2,3,4-tetrahydro-6-isoquinolinecarboxamide,
N-{5-[2-(1-naphthalenyl)ethyl]-1,3,4-thiadiazol-2-yl}-1,2,3,4-tetrahydro-6-isoquinolinecarboxamide,
N-{5-[2-(2-chlorophenyl)ethyl]-1,3,4-thiadiazol-2-yl}-1,2,3,4-tetrahydro-6-isoquinolinecarboxamide,
N-{5-[(2-bromophenyl)methyl]-1,3,4-thiadiazol-2-yl}-1,2,3,4-tetrahydro-6-isoquinolinecarboxamide,
N-(5-{[(2-fluorophenyl)oxy]methyl}-1,3,4-thiadiazol-2-yl)-1,2,3,4-tetrahydro-6-isoquinolinecarboxamide,
N-(5-{[(3-fluorophenyl)oxy]methyl}-1,3,4-thiadiazol-2-yl)-1,2,3,4-tetrahydro-6-isoquinolinecarboxamide,
N-(5-{[(4-fluorophenyl)oxy]methyl}-1,3,4-thiadiazol-2-yl)-1,2,3,4-tetrahydro-6-isoquinolinecarboxamide,
N-(5-{[(3-chlorophenyl)oxy]methyl}-1,3,4-thiadiazol-2-yl)-1,2,3,4-tetrahydro-6-isoquinolinecarboxamide,
N-[5-({[2-(trifluoromethyl)phenyl]oxy}methyl)-1,3,4-thiadiazol-2-yl]-1,2,3,4-tetrahydro-6-isoquinolinecarboxamide,
N-[5-({[3-(trifluoromethyl)phenyl]oxy}methyl)-1,3,4-thiadiazol-2-yl]-1,2,3,4-tetrahydro-6-isoquinolinecarboxamide,
N-(5-{[3-(trifluoromethyl)phenyl]methyl}-1,3,4-thiadiazol-2-yl)-1,2,3,4-tetrahydro-6-isoquinolinecarboxamide,
N-{5-[(5,6,7,8-tetrahydro-1-naphthalenyloxy)methyl]-1,3,4-thiadiazol-2-yl}-1,2,3,4-tetrahydro-6-isoquinolinecarboxamide,
N-{5-[(2-chlorophenyl)methyl]-1,3,4-thiadiazol-2-yl}-1,2,3,4-tetrahydro-6-isoquinolinecarboxamide,
N-(5-{[2-(trifluoromethyl)phenyl]methyl}-1,3,4-thiadiazol-2-yl)-1,2,3,4-tetrahydro-6-isoquinolinecarboxamide,
N-[5-({[4-(methyloxy)phenyl]oxy}methyl)-1,3,4-thiadiazol-2-yl]-1,2,3,4-tetrahydro-6-isoquinolinecarboxamide,
N-{5-[(2-biphenylyloxy)methyl]-1,3,4-thiadiazol-2-yl}-1,2,3,4-tetrahydro-6-isoquinolinecarboxamide,
N-(5-{[4-(trifluoromethyl)phenyl]methyl}-1,3,4-thiadiazol-2-yl)-1,2,3,4-tetrahydro-6-isoquinolinecarboxamide,
N-{5-[({5-chloro-2-[(2-methylpropyl)oxy]phenyl}oxy)methyl]-1,3,4-thiadiazol-2-yl}-1,2,34-tetrahydro-6-isoquinolinecarboxamide,
N-{5-[(4-fluorophenyl)methyl]-1.3.4-thiadiazol-2-yl}-1,2,3,4-tetrahydro-6-isoquinolinecarboxamide,
N-[5-({[2-(methyloxy)phenyl]oxy}methyl)-1,3,4-thiadiazol-2-yl]-1,2,3,4-tetrahydro-6-isoquinolinecarboxamide,
N-{5-[(1-methyl-1H-indol-3-yl)methyl]-1,3,4-thiadiazol-2-yl}-1,2,3,4-tetrahydro-6-isoquinolinecarboxamide,
N-[5-(3-pyridinylmethyl)-1,3,4-thiadiazol-2-yl]-1,2,3,4-tetrahydro-6-isoquinolinecarboxamide,
N-[5-(5,6,7,8-tetrahydro-2-naphthalenylmethyl)-1,3,4-thiadiazol-2-yl]-1,2,3,4-tetrahydro-6-isoquinolinecarboxamide,
N-[5-(3,4-dihydro-2H-chromen-6-ylmethyl)-1,3,4-thiadiazol-2-yl]-1,2,3,4-tetrahydro-6-isoquinolinecarboxamide,
N-(5-{2-[(2-chlorophenyl)oxy]ethyl}-1,3,4-thiadiazol-2-yl)-1,2,3,4-tetrahydro-6-isoquinolinecarboxamide,
N-(5-{[(2,4-dichlorophenyl)oxy]methyl}-1,3,4-thiadiazol-2-yl)-1,2,3,4-tetrahydro-6-isoquinolinecarboxamide,
N-{5-[(2′-chloro-2-biphenylyl)methyl]-1,3,4-thiadiazol-2-yl}-1,2,3,4-tetrahydro-6-isoquinolinecarboxamide,
N-{5-[(2-fluorophenyl)methyl]-1,3,4-thiadiazol-2-yl}-1,2,3,4-tetrahydro-6-isoquinolinecarboxamide,
N-{5-[(3-chlorophenyl)methyl]-1,3,4-thiadiazol-2-yl}-1,2,3,4-tetrahydro-6-isoquinolinecarboxamide,
N-(5-{[(2,6-dichlorophenyl)oxy]methyl}-1,3,4-thiadiazol-2-yl)-1,2,3,4-tetrahydro-6-isoquinolinecarboxamide,
N-(5-{[(2-methylphenyl)oxy]methyl}-1.3.4-thiadiazol-2-yl)-1,2,3,4-tetrahydro-6-isoquinolinecarboxamide,
N-(5-{[(3,4-dimethylphenyl)oxy]methyl}-1,3,4-thiadiazol-2-yl)-1,2,3,4-tetrahydro-6-isoquinolinecarboxamide,
N-{5-[(2,4-dichlorophenyl)methyl]-1,3,4-thiadiazol-2-yl}-1,2,3,4-tetrahydro-6-isoquinolinecarboxamide,
N-[5-({2-[(trifluoromethyl)oxy]phenyl}methyl)-1,3,4-thiadiazol-2-yl]-1,2,3,4-tetrahydro-6-isoquinolinecarboxamide,
N-(5-{[(2-chloro-3,5-difluorophenyl)oxy]methyl}-1,3,4-thiadiazol-2-yl)-1,2,3,4-tetrahydro-6-isoquinolinecarboxamide,
N-(5-{[(2-chloro-6-fluorophenyl)oxy]methyl}-1,3,4-thiadiazol-2-yl)-1,2,3,4-tetrahydro-6-isoquinolinecarboxamide,
N-[5-({[2-chloro-3-(trifluoromethyl)phenyl]oxy}methyl)-1,3,4-thiadiazol-2-yl]-1,2,3,4-tetrahydro-6-isoquinolinecarboxamide,
N-(5-{[(2,4,5-trichlorophenyl)oxy]methyl}-1,3,4-thiadiazol-2-yl)-1,2,3,4-tetrahydro-6-isoquinolinecarboxamide,
N-[5-({[2-chloro-5-(trifluoromethyl)phenyl]oxy}methyl)-1,3,4-thiadiazol-2-yl]-1,2,3,4-tetrahydro-6-isoquinolinecarboxamide,
N-{5-[(4-chloro-2-fluorophenyl)methyl]-1,3,4-thiadiazol-2-yl}-1,2,3,4-tetrahydro-6-isoquinolinecarboxamide,
N-(5-{[4-fluoro-2-(trifluoromethyl)phenyl]methyl}-1,3,4-thiadiazol-2-yl)-1,2,3,4-tetrahydro-6-isoquinolinecarboxamide,
N-(5-{[5-chloro-2-(trifluoromethyl)phenyl]methyl}-1,3,4-thiadiazol-2-yl)-1,2,3,4-tetrahydro-6-isoquinolinecarboxamide,
N-[5-({[4-fluoro-2-(trifluoromethyl)phenyl]oxy}methyl)-1,3,4-thiadiazol-2-yl]-1,2,3,4-tetrahydro-6-isoquinolinecarboxamide,
N-[5-({[2-chloro-4-(trifluoromethyl)phenyl]oxy}methyl)-1,3,4-thiadiazol-2-yl]-1,2,3,4-tetrahydro-6-isoquinolinecarboxamide,
N-(5-{[(3-chloro-5-fluorophenyl)oxy]methyl}-1,3,4-thiadiazol-2-yl)-1,2,3,4-tetrahydro-6-isoquinolinecarboxamide,
N-[5-({[5-fluoro-2-(trifluoromethyl)phenyl]oxy}methyl)-1,3,4-thiadiazol-2-yl]-1,2,3,4-tetrahydro-6-isoquinolinecarboxamide,
N-(5-{[(2,4-difluorophenyl)oxy]methyl}-1,3,4-thiadiazol-2-yl)-1,2,3,4-tetrahydro-6-isoquinolinecarboxamide,
5-{[(2-chlorophenyl)oxy]methyl}-N-(1,2,3,4-tetrahydro-6-isoquinolinyl)-1,3,4-thiadiazole-2-carboxamide,
N-(5-{[(2-chlorophenyl)(methyl)amino]methyl}-1,3,4-thiadiazol-2-yl)-1,2,3,4-tetrahydro-6-isoquinolinecarboxamide,
N-(5-{[(2-chlorophenyl)oxy]methyl}-1,3,4-thiadiazol-2-yl)-N-methyl-1,2,3,4-tetrahydro-6-isoquinolinecarboxamide,
N-(5-{[(2-chlorophenyl)oxy]methyl}-1,3,4-thiadiazol-2-yl)-2-(hydroxyacetyl)-1,2,3,4-tetrahydro-6-isoquinolinecarboxamide,
N-(5-{[(2-chlorophenyl)oxy]methyl}-1,3,4-thiadiazol-2-yl)-2-(2-hydroxy-2-methylpropanoyl)-1,2,3,4-tetrahydro-6-isoquinolinecarboxamide,
N-(5-{[(2-chlorophenyl)oxy]methyl}-1,3,4-thiadiazol-2-yl)-2-(N,N-dimethylglycyl)-1,2,3,4-tetrahydro-6-isoquinolinecarboxamide,
N-(5-{[(2-chlorophenyl)oxy]methyl}-1,3,4-thiadiazol-2-yl)-2-(phenylcarbonyl)-1,2,3,4-tetrahydro-6-isoquinolinecarboxamide,
2-butanoyl-N-(5-{[(2-chlorophenyl)oxy]methyl}-1,3,4-thiadiazol-2-yl)-1,2,3,4-tetrahydro-6-isoquinolinecarboxamide,
N-(5-{[(2-chlorophenyl)oxy]methyl}-1,3,4-thiadiazol-2-yl)-2-propyl-1,2,3,4-tetrahydro-6-isoquinolinecarboxamide,
N-(5-{[(2-chlorophenyl)oxy]methyl}-1,3,4-thiadiazol-2-yl)-2-[5-(hydroxymethyl)-1,3-thiazol-2-yl]-1,2,3,4-tetrahydro-6-isoquinolinecarboxamide, and
5-[(3,4-dichlorophenyl)methyl]-N-(1,2,3,4-tetrahydro-6-isoquinolinyl)-1,3,4-thiadiazole-2-carboxamide,
or a pharmaceutically acceptable salt thereof.

8. A pharmaceutical composition comprising a compound of formula (I) or pharmaceutically acceptable salt thereof according to claim 1 together with at least one pharmaceutical carrier and/or excipient.

9. (canceled)

10. (canceled)

11. (canceled)

12. (canceled)

13. (canceled)

14. (canceled)

15. (canceled)

16. A method of treating and/or preventing a disease or a condition susceptible to amelioration by an SCD inhibitor comprising administering to a subject a therapeutically effective amount of a compound of formula (I) or pharmaceutically acceptable salt thereof according to claim 1.

17. A method of treating and/or preventing diseases or conditions caused by or associated with an abnormal plasma lipid profile selected from dyslipidemia, hypoalphalipoproteinemia, hyperbetalipoproteinemia, hypercholesterolemia, hypertriglyceridemia, familial hypercholesterolemia, angina, ischemia, cardiac ischemia, stroke, myocardial infarction, atherosclerosis, obesity, Type I diabetes, Type II diabetes, insulin resistance, hyperinsulinaemia and metabolic syndrome; cardiovascular diseases selected from peripheral vascular disease, reperfusion injury, angioplastic restenosis, hypertension, vascular complications of diabetes, and thrombosis; hepatic steatosis, non-alcoholic steatohepatitis (NASH) or other diseases related to accumulation of lipids in the liver; skin disorders selected from eczema, acne, psoriasis, and keloid scar formation; diseases related to production or secretions from mucous membranes; cancer, neoplasia, malignancy, metastases, tumours (benign or malignant), carcinogenesis, or hepatomas; or mild cognitive impairment (MCI), Alzheimer's Disease (AD), cerebral amyloid angiopathy (CM) or dementia associated with Down Syndrome (DS) or other neurodegenerative diseases characterized by the formation or accumulation of amyloid plaques comprising Aβ42; comprising administering to a subject a therapeutically effective amount of a compound of formula (I) or pharmaceutically acceptable salt thereof according to claim 1.

18. A method of treating and/or preventing acne, dyslipidemia, hypertriglyceridemia, atherosclerosis, obesity, Type II diabetes, insulin resistance, hyperinsulinaemia, hepatic steatosis and/or non-alcoholic steatohepatitis (NASH) comprising administering to a subject a therapeutically effective amount of a compound of formula (I) or pharmaceutically acceptable salt thereof according to claim 1.

19. A compound of formula (I) or a pharmaceutically acceptable salt thereof according to claim 1 in combination with one or more active agent(s) selected from an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA, a cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor; wherein the compound according to claim 1 and the one or more active agent(s) are in the same or separate formulations.

20. The method of claim 16 wherein the compound of formula (I) or a pharmaceutically acceptable salt thereof according to claim 1 is administered in combination with one or more active agent(s) selected from an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA, a cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor.
